(19) United States
(12) Patent Application Publication
(10) Pub. No.: US 2026/0055404 A1
(43) Pub. Date: Feb. 26, 2026

(54) EXTRACELLULAR VESICLE COMPRISING A BIOLOGICALLY ACTIVE MOLECULE AND A CELL PENETRATING PEPTIDE CLEAVABLE LINKER

(71) Applicant: LONZA SALES AG, Basel (CH)

(72) Inventors: Aaron NOYES, Basel (CH); Yaozhong ZHANG, Basel (CH)

(21) Appl. No.: 19/103,902

(22) PCT Filed: Aug. 15, 2023
(86) PCT No.: PCT/US2023/072243

§ 371 Date: Feb. 14, 2025

Related U.S. Application Data
(60) Provisional application No. 63/371,711, filed on Aug. 17, 2022.

(51) Int. Cl. C12N 15/113; A61K 47/54; A61K 47/65; A61K 47/69

(52) U.S. Cl. CPC C12N 15/113; A61K 47/544; A61K 47/554; A61K 47/65; A61K 47/6901

(57) ABSTRACT

The present disclosure relates to extracellular vesicles (e.g., exosomes) comprising a biologically active molecule covalently linked to the extracellular vesicle via a cleavable linker comprising a cell penetrating peptide and an anchoring moiety. The extracellular vesicles can be useful as an agent for the prophylaxis or treatment of cancer or other diseases. Also provided herein are methods for producing the extracellular vesicles and methods for using the extracellular vesicles to treat diseases or disorders.

### REFERENCE TO SEQUENCE LISTING SUBMITTED ELECTRONICALLY VIA EFS-WEB

The content of the electronically submitted sequence listing (Name: 0132-0311WO1_Seqlisting_ST26.xml, Size: 1,523,889 bytes; and Date of Creation: Aug. 16, 2022) submitted in this application is incorporated herein by reference in its entirety.

### TECHNICAL FIELD

The present disclosure provides extracellular vesicles (EVs), e.g., exosomes, which can be useful as an agent for the prophylaxis or treatment of cancer and other diseases comprising at least one biologically active molecule linked to the extracellular vesicle, e.g., exosome, via a cleavable linker and an anchoring moiety.

### BACKGROUND

Many bioactive compounds have potent biological activity that is of therapeutic interest. However, these compounds often exhibit toxicity in non-target organs. One way to limit exposure of non-target tissues is to chemically conjugate small molecules to affinity-based reagents such as antibodies, which can direct the therapeutic compound to specific cell types (Dosio, F. et al., Toxins (Basel) 3 (7): 848-883 (2011)), but this approach is limited by the number of molecules of the compound of interest that can be attached to an antibody (typically 2-6 molecules per antibody), and by the availability/existence of antibodies that specifically bind to targeted, relevant diseased/effector cells without binding to non-target cells. These two issues limit the use of antibody-drug conjugates (ADC) by decreasing potency and increasing systemic toxicity, respectively. Accordingly, there is a need for delivery systems with a higher payload than ADCs that can selectively target specific tissues or organs while at the same time limiting overall systemic exposure to the therapeutic compound.

EVs, e.g., exosomes, are important mediators of intercellular communication. They are also important biomarkers in the diagnosis and prognosis of many diseases, such as cancer. As drug delivery vehicles, EVs, e.g., exosomes, offer many advantages over traditional drug delivery methods (e.g., peptide immunization, DNA vaccines) as a new treatment modality in many therapeutic areas. However, despite its advantages, many EVs, e.g., exosomes, have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5 (4): e1071008 (2015).

Accordingly, new and more effective engineered-EVs, e.g., exosomes, are necessary to better enable therapeutic use and other applications of EV-based technologies.

### BRIEF SUMMARY

The present disclosure provides an extracellular vesicle (EV) comprising a biologically active molecule (BAM) attached to the EV via an anchoring moiety (AM) according to Formula I or II:

$$AM - {SP}_{1} - {L}_{1} - {SP}_{2} - {L}_{2} - {SP}_{3} - BAM - {SP}_{4} - {L}_{3} \qquad ( Formula I )$$

$$AM - {SP}_{1} - {L}_{1} - {SP}_{2} - {L}_{2} - {SP}_{3} - BAM \qquad ( Formula II )$$

wherein
L₁, L₂, and L₃ are the same or different and each is an optional cleavable linkage; and
SP₁, SP₂, SP₃, and SP₄ are optional first, second, third, and fourth spacers, respectively, and
   wherein at least one of L₁, L₂, and L₃ is present and comprises a cell penetrating peptide.

In some aspects, AM is covalently linked to BAM at the 5′ position. In some aspects, AM is covalently linked to BAM at the 3′ position.

In some aspects, the cell penetrating peptide comprises three or more argininyl moieties. In some aspects, the cell penetrating peptide comprises three, six, or nine argininyl moieties. In some aspects, the cell penetrating peptide further comprises at least one amino acid other than argininyl, such as cysteinyl, glycinyl, or a combination thereof. In some aspects, the cell penetrating peptide comprises a cyclic peptide, TAT, or Antp (antennapedia).

In some aspects, L₁ is present in Formula I or II and comprises the cell penetrating peptide. In some aspects, L₂ is present in Formula I or II and comprises the cell penetrating peptide. In some aspects, L₃ is present in Formula I and comprises the cell penetrating peptide.

In some aspects, at least one cleavable linkage of L₁, L₂, and L₃ that does not comprise the cell penetrating peptide is present and is a cleavable linkage comprising a phosphodiester bond, a disulfido, a polypeptidyl, a polynucleotidyl, a pyrophosphato, or a silyl ether or a combination thereof.

In some aspects, the at least one cleavable linkage that does not comprise the cell penetrating peptide is a cleavable linkage comprising a phosphodiester.

In some aspects, the at least one cleavable linkage that does not comprise the cell penetrating peptide is a cleavable linkage comprising a disulfido.

In some aspects, the at least one cleavable linkage that does not comprise the cell penetrating peptide is a cleavable linkage comprising a polypeptidyl. In some aspects, the polypeptidyl is selected from alanine-alanine-asparagine, valine-glycine, glycine-glycine, glutamic acid-valine-citrulline, aspartic acid-valine-citrulline, serine-valine-citrulline, lysine-valine-citrulline, glycine-glycine-glycine-valine-citrulline, cyclobutane-1,1-dicarboxamide-citrulline, and alanine-phenylalanine-lysine.

In some aspects, the at least one cleavable linkage that does not comprise the cell penetrating peptide is a cleavable linkage comprising a polynucleotidyl. In some aspects, the polynucleotidyl is a trinucleotidyl or higher. In some aspects, the polynucleotidyl is a tetranucleotidyl comprising dTdTdTdT, wherein dT is deoxythymidine.

In some aspects, the at least one cleavable linkage that does not comprise the cell penetrating peptide is a cleavable linkage comprising a pyrophosphato.

In some aspects, the at least one cleavable linkage that does not comprise the cell penetrating peptide is a cleavable linkage comprising a silyl ether. In some aspects, the silyl ether comprises-OSiR R²O—, wherein R¹ and R² are the same or different and each is C1-8 alkyl or aryl. In some aspects, the silyl ether comprises-OSiR ′R²O—, wherein R¹ and R² are both isopropyl.

In some aspects, the AM comprises a sterol, a lipid, a vitamin, a peptide, or a combination thereof.

In some aspects, the AM comprises a sterol comprising cholesterol, thiocholesterol, ergosterol, 7-dehydrocholesterol, 24S-hydroxycholesterol, lanosterol, cycloartenol, fucosterol, saringosterol, campesterol, β-sitosterol, sitostanol, coprostanol, avenasterol, stigmasterol, or combinations thereof. In some aspects, the sterol is cholesterol.

In some aspects, the AM comprises a lipid comprising a fatty acid or a phospholipid. In some aspects, the fatty acid is a straight chain fatty acid, a branched fatty acid, a saturated fatty acid, an unsaturated fatty acid, a hydroxyl fatty acid, a polycarboxylic acid, or any combination thereof. In some aspects, the straight chain fatty acid is butyric acid, caproic acid, caprylic acid, capric acid, lauric acid, myristic acid, palmitic acid, or stearic acid. In some aspects, the straight chain fatty acid is palmitic acid. In some aspects, the phospholipid comprises 16:0 1,2-dipalmitoyl-sn-glycero-3-phosphoethanolamine-N-[3-(2-pyridyldithio) propionate] (16:0 PDP PE), 16:0 1,2-dipalmitoyl-sn-glycero-3-phosphoethanolamine-N-[4-(p-maleimidomethyl)cyclohexane-carboxamide] (16:0 PE MCC), or 16:0 1,2-dipalmitoyl-sn-glycero-3-phosphoethanolamine-N-(cyanur)(16:0 Cyanur PE).

In some aspects, the AM comprises a vitamin comprising tocopherol, tocotrienol, vitamin D, vitamin K, riboflavin, niacin, or pyridoxine. In some aspects, the vitamin is tocopherol. In some aspects, the AM is attached to an exterior surface of the EV.

In some aspects, the BAM comprises a peptide, a polypeptidyl, a polynucleotidyl, a protein, an antibody or an antigen binding fragment thereof, a chemical compound, or any combination thereof. In some aspects, BAM comprises an antisense oligonucleotidyl (ASO), siRNA, miRNA, shRNA, a nucleic acid, or any combination thereof. In some aspects, BAM comprises an ASO. In some aspects, the ASO targets a transcript. In some aspects, the transcript is a STAT6 transcript, an EGFP transcript, a CEBP/β transcript, a STAT3 transcript, a KRAS transcript, an NRAS transcript, an NLPR3 transcript, an FFLUC transcript, an RLUC transcript, a MYC transcript, or any combination thereof.

In some aspects, SP₁, SP₂, SP₃, and SP₄ are the same or different and each comprises an alkylenyl, a polyoxyalkylenyl, a succinimido, a maleimido, an aryl, an ether, a carbonyl, a carboxylato, a carbamoyl, a thioether, a sulfo, a thiocarbonyl, a thiocarbamoyl, a thiosuccinimido, an amino, an amido, a hydrazido, a phosphorothioato, a 1,2,3-triazolyl, a dibenzoylcyclooctenyl, a bicyclononenyl, a p-aminobenzoyl, a p-aminobenzylcarbamato, or a combination thereof, and at least one of SP₁, SP₂, SP₃, and SP₄ is present. In some aspects, at least one of SP₁, SP₂, SP₃, and SP₄ comprises C₁₋₈ alkylenyl, polyoxyalkenyl, a maleimido, a carbamoyl, a thio, an amido, a 1,2,3-triazolyl, a dibenzoylcyclooctenyl, a bicyclononenyl, a p-aminobenzoyl, a p-aminobenzylcarbamato, or a combination thereof. In some aspects, at least one of SP₁, SP₂, SP₃, and SP₄ comprises C₁₋₆ alkylenyl (i.e., C₁ alkylenyl, C₂ alkylenyl, C₃ alkylenyl, C₄ alkylenyl, C₅ alkylenyl, or C₆ alkylenyl). In some aspects, at least one of SP₁, SP₂, SP₃, and SP₄ comprises a polyoxyalkylenyl that comprises 2 to 15-OCH₂CH₂— repeat units. In some aspects, wherein at least one of SP₁, SP₂, SP₃, and SP₄ further comprises a carbamoyl, an amino, an amido, a thiosuccinimido, a 1,2,3-triazolylbicyclononenyl, or a combination thereof.

In some aspects, SP₁ is present in Formula I or II. In some aspects, SP₂ is present in Formula I or II. In some aspects, SP₃ is present in Formula I or II. In some aspects, SP₄ is present in Formula I. In some aspects, SP₁ and SP₂ are both present in Formula I or II. In some aspects, SP₁ and SP₃ are both present in Formula I or II. In some aspects, SP₁ and SP₄ are both present in Formula I. In some aspects, SP₂ and SP₃ are both present in Formula I or II. In some aspects, SP₂ and SP₄ are both present in Formula I. In some aspects, SP₃ and SP₄ are both present in Formula I. In some aspects, SP₁, SP₂, and SP₃ are present in Formula I or II. In some aspects, SP₁, SP₂, and SP₄ are present in Formula I. In some aspects, SP₂, SP₃, and SP₄ are present in Formula I. In some aspects, SP₁, SP₂, SP₃, and SP₄ are present in Formula I.

In some aspects, Formula I or II is a construct selected from

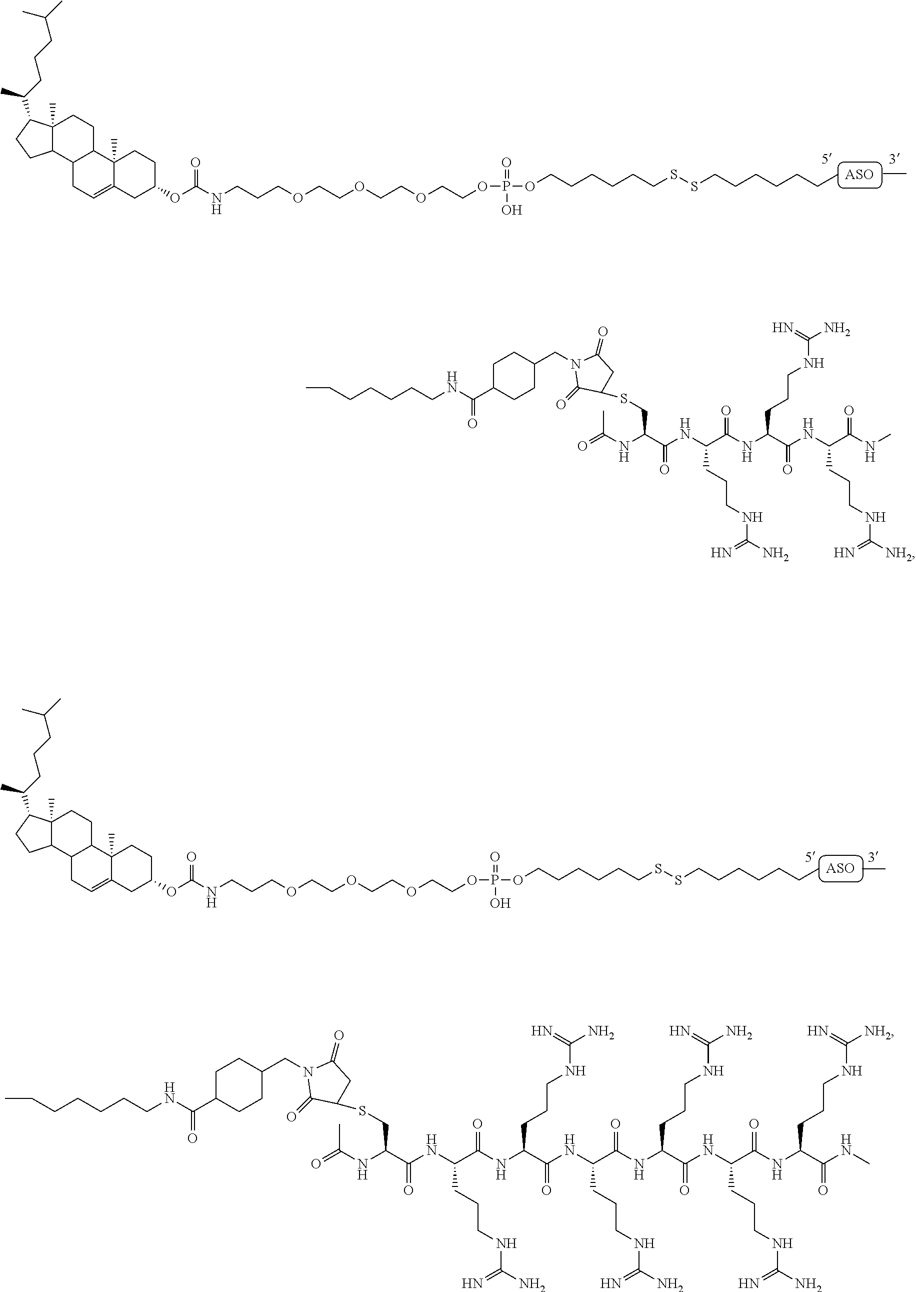

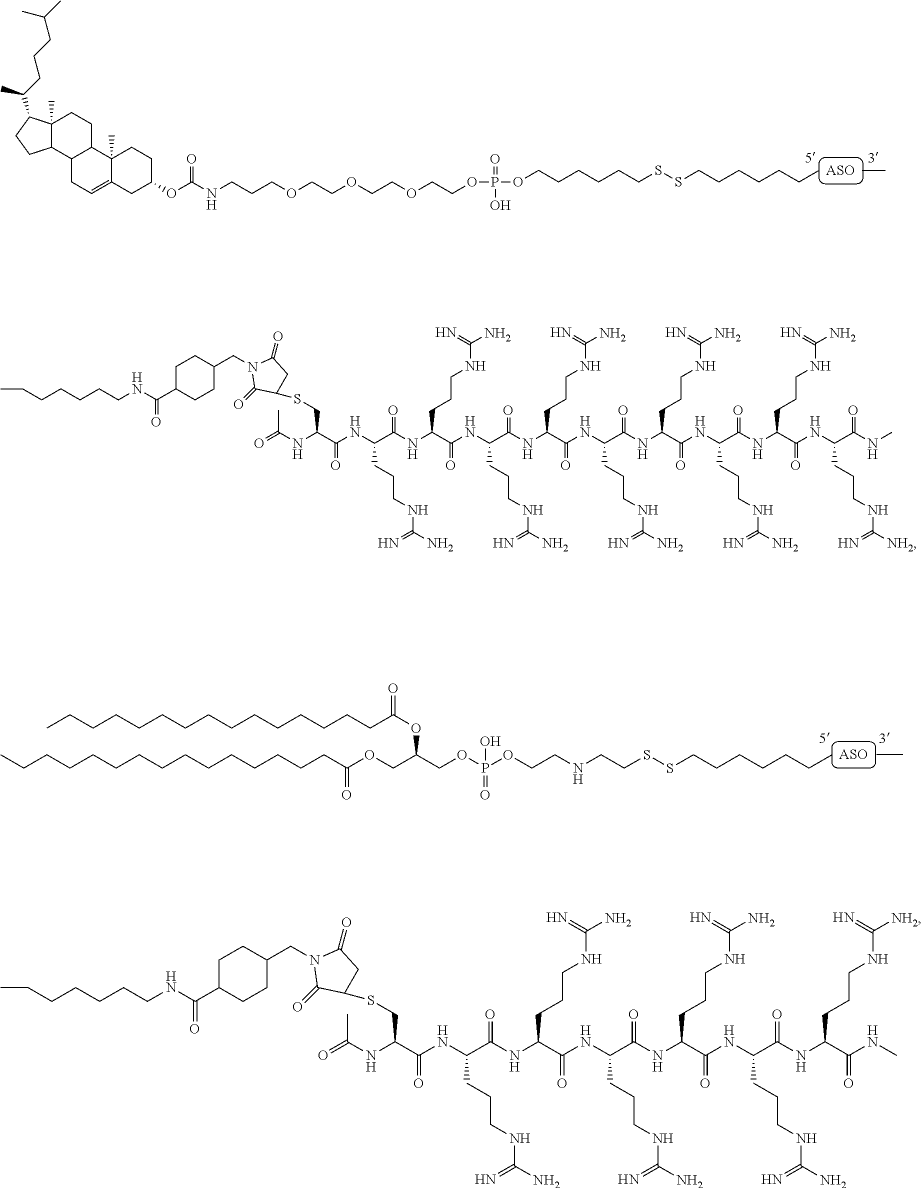

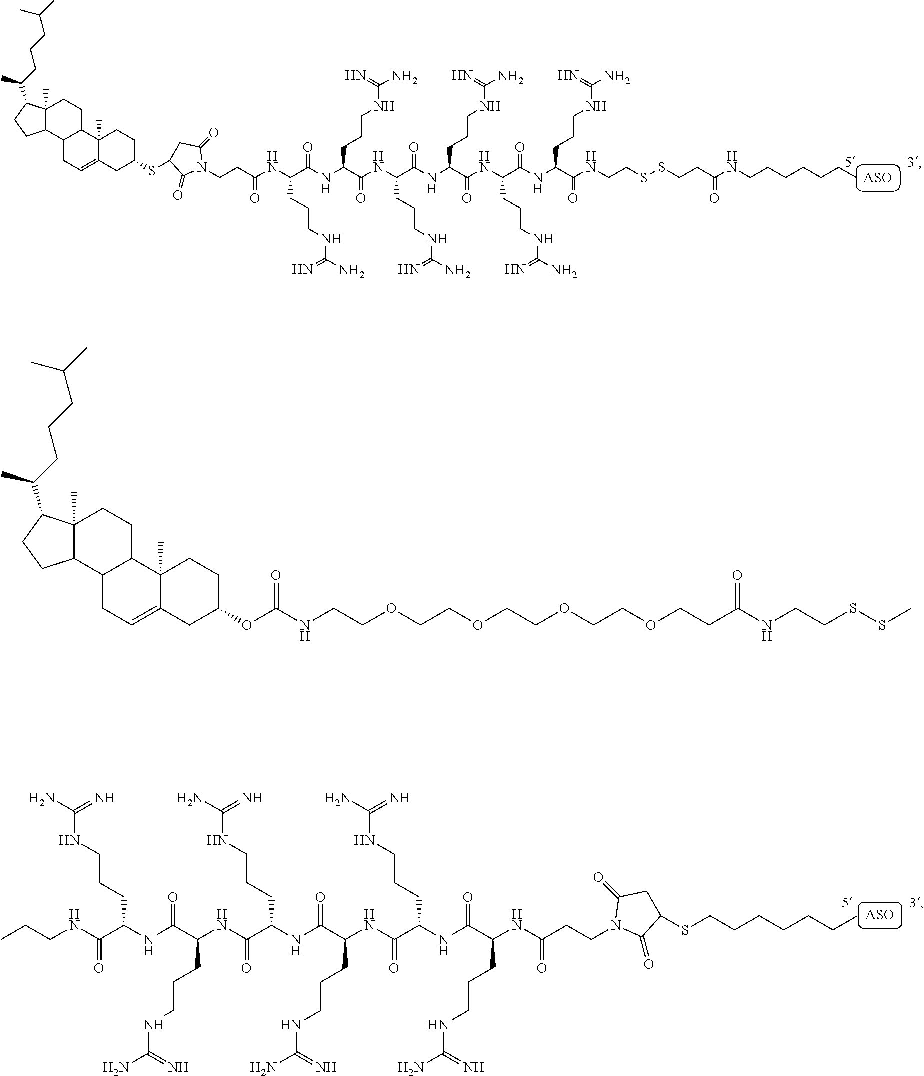

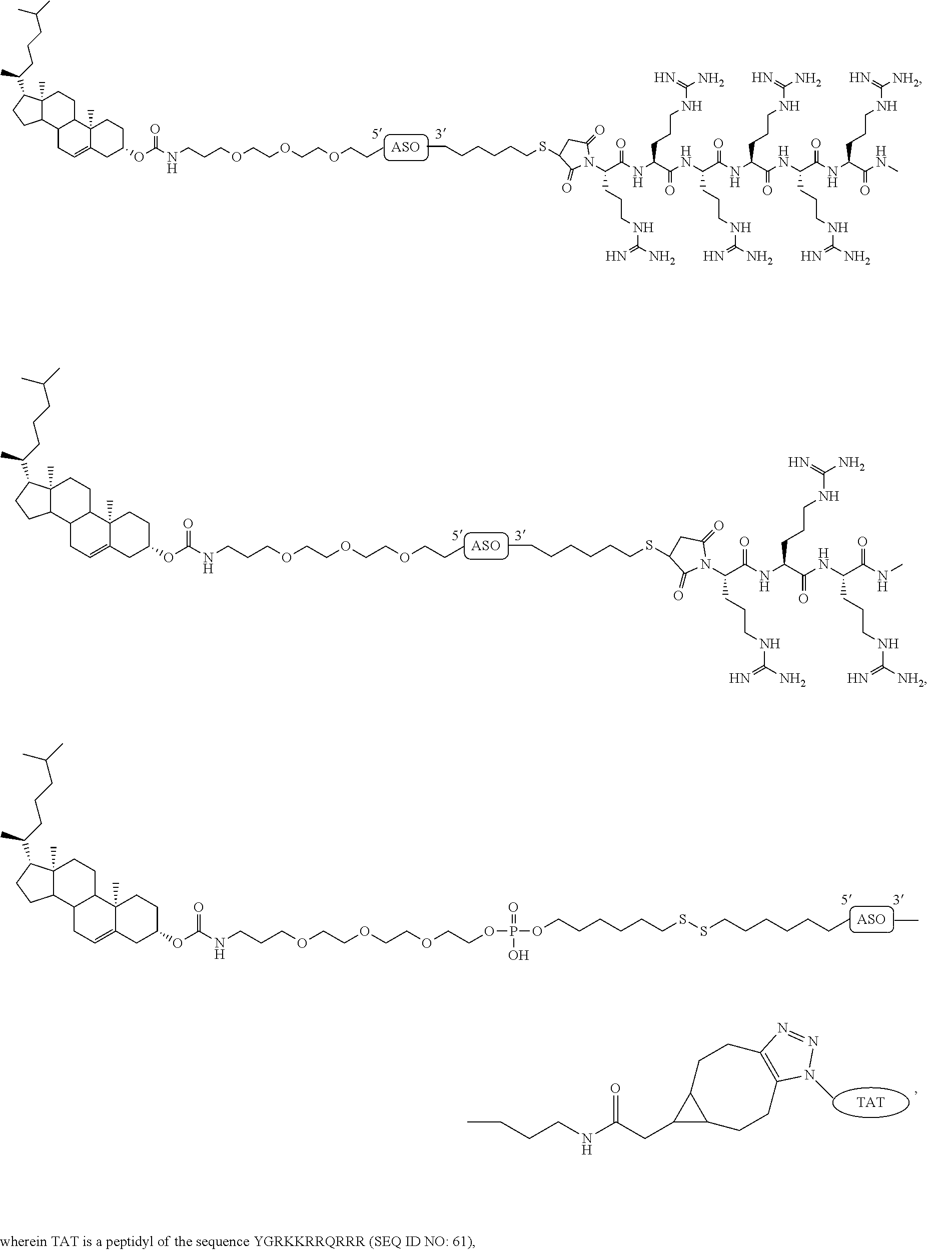

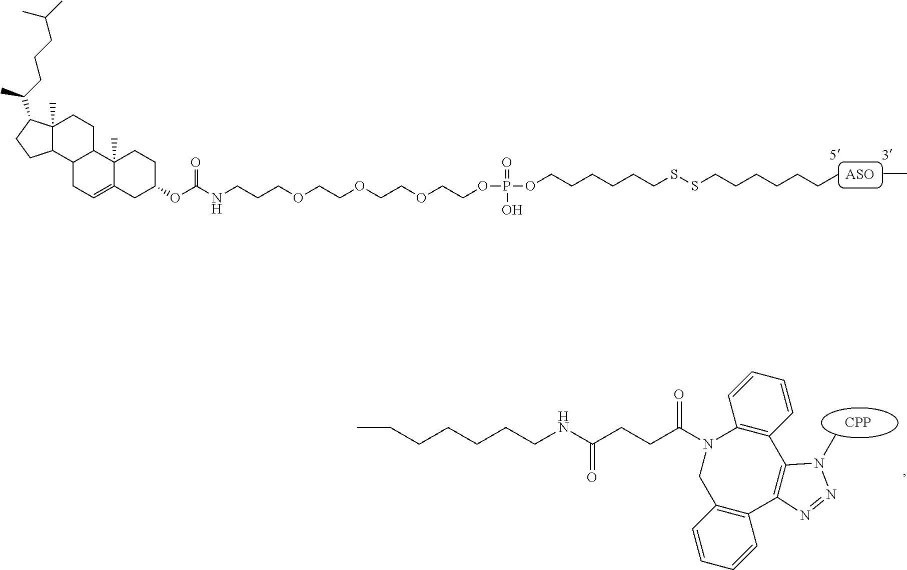

wherein the cell-penetrating peptide (CPP) is Antp (a peptidyl of the sequence RQIKIWFQNRRMKWKK (SEQ ID NO: 62)), R6 (a peptidyl of the sequence RRRRRR (SEQ ID NO: 87)), or cTAT (a peptide of sequence KRRRGRKKRRE (wherein K and E are connected to form a cyclic peptide)(SEQ ID NO: 88)), and

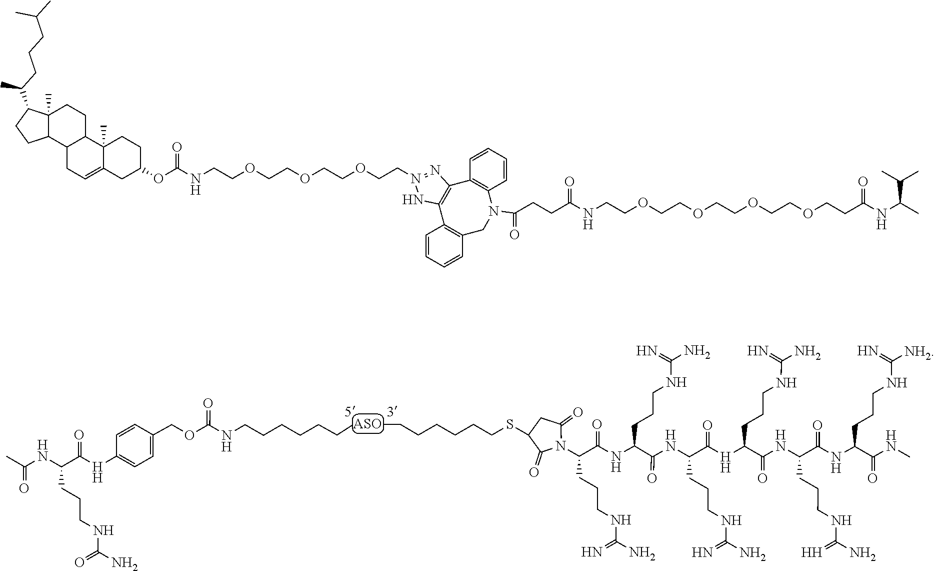

The present disclosure provides a pharmaceutical composition comprising the EV described herein and a pharmaceutically acceptable carrier.

The present disclosure also provides a kit comprising the EV described herein or a pharmaceutical composition thereof and instructions for use.

The present disclosure provides a method of treating or preventing a disease or disorder in a subject in need thereof comprising administering an effective amount of the EV described herein or a pharmaceutical composition thereof to the subject. In some aspects, the disease or disorder is a cancer, graft-versus-host disease (GvHD), an autoimmune disease, an infectious disease, a fibrotic disease, an inflammatory disease, a neurodegenerative disorder, a central nervous disease, a muscular dystrophy disease, or a metabolic disease.

### BRIEF DESCRIPTION OF THE DRAWINGS/FIGURES

FIG. 1 is a schematic view showing the general structure of an exosome (left), an exemplary biologically active molecule (e.g., an oligonucleotide) connected to a ligand that allows the attachment to the exterior surface of the exosome via a linker (center), and how the biologically active molecule (e.g., an oligonucleotide) connected to an anchoring moiety (e.g., a lipid, such as cholesterol) via a linker can be attached to the membrane of the exosome (right).

FIG. 2 shows the sequences of exemplary ASOs. FFLUC and RLUC are named after the luminescent reporter genes targeted. The MYC and STAT6 ASO are named after the genes targeted by the ASO. Nb: LNA residues (including LNA-5MeC and LNA T/LNA-5MeU). Nm: 2′-O′MOE residues (including MOE-5MeC and MOE-T/MOED-5MeU). dN: DNA residues. (5MdC): 5-Methyl-dC. s: phosphonothioate backbone modification.

FIG. 3 is a table of some properties of lipid-linker-ASO stock solutions.

FIG. 4 is a bar graph of the average diameters (nm) of reconstituted lipid-linker-ASO (-CPP) after filtration by a 0.2 μm filter.

FIG. 5 is a table of some properties of loaded ASO concentrations.

FIG. 6 is a bar graph of the loading density of exemplary exoASOs, which represents the number of ASOs loaded per number of exosomes.

FIG. 7 is graph of the IC₅₀ values (nm) of exemplary exoASOs.

FIG. 8 is a graph of IC₅₀ values (nm) of exemplary exoASOs normalized to the loading density. The “*” means that the ASO was loaded under unoptimized loading conditions, such that the loading density has the potential of further improvement.

FIG. 9 is a table of some properties of exoASO, including loaded ASO concentrations and IC₅₀.

FIG. 10 is a graph of percent gene expression (hSTAT6) normalized to ASO concentration (nM) of various exoASOS for ASO numbers 1 to 11.

FIG. 11 is a graph of percent gene expression (hSTAT6) normalized to exo concentration of various exoASOs for ASO numbers 1 to 11.

FIG. 12 is a graph of IC₅₀ comparison normalized to ASO concentration (nM) for ASO numbers 1 to 11.

FIG. 13 is a graph of IC₅₀ comparison normalized to exosome concentration (p/mL) for ASO numbers 1 to 11.

FIG. 14 is a graph of mSTAT6 knockdown (KD) in mouse liver with a single dose of exoASO (5 or 10 μg dose based on ASO weight) with various cleavable linkers and cell penetrating pepetide (CPP).

### DETAILED DESCRIPTION

The present disclosure is directed to extracellular vesicles (EVs), e.g., exosomes, comprising at least one biologically active molecule covalently linked to the EV, e.g., exosome, via a cleavable linker and an anchoring moiety and uses thereof. Non-limiting examples of the various aspects are shown in the present disclosure.

Before the present disclosure is described in greater detail, it is to be understood that this invention is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C(alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “agonist” refers to a molecule that binds to a receptor and activates the receptor to produce a biological response. Receptors can be activated by either an endogenous or an exogenous agonist. Non-limiting examples of endogenous agonist include hormones, neurotransmitters, and cyclic dinucleotides. Non-limiting examples of exogenous agonist include drugs, small molecules, and cyclic dinucleotides. The agonist can be a full, partial, or inverse agonist.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An (YZ), wherein A is the single letter code corresponding to the amino acid residue substituting the amino acid naturally or originally present at position n, and Y and Z are alternative substituting amino acid residues that can replace A.

As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. Non-limiting examples of antagonists include alpha blockers, beta-blocker, and calcium channel blockers. The antagonist can be a competitive, non-competitive, or uncompetitive antagonist.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv) 2, Fab, Fab′, and F (ab′)₂, F (ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “biologically active molecule” as use herein refers to any molecule that can be attached to an EV, e.g., exosome, via an anchoring moiety, wherein the molecule can have a therapeutic or prophylactic effect in a subject in need thereof, or be used for diagnostic purposes. Accordingly, by way of example, the term biologically active molecule includes proteins (e.g., antibodies, proteins, polypeptides, and derivatives, fragments, and variants thereof), lipids and derivatives thereof, carbohydrates (e.g., glycan portions in glycoproteins), or small molecules (e.g., a molecule with a molecular weight of 1000 g/mol or less). In some aspects, the biologically active molecule includes a radioisotope. In some aspects, the biologically active molecule is a detectable moiety, e.g., a radionuclide, a fluorescent molecule, or a contrast agent. In some aspects, the biologically active molecule can be or can comprise a targeting moiety or a tropism moiety. In some aspects, the biologically active molecule can be or can comprise, for example, an affinity ligand (e.g., biotin, digoxigenin, or dinitrophenol). In some aspects, the biologically active molecule can be or can comprise a moiety capable of improving a pharmacokinetic or pharmacodynamic property, for example, a moiety capable in increasing the plasma half-life, e.g., a PEG moiety.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are about 70% identical or higher, e.g., about 80% identical, about 90% identical, about 95%, about 98%, or about 99% identical to one another. In some aspects, two or more sequences are said to be “conserved” if they are up to about 70% identical, including about 30% identical, about 40% identical, about 50% identical, about 60% identical, or about 65% identical, to one another. Conservation of sequence can apply to the entire length of a polynucleotide or polypeptide or can apply to a portion, region, or feature thereof.

As used herein, the term “conventional EV protein” means a protein previously known to be enriched in EVs.

As used herein, the term “conventional exosome protein” means a protein previously known to be enriched in exosomes, including but not limited to CD9, CD63, CD81, PDGFR, GPI anchor proteins, lactadherin LAMP2, and LAMP2B, a fragment thereof, or a peptide that binds thereto.

The term “derivative” as used herein refers to an EV, e.g., exosome, component (e.g., a protein, such as Scaffold X, a lipid, or a carbohydrate) or to a biologically active molecule (e.g., a polypeptide, polynucleotide, lipid, carbohydrate, antibody or fragment thereof, PROTAC, etc.) that has been chemically modified to introduce at least one reactive moiety (e.g., a phosphoramidite moiety).

The terms “excipient” and “carrier” are used interchangeably and refer to an inert substance added to a pharmaceutical composition to further facilitate administration of a compound or EV.

As used herein, the terms “extracellular vesicle,” “EV,” and grammatical variants thereof, are used interchangeably and refer to a cell-derived vesicle comprising a membrane that encloses an internal space. Extracellular vesicles comprise all membrane-bound vesicles (e.g., an exosome, a microvesicle, a nanovesicle, an ectosome, an oncosome, or an apoptotic body) that have a smaller diameter than the cell from which they are derived. In some aspects, extracellular vesicles range in diameter from 20 nm to 1000 nm (e.g., 50 nm to 1000 nm, 50 nm to 200 nm, or 200 nm to 1000 nm), and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., 40-200 nm, 50-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the exosomes of the present disclosure are produced by cells that express one or more transgene products.

In some aspects, EVs, e.g., exosomes, e.g., nanovesicles, of the present disclosure are engineered by covalently linking at least one biologically active molecule (e.g., a protein such as an antibody or antibody drug conjugate (ADC), a ribonucleic acid (RNA) or deoxyribonucleic acid (DNA), such as an antisense oligonucleotide, a small molecule drug, or a small molecule toxin) to the EV, e.g., exosome or nanovesicle, via an anchoring moiety.

In some aspects, the EVs, e.g., exosomes or nanovesicles, of the present disclosure can comprise various macromolecular payloads either within the internal space (i.e., lumen), displayed on the external (exterior) surface or internal (luminal) surface of the EV, and/or spanning the membrane. In some aspects, the payload can comprise, e.g., nucleic acids, proteins, carbohydrates, lipids, small molecules, and combinations thereof. In certain aspects, an EV, e.g., an exosome, comprises a scaffold moiety (e.g., Scaffold X). EVs, e.g., exosomes, can be derived from a living or a dead organism, explanted tissues or organs, prokaryotic or eukaryotic cells, and/or cultured cells. In some aspects, the EVs, e.g., exosomes, are produced by cells that express one or more transgene products. In other aspects, the EVs of the present disclosure are without limitation nanovesicles, microsomes, microvesicles, extracellular bodies, or apoptotic bodies.

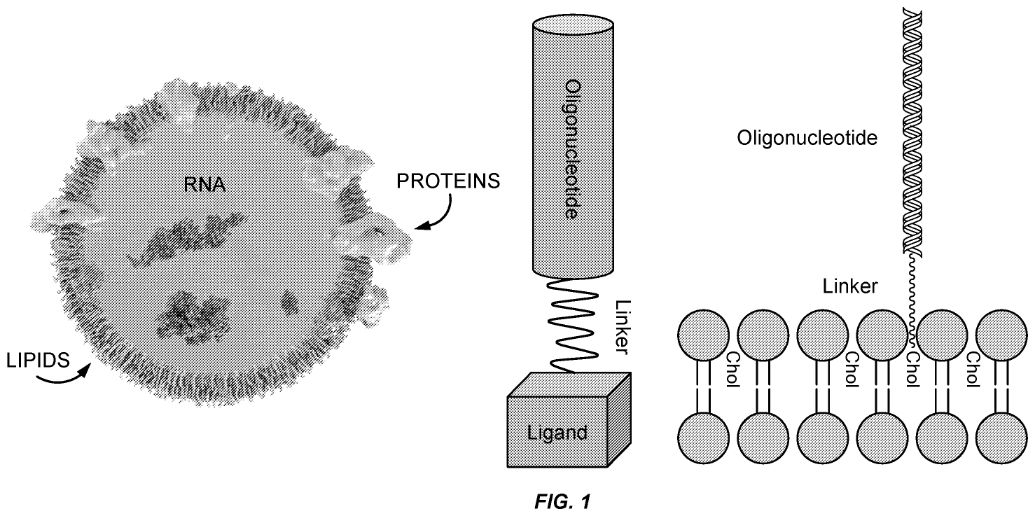

A schematic view of the general structure of an exosome, an exemplary biologically active molecule (e.g., an oligonucleotide) connected to a ligand (e.g., anchoring moiety) that allows the attachment to the exterior surface of the exosome via a linker, and how the biologically active molecule (e.g., an oligonucleotide) connected to an anchoring moiety (e.g., a lipid, such as cholesterol) via a linker can be attached to the membrane of the exosome, are shown in FIG. 1. In some aspects, the AM is attached to an exterior surface of the EV.

As used herein, the term “fragment” of a protein (e.g., a biologically active molecule such as a therapeutic protein, or a scaffold protein such as Scaffold X) refers to an amino acid sequence of a protein that is shorter than the naturally-occurring sequence, N- and/or C-terminally deleted or any part of the protein deleted in comparison to the naturally occurring protein.

As used herein, the term “functional fragment” refers to a protein fragment that retains protein function. Accordingly, in some aspects, a functional fragment of a Scaffold protein, e.g., Scaffold X protein, retains the ability to anchor a biologically active molecule on the luminal surface or on the external surface of the EV, e.g., exosome.

Whether a fragment is a functional fragment can be assessed by any art known methods to determine the protein content of EVs, e.g., exosomes, including Western Blots, fluorescence activated cell sorting (FACS) analysis and fusions of the fragments with autofluorescent proteins like, e.g., green fluorescent protein (GFP). In certain aspects, a functional fragment of a Scaffold X protein retains, e.g., at least 50%, at least 60%, at least 70%, at least 80%, at least 90%, at least 95%, or about 100% of the ability of the naturally occurring Scaffold X protein to anchor a biologically active molecule on the luminal or on the external surface of the EV, e.g., exosome.

As used herein “anchoring” a biologically active molecule on the luminal or external surface of an EV (e.g., exosome) of the present disclosure via a scaffold protein refers to attaching covalently or non-covalently the biologically active molecule to the portion of the scaffold molecule located on a luminal or external surface of the EV (e.g., exosome), respectively.

As used herein, the term “homology” refers to the overall relatedness between polymeric molecules, e.g., between nucleic acid molecules (e.g., DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polypeptide molecules or polynucleotide molecules (e.g., DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is b12seq, part of the BLAST (Basic Local Alignment Search Tool) suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT (multiple alignment using fast Fourier Transform), Clustal (ClustalW, Clustal X or Clustal Omega), MUSCLE (multiple sequence comparison by log expectation), etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired EVs (e.g., a plurality of EVs of known or unknown amount and/or concentration), that has undergone one or more processes of purification, e.g., a selection or an enrichment of the desired EV, e.g., exosome, preparation. In some aspects, isolating or purifying as used herein is the process of removing, partially removing (e.g., a fraction) of the EVs, e.g., exosomes, from a sample containing producer cells. In some aspects, an isolated EV, e.g., exosome, composition has no detectable undesired activity or, alternatively, the level or amount of the undesired activity is at or below an acceptable level or amount. In other aspects, an isolated EV, e.g., exosome, composition has an amount and/or concentration of desired EVs, e.g., exosomes, at or above an acceptable amount and/or concentration. In other aspects, the isolated EVs, e.g., exosome, composition is enriched as compared to the starting material (e.g., producer cell preparations) from which the composition is obtained. This enrichment can be by at least 10%, at least 20%, at least 30%, at least 40%, at least 50%, at least 60%, at least 70%, at least 80%, at least 90%, at least 95%, at least 96%, at least 97%, at least 98%, at least 99%, at least 99.9%, at least 99.99%, at least 99.999%, at least 99.9999%, or greater than 99.9999% as compared to the starting material. In some aspects, isolated EV, e.g., exosome, preparations are substantially free of residual biological products. In some aspects, the isolated EV, e.g., exosome, preparations are 100% free, at least 99% free, at least 98% free, at least 97% free, at least 96% free, at least 95% free, at least 94% free, at least 93% free, at least 92% free, at least 91% free, or at least 90% free of any contaminating biological matter. Residual biological products can include abiotic materials (including chemicals) or unecessary nucleic acids, proteins, lipids, and/or metabolites. Substantially free of residual biological products can also mean that the EV, e.g., exosome, composition contains no detectable producer cells and that only EVs, e.g., exosomes, are detectable.

The terms “linked,” “fused,” and grammatical variants thereof are used interchangeably and refer to a first moiety, e.g., a first amino acid sequence or nucleotide sequence, covalently or non-covalently joined to a second moiety, e.g., a second amino acid sequence, nucleotide sequence, and/or a lipid (e.g., cholesterol), respectively. The first moiety can be directly joined or juxtaposed to the second moiety or alternatively an intervening moiety can covalently join the first moiety to the second moiety. The term “linked” means not only a fusion of a first moiety to a second moiety at the C-terminus or the N-terminus, but also includes insertion of the whole first moiety (or the second moiety) into any two points, e.g., amino acids, in the second moiety (or the first moiety, respectively). In one aspect, the first moiety is linked to a second moiety by a peptide bond or a linker. The first moiety can be linked to a second moiety by a phosphodiester bond or a linker. The linker can be a peptide or a polypeptide (for polypeptide chains) or a nucleotide or a nucleotide chain (for nucleotide chains) or any chemical moiety (for polypeptide or polynucleotide chains or any chemical molecules). The term “linked” is also indicated by a hyphen (-). In some aspects, a Scaffold X protein on an EV, e.g., exosome, can be linked or fused to a biologically active molecule via a linker, a spacer, or both a linker and a spacer.

The term “modified,” when used in the context of EVs, e.g., exosomes, described herein, refers to an alteration or engineering of an EV, e.g., exosome and/or its producer cell, such that the modified EV, e.g., exosome, is different from a naturally-occurring EV, e.g., exosome. In some aspects, a modified EV, e.g., exosome, described herein comprises a membrane that differs in composition of a protein, a lipid, a small molecule, a carbohydrate, etc. compared to the membrane of a naturally-occurring EV, e.g., exosome. In an aspect, the membrane comprises higher density or number of natural EV, e.g., exosome, proteins and/or membrane comprises proteins that are not naturally found in EV, e.g., exosomes. In certain aspects, such modifications to the membrane change the exterior surface of the EV, e.g., exosome (e.g., surface-engineered EVs and exosomes described herein).

As used herein the terms “modified protein” or “protein modification” refers to a protein having at least 15% identity to the non-mutant amino acid sequence of the protein. A modification of a protein includes a fragment or a variant of the protein. A modification of a protein can further include chemical or physical modification to a fragment or a variant of the protein.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety, e.g., Scaffold X. Nanovesicles, once derived from a producer cell, can be isolated from the producer cell based on its size, density, biochemical parameters, or a combination thereof.

As used herein, the term “payload” refers to a biologically active molecule (e.g., a therapeutic agent) that acts on a target (e.g., a target cell) that is contacted with the EV, e.g., exosome, of the present disclosure. Non-limiting examples of payloads that can be introduced into an EV, e.g., exosome, include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an antigen. As used herein, the term “antigen” refers to any agent that when introduced into a subject elicits an immune response (cellular or humoral) to itself. In some aspects, the payload molecules are covalently linked to the EV, e.g., exosome, via a linker, a spacer, or both a linker and a spacer, as disclosed herein. In other aspects, a payload comprises an adjuvant.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., an EV, such as exosome of the present disclosure, mixed or intermingled with, or suspended in one or more other chemical components, such as pharmaceutically-acceptable carriers and excipients. One purpose of a pharmaceutical composition is to facilitate administration of preparations of EVs, e.g., exosomes, to a subject.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded DNA, as well as triple-, double- and single-stranded RNA. It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure, the biologically active molecule attached to the EV, e.g., exosome, via a linker, a spacer, or both a linker and a space, as disclosed herein, is a polynucleotide, e.g., an antisense oligonucleotide. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide (e.g., an antisense oligonucleotide, ASO).

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV, e.g., exosome, via a linker, spacer, or both a linker and a spacer, as disclosed herein, is a polypeptide, e.g., an antibody or a derivative thereof such as an ADC, a proteolysis targeting chimera (PROTAC), a toxin, a fusion protein, or an enzyme.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The terms “prevent,” “preventing,” and variants thereof as used herein, refer to partially or completely delaying onset of a disease, disorder and/or condition; partially or completely delaying onset of one or more symptoms, features, or clinical manifestations of a particular disease, disorder, and/or condition; partially or completely delaying progression from a particular disease, disorder and/or condition; and/or decreasing the risk of developing pathology associated with a disease, disorder, and/or condition. In some aspects, preventing an outcome is achieved through prophylactic treatment.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN™ neuronal precursor cells, CAP™ amniocyte cells, adipose mesenchymal stem cells, RPTEC/TERTI cells. In certain aspects, a producer cell is not an antigen-presenting cell. In some aspects, a producer cell is not a dendritic cell, a B cell, a mast cell, a macrophage, a neutrophil, Kupffer-Browicz cell, a cell derived from any of these cells, or any combination thereof.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized. In some aspects of the present disclosure, the Scaffold X proteins present in EVs, e.g., exosomes, are recombinantly produced by overexpressing the scaffold proteins in the producer cells, so that levels of scaffold proteins in the resulting EVs, e.g., exosomes, are increased with respect to the levels of scaffold proteins present in EVs, e.g., exosomes, of producer cells not overexpressing such scaffold proteins.

As used herein, the term “scaffold moiety” refers to a molecule, e.g., a protein such as Scaffold X, that can be used to anchor a payload, e.g., a biologically active molecule, to the EV, e.g., exosome, e.g., on the external surface of the EV. In certain aspects, a scaffold moiety comprises a synthetic molecule. In some aspects, a scaffold moiety comprises a non-polypeptide moiety. In other aspects, a scaffold moiety comprises, e.g., a lipid, carbohydrate, protein, or combination thereof (e.g., a glycoprotein or a proteolipid) that naturally exists in the EV, e.g., exosome. In some aspects, a scaffold moiety comprises a lipid, carbohydrate, or protein that does not naturally exist in the EV, e.g., exosome. In some aspects, a scaffold moiety comprises a lipid or carbohydrate which naturally exists in the EV, e.g., exosome, but has been enriched in the EV, e.g., exosome with respect to basal/native/wild type levels. In some aspects, a scaffold moiety comprises a protein which naturally exists in the EV, e.g., exosome but has been enriched in the EV, e.g., exosome, for example, by recombinant overexpression in the producer cell, with respect to basal/native/wild type levels. In certain aspects, a scaffold moiety is Scaffold X.

As used herein, the term “Scaffold X” refers to EV, e.g., exosome, proteins that have been identified on the surface of EVs, e.g., exosomes. See, e.g., U.S. Pat. No. 10,195,290, which is incorporated herein by reference in its entirety. Non-limiting examples of Scaffold X proteins include: prostaglandin F2 receptor negative regulator (“PTGFRN”); basigin (“BSG”); immunoglobulin superfamily member 2 (“IGSF2”); immunoglobulin superfamily member 3 (“IGSF3”); immunoglobulin superfamily member 8 (“IGSF8”); integrin beta-1 (“ITGB1”); integrin alpha-4 (“ITGA4”); 4F2 cell-surface antigen heavy chain (“SLC3A2”); and a class of ATP transporter proteins (“ATP1A1,” “ATPIA2,” “ATPIA3,” “ATPIA4,” “ATPIB3,” “ATP2B1,” “ATP2B2,” “ATP2B3,” “ATP2B”). In some aspects, a Scaffold X protein can be a whole protein or a fragment thereof (e.g., functional fragment, e.g., the smallest fragment that is capable of anchoring another moiety on the external surface or on the luminal surface of the EV, e.g., exosome). In some aspects, a Scaffold X can anchor a biologically active molecule to the external surface or the lumen of the EV, e.g., an exosome. In some aspects of the present disclosure, a biologically active molecule can be covalently attached to a Scaffold X, e.g., via a linker, spacer, or both a linker and a spacer, as disclosed herein. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

As used herein, the term “small molecule” refers to an organic compound with a molecular weight of about 1000 g/mol or less (e.g., about 900 g/mol or less) enabling passage through a cell membrane. The organic compound can be, e.g., a biological molecule, a drug or a toxin. Suitable biological molecules include, e.g., a cyclic dinucleotide, a fatty acid, a sugar (e.g., glucose), an amino acid, a lipid (e.g., cholesterol), a phenolic compound, and an alkaloid. Suitable drugs include, e.g., analgesics, antibacterial agents, antiviral agents, anticonvulsants, antipsychotics, antineoplastics, anti-inflammatory agents, anti-obesity agents, antiparsitics, contraceptives, otic agents, ophthalmic agents, skeletal muscle relaxants, sleep disorder agents, central nervous system agents, cardiovascular agents, blood glucose regulator, immunological agents, infertility agents, respiratory tract agents, tyrosine kinase inhibitors, and mTOR inhibitors. Specific examples include but are not limited to asprin, naproxen, celecoxib, diclofenac, ketorolac, oxycodone, insulin, methotrexate, sulfasalazine, imiquimod, cyclophosphamide, mycophenolate mofetil, marmastat, dimercaprol, doxorubicin, taxol, and paclitaxel. Suitable toxins include, e.g., bacterial toxins, incuding a hemotoxin, a phototoxin, a hepatotoxin, and a neurotoxin, a mycotoxin, an aflaxtoxin, an ochratoxin, a citrinin, and an ergot alkaloid. Specific exmaples include monomethyl auristatin E (MMAE) botulinum toxin A, tetanus toxin A, edema toxin, exotoxin A, cholera toxin, pertussis toxin, diphtheria toxin, dioxin, muscarine, and bufotoxin, or synthetic toxins, such as bisphenol A, perchlorate, tetrachloroethylene, 2-butoxyethanol, and formaldehyde.

Unless otherwise indicated, reference to a compound that has one or more stereocenters intends each stereoisomer, and all combinations of stereoisomers, thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the term “substantially free” means that the sample comprising EVs, e.g., exosomes, comprises less than 10% of macromolecules, e.g., contaminants, by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein the term “surface-engineered EV” (e.g., Scaffold X-engineered exosome) refers to an EV with the membrane or the surface of the EV modified in its composition so that the surface of the engineered EV is different from that of the EV prior to the modification or of the naturally occurring EV.

As used herein the term “surface-engineered exosome” (e.g., Scaffold X-engineered exosome) refers to an exosome with the membrane or the surface of the exosome (external surface or luminal surface) modified in its composition so that the surface of the engineered exosome is different from that of the exosome prior to the modification or of the naturally occurring exosome.

The engineering can be on the surface of the EV, e.g., exosome, or in the membrane of the EV, e.g., exosome, so that the surface of the EV, e.g., exosome, is changed. For example, the membrane can be modified in its composition of, e.g., a protein, a lipid, a small molecule, a carbohydrate, or a combination thereof. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome or can be an anchoring point (attachment) for a moiety exposed on the surface of the EV, e.g., exosome. In other aspects, a surface-engineered EV, e.g., exosome comprises a higher expression (e.g., higher number) of a natural EV, e.g., exosome protein (e.g., Scaffold X) or a fragment or variant thereof that can be exposed to the surface of the EV, e.g., exosome or can be an anchoring point (attachment) for a moiety exposed on the surface of the EV, e.g., exosome. In a specific aspect, a surface-engineered EV, e.g., exosome, comprises the modification of one or more membrane components, e.g., a protein such as Scaffold X, a lipid, a small molecule, a carbohydrate, or a combination thereof, wherein at least one of the components is covalently attached to a biologically active molecule via a linker, spacer, or both a linker and a spacer, as disclosed herein.

As used herein the term “therapeutically effective amount” is the amount of reagent or pharmaceutical compound comprising an EV or exosome of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition to any suitable degree, without necessarily curing the disease or condition. The term also includes prophylaxis or prevention of a disease or condition or its symptoms thereof. In one aspect, the term “treating” or “treatment” means inducing an immune response in a subject against an antigen.

As used herein, the term “variant” of a molecule (e.g., functional molecule, antigen, or Scaffold X) refers to a molecule that shares certain structural and functional identities with another molecule upon comparison by a method known in the art. For example, a variant of a protein can include a substitution, insertion, deletion, frame shift, or rearrangement in another protein.

In some aspects, a variant of a Scaffold X or derivative comprises a Scaffold X variant having at least 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins.

In some aspects, the variant or variant of a fragment of Scaffold X protein disclosed herein, or derivatives thereof, retains the ability to be specifically targeted to EVs, e.g., exosomes. In some aspects, the Scaffold X or Scaffold X derivative includes one or more mutations, for example, conservative amino acid substitutions.

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268:2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m] ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, variants or derivatives include, e.g., modified polypeptides. In some aspects, variants or derivatives of, e.g., polypeptides, polynucleotides, lipids, glycoproteins, are the result of chemical modification and/or endogenous modification. In some aspects, variants or derivatives are the result of in vivo modification. In some aspects, variants or derivatives are the result of in vitro modification. In yet other aspects, variant or derivatives are the result of intracellular modification in producer cells.

Modifications present in variants and derivatives include, e.g., acetylation, acylation, adenosine diphosphate ribose (ADP) ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination.

In some aspects, Scaffold X can be modified at any convenient location. In some aspects, a biologically active molecule can be modified at any convenient location. In particular aspects of the present disclosure, an EV, e.g., exosome, component (e.g., a protein such as Scaffold X, a lipid, or a glycan) and/or a biologically active molecule (e.g., an antibody or ADC, a PROTAC, a small molecule such as a cyclic dinucleotide, a toxin such as MMAE, a STING (stimulator of interferon genes) agonist (e.g., CAS Nos. 702662-50-8 and 849214 Apr. 6), a tolerizing agent (e.g., agents disclosed in U.S. Pat. No. 7,910,113 and U.S. Patent Application Publication 2009/0169578), or an antisense oligonucleotide) can be modified to yield a derivative comprising at least one linker, spacer, or both a linker and a spacer, as disclosed herein.

The term “alkyl,” by itself or as part of another substituent, means, unless otherwise stated, a straight or branched chain hydrocarbon radical having the number of carbon atoms designated (e.g., C₁-C₁₀ means one to ten carbon atoms). Typically, an alkyl group will have from 1 to 15 carbon atoms, for example, having from 1 to 10 carbon atoms, from 1 to 8 carbon atoms or from 1 to 6 carbon atoms. A “lower alkyl” group is an alkyl group having from 1 to 4 carbon atom (e.g., 1 to 3 carbon atoms or 1 to 2 carbon atoms). The term “alkyl” includes mono-, di-, and multivalent radicals. For example, the term “alkyl” includes “alkylenyl” wherever appropriate, e.g., when the formula indicates that the alkyl group is divalent or when substituents are joined to form a ring. Examples of alkyl radicals include, but are not limited to, methyl, ethyl, n-propyl, isopropyl, n-butyl, tert-butyl, iso-butyl, sec-butyl, as well as homologs and isomers of, for example, n-pentyl, n-hexyl, n-heptyl and n-octyl.

The term “alkylenyl” by itself or as part of another substituent means a divalent (diradical) alkyl group, wherein alkyl is defined herein. “Alkylenyl” is exemplified, but not limited, by —CH₂CH₂CH₂CH₂—. Typically, an “alkylenyl” group will have from 1 to 15 carbon atoms, for example, having 10 or fewer carbon atoms (e.g., 1 to 8 or 1 to 6 carbon atoms). A “lower alkylenyl” group is an alkylene group having from 1 to 4 carbon atoms (e.g., 1 to 3 carbon atoms or 1 to 2 carbon atoms).

The term “alkenyl” by itself or as part of another substituent refers to a straight or branched chain hydrocarbon radical having from 2 to 15 carbon atoms and at least one double bond. A typical alkenyl group has from 2 to 10 carbon atoms and at least one double bond. In one aspect, alkenyl groups have from 2 to 8 carbon atoms or from 2 to 6 carbon atoms and from 1 to 3 double bonds. Exemplary alkenyl groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-5-enyl and the like.

The term “alkynyl” by itself or as part of another substituent refers to a straight or branched chain, unsaturated or polyunsaturated hydrocarbon radical having from 2 to 15 carbon atoms and at least one triple bond. A typical “alkynyl” group has from 2 to 10 carbon atoms and at least one triple bond. In one aspect of the disclosure, alkynyl groups have from 2 to 6 carbon atoms and at least one triple bond. Exemplary alkynyl groups include prop-1-ynyl, prop-2-ynyl (i.e., propargyl), ethynyl and 3-butynyl.

The terms “alkoxy,” “alkylamino,” and “alkylthio” (or thioalkoxy) are used in their conventional sense, and refer to alkyl groups that are attached to the remainder of the molecule via an oxygen atom, an amino group, or a sulfur atom, respectively.

The term “heteroalkyl,” by itself or in combination with another term, means a stable, straight or branched chain hydrocarbon radical consisting of the stated number of carbon atoms (e.g., C₂-C₁₀, or C₂-C₈) and at least one heteroatom chosen, e.g., from N, O, S, Si, B, and P (in one aspect, N, O, and S), wherein the nitrogen, sulfur and phosphorus atoms are optionally oxidized, and the nitrogen atom(s) are optionally quaternized. The heteroatom(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₃—S—CH₂—CH₃, —CH₂—CH₂—S(O)—CH₃, —CH₂—CH₂—S(O)₂—CH₃, —CH═CH—O—CH₃, —CH₂—Si (CH₃) 3, —CH₂—CH═N—OCH₃, and —CH═CH—N(CH₃)—CH₃. Up to two heteroatoms can be consecutive, such as, for example, —CH₂—NH—OCH₃ and —CH₂—O—Si (CH₃)₃.

Similarly, the term “heteroalkylenyl” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen)(3- to 24-membered heteroalkyl). In another example, the heteroalkyl group has a total of 3 to 10 atoms (3- to 10-membered heteroalkyl) or from 3 to 8 atoms (3- to 8-membered heteroalkyl). The term “heteroalkyl” includes “heteroalkylenyl” wherever appropriate, e.g., when the formula indicates that the heteroalkyl group is divalent or when substituents are joined to form a ring.

The term “C₁₋₈ alkyl” as used herein refers to a straight chain or branched, saturated hydrocarbon having from 1 to 8 (e.g., 1 to 7, 1 to 6, 1 to 5, 1 to 4, 1 to 3, or 1 to 2) carbon atoms. Representative “C₁₋₈ alkyl” groups include, but are not limited to, methyl, ethyl, n-propyl, n-butyl, n-pentyl, n-hexyl, n-heptyl, n-octyl, isopropyl, sec-butyl, isobutyl, tert-butyl, isopentyl, and 2-methylbutyl.

The term “C₁₋₁₀ alkylenyl” refers to a saturated, straight chain hydrocarbon group of the formula —(CH₂)₁₋₁₀—. Examples of C₁₋₁₀ alkylenyl include methylenyl, ethylenyl, propylenyl, butylenyl, pentylenyl, hexylenyl, heptylenyl, octylenyl, nonylenyl, and decalenyl.

The term “cycloalkyl” by itself or in combination with other terms, represents a saturated or unsaturated, non-aromatic carbocyclic radical having from 3 to 24 carbon atoms, for example, having from 3 to 12 carbon atoms (e.g., C₃-C₈ cycloalkyl or C₃-C₆ cycloalkyl). Examples of cycloalkyl include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl and the like. The term “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3) other ring selected from aryl (e.g., phenyl), heteroaryl (e.g., pyridyl) and non-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The term “heterocycloalkyl,” “heterocyclic,” “heterocycle,” or “heterocyclyl,” by itself or in combination with other terms, represents a carbocyclic, non-aromatic ring (e.g., 3- to 8-membered ring and for example, 4-, 5-, 6- or 7-membered ring) containing at least one and up to 5 heteroatoms selected from, e.g., N, O, S, Si, B, and P (in an aspect, N, O, and S), wherein the nitrogen, sulfur and phosphorus atoms are optionally oxidized, and the nitrogen atom(s) are optionally quaternized (e.g., from 1 to 4 heteroatoms selected from nitrogen, oxygen and sulfur), or a fused ring system of 4- to 8-membered rings, containing at least one and up to 10 heteroatoms (e.g., from 1 to 5 heteroatoms selected from N, O and S) in stable combinations known to those of skill in the art. Exemplary heterocycloalkyl groups include a fused phenyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule.

Exemplary heterocycloalkyl or heterocyclic groups of the present disclosure include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperidinyl, tetrahydrofuranyl, tetrahydrothienyl, piperidinyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazolyl, dihydropyridyl, dihydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

By “aryl” is meant a 5-, 6- or 7-membered, aromatic carbocyclic group having a single ring (e.g., phenyl) or being fused to other aromatic or non-aromatic rings (e.g., from 1 to 3 other rings). When the “aryl” group includes a non-aromatic ring (such as in 1,2,3,4-tetrahydronaphthyl) or heteroaryl group then the “aryl” group is bonded to the remainder of the molecule via an aryl ring (e.g., a phenyl ring). The aryl group is optionally substituted (e.g., with 1 to 5 substituents described herein). In one example, the aryl group has from 6 to 10 carbon atoms. Non-limiting examples of aryl groups include phenyl, 1-naphthyl, 2-naphthyl, anthracenyl, quinoline, indanyl, indenyl, dihydronaphthyl, fluorenyl, tetralinyl, benzo[d][1,3]dioxolyl or 6,7,8,9-tetrahydro-5H-benzo[a] cycloheptenyl. In one aspects, the aryl group is selected from phenyl, benzo[d][1,3]dioxolyl and naphthyl. The aryl group, in yet another aspect, is phenyl.

The term “arylenyl” refers to an aryl group which has two open valencies bonds and can be in the ortho, meta, or para configurations as shown in the following structures:

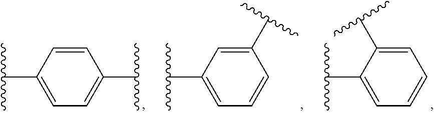

in which the arylenyl group can be unsubstituted or substituted with up to four (e.g., 1, 2, 3, or 4) groups including, but not limited to, C₁₋₈ alkyl, —O—(C₁₋₈ alkyl), -aryl, —C(O)R′, —OC(O)R′, —C(O) OR′, —C(O)NH₂, —C(O)NHR′, —C(O)N(R′)₂—, —NHC(O)R′, —S(O)₂R′, —S(O)R′, —OH, -halo, —N₃, —NH₂, —NH(R′), —N(R′)₂—NO₂, and —CN, wherein each R′ is independently H, —C₁₋₈ alkyl, or aryl.

The term “arylalkyl” or “aralkyl” is meant to include those radicals in which an aryl group or heteroaryl group is attached to an alkyl group to create the radicals-alkyl-aryl and -alkyl-heteroaryl, wherein alkyl, aryl and heteroaryl are defined herein. Exemplary “arylalkyl” or “aralkyl” groups include benzyl, phenethyl, pyridylmethyl and the like.

By “aryloxy” is meant the group-O-aryl, where aryl is as defined herein. In one example, the aryl portion of the aryloxy group is phenyl or naphthyl. The aryl portion of the aryloxy group, in one aspect, is phenyl.

The term “heteroaryl” or “heteroaromatic” refers to a polyunsaturated, 5-, 6- or 7-membered aromatic moiety containing at least one heteroatom (e.g., 1 to 5 heteroatoms, such as 1-3 heteroatoms) selected from N, O, S, Si, and B (in an aspect, N, O, and S), wherein the nitrogen and sulfur atoms are optionally oxidized, and the nitrogen atom(s) are optionally quaternized. The “heteroaryl” group can be a single ring or be fused to other aryl, heteroaryl, cycloalkyl or heterocycloalkyl rings (e.g., from 1 to 3 other rings). When the “heteroaryl” group includes a fused aryl, cycloalkyl or heterocycloalkyl ring, then the “heteroaryl” group is attached to the remainder of the molecule via the heteroaryl ring. A heteroaryl group can be attached to the remainder of the molecule through a carbon- or heteroatom.

In one example, the heteroaryl group has from 4 to 10 carbon atoms and from 1 to 5 heteroatoms selected from O, S and N. Non-limiting examples of heteroaryl groups include pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N—oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Exemplary heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Any of the organic residues described herein (e.g., alkyl, alkenyl, alkynyl, cycloalkyl, heterocycloalkyl, aryl, heteroaryl, etc.) can be unsubstituted or substituted with one or more (e.g., 1, 2, 3, 4, 5, 6, or 7) groups, as appropriate. Typical substituents include, but is not limited to, C₁₋₈ alkyl, —O—(C₁₋₈ alkyl), -aryl, —C(O)R′, —OC(O)R′, —C(O) OR′, —C(O)NH₂, —C(O)NHR′, —C(O)N(R′)₂—, —NHC(O)R′, —S(O) 2R′, —S(O)R′, —OH, -halo, —N₃, —NH₂, —NH (R′), —N(R′)₂ and —CN, wherein each R′ is independently H, —C₁₋₈ alkyl, or aryl.

### Extracellular Vesicles Comprising the Construct of Formula I

The present disclosure provides extracellular vesicle (EV) comprising a biologically active molecule (BAM) attached to the EV via an anchoring moiety (AM) according to Formula I or II:

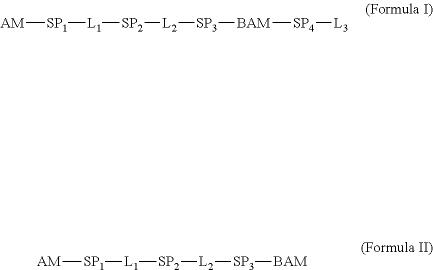

wherein
L₁, L₂, and L₃ are the same or different and each is an optional cleavable linkage; and SP₁, SP₂, SP₃, and SP₄ are optional first, second, third, and fourth spacers, respectively, wherein at least one of L₁, L₂, and L₃ is present and comprises a cell penetrating peptide.

As used herein the term “linker” refers to a combination of structural elements comprising, e.g., “linkages” (both cleavable and non-cleavable) and “spacers,” which connect an anchoring moiety AM and a biological active molecule BAM. These linkers allow loading biological active molecules (e.g., ASOs) more efficiently and in larger numbers onto the surface of EVs (e.g., exosomes) than a corresponding construct comprising the same anchoring moiety (AM) and biologically active molecule (BAM) in the absence of the cleavable linkage of the present disclosure. In other words, the constructs disclosed herein, e.g., a construct of Formula I or II, results in (i) higher EV loading efficiency, (ii) higher number of BAM per EV, (iii) higher density of BAM per EV, (iv) higher BAM potency, or (v) any combination thereof, with respect to a construct with the architecture AM-BAM or AM-SPI-BAM.

As used herein, the term “linkage” refers to any bond or chemical group connecting, e.g., an anchoring moiety AM and a spacer SP, a spacer SP and a biologically active molecule BAM, or an anchoring moiety AM and a biologically active molecule BAM. In constructs where more than an anchoring moiety AM or biologically active molecule BAM is present, a linkage can connect two anchoring moieties or two biologically active moieties. In some aspects, a “linkage” can be a bond that is cleavable, non-cleavable, or both cleavable and non-cleavable. In some aspects, a linkage can comprise multiple linkers and bonds, which can respond to different stimuli such as pH, temperature, enzymes, etc.

The term “spacer” as used herein refers to a chemical moiety which is capable of covalently linking together two spaced moieties (e.g., a biologically active molecule and an anchoring moiety) into a normally stable dipartate molecule. Generally, spacers as not cleavable. For example, a spacer can be an alkyl chain or a polyalkyloxy chain, as described herein.

In some aspects, the length of the linker connecting an anchoring moiety AM and a biologically active molecule BAM is between about 2 nm and about 30 nm. In some aspects, the length of the linker connecting an anchoring moiety AM and a biologically active molecule BAM is about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, or about 30 nm. In some aspects, the length of the optimized linker is at least 2 nm, at least 3 nm, at least 4 nm, at least 5 nm, at least 6 nm, at least 7 nm, at least 8 nm, at least 9 nm, at least 10 nm, at least 11 nm, at least 12 nm, at least 13 nm, at least 14 nm, at least 15 nm, at least 16 nm, at least 17 nm, at least 18 nm, at least 19 nm, at least 20 nm, at least 21 nm, at least 22 nm, at least 23 nm, at least 24 nm, at least 25 nm, at least 26 nm, at least 27 nm, at least 28 nm, at least 29 nm, or at least 30 nm. In some aspects, the length of the optimized linker is less than about 2 nm, less than about 3 nm, less than about 4 nm, less than about 5 nm, less than about 6 nm, less than about 7 nm, less than about 8 nm, less than about 9 nm, less than about 10 nm, less than about 11 nm, less than about 12 nm, less than about 13 nm, less than about 14 nm, less than about 15 nm, less than about 16 nm, less than about 17 nm, less than about 18 nm, less than about 19 nm, less than about 20 nm, less than about 21 nm, less than about 22 nm, less than about 23 nm, less than about 24 nm, less than about 25 nm, less than about 26 nm, less than about 27 nm, less than about 28 nm, less than about 29 nm, or less than about 30 nm.

In some aspects, the length of the linker connecting an anchoring moiety AM and a biologically active molecule BAM is about 2 nm to about 4 nm, about 3 nm to about 5 nm, about 4 nm to about 6 nm, about 5 nm to about 7 nm, about 6 nm to about 8 nm, about 7 nm to about 9 nm, about 8 nm to about 10 nm, about 9 nm to about 11 nm, about 10 nm to about 12 nm, about 11 nm to about 13 nm, about 12 nm to about 14 nm, about 13 nm to about 15 nm, about 14 nm to about 16 nm, about 15 nm to about 17 nm, about 16 nm to about 18 nm, about 17 nm to about 19 nm, about 18 nm to about 20 nm, about 19 nm to about 21 nm, about 20 nm to about 22 nm, about 21 nm to about 23 nm, about 22 nm to about 24 nm, about 23 nm to about 25 nm, about 24 nm to about 26 nm, about 25 nm to about 27 nm, about 26 nm to about 28 nm, about 27 nm to about 29 nm, about 28 nm to about 30 nm, about 2 nm to about 6 nm, about 4 nm to about 8 nm, about 6 nm to about 10 nm, about 8 nm to about 12 nm, about 10 nm to about 14 nm, about 12 nm to about 16 nm, about 14 nm to about 18 nm, about 16 nm to about 20 nm to about, about 18 nm to about 22 nm, about 20 nm to about 24 nm, about 22 nm to about 26 nm, about 24 nm to about 28 nm, about 26 nm to about 30 nm, about 2 nm to about 10 nm, about 4 nm to about 12 nm, about 6 nm to about 14 nm, about 8 nm to about 16 nm, about 10 nm to about 18 nm, about 12 nm to about 20 nm, about 14 nm to about 22 nm, about 16 nm to about 24 nm, about 18 nm to about 26 nm, about 20 nm to about 28 nm, about 22 nm to about 30 nm, about 2 nm to about 12 nm, about 4 nm to about 14 nm, about 6 nm to about 16 nm, about 8 nm to about 18 nm, about 10 nm to about 20 nm, about 12 nm to about 22 nm, about 14 nm to about 24 nm, about 16 nm to about 26 nm, about 18 nm to about 28 nm, about 20 nm to about 30 nm, about 2 nm to about 5 nm, about 5 nm to about 10 nm, about 10 nm to about 15 nm, about 15 nm to about 20 nm, about 20 nm to about 25 nm, or about 25 nm to about 30 nm.

### Extracellular Vesicles

Extracellular vesicles (EVs) typically have a diameter or 20 nm to 1000 nm. Exosomes, which are small extracellular vesicles, typically are about 50-200 nm (e.g., 100-200 nm) in diameter. EVs, e.g., exosomes, are composed of a limiting lipid bilayer and a diverse set of proteins and nucleic acids (Maas, S. L. N., et al., Trends. Cell Biol. 27 (3): 172-188 (2017)). EVs, e.g., exosomes, exhibit preferential uptake in discrete cell types and tissues, and their tropism can be directed by adding proteins to their surface that interact with receptors on the surface of target cells (Alvarez-Erviti, L., et al., Nat. Biotechnol. 29 (4): 341-345 (2011)).

The EVs (e.g., exosomes) of the present disclosure can have a diameter between about 20 and about 300 nm. In certain aspects, an EV (e.g., exosome) of the present disclosure has a diameter between about 20 to about 290 nm, about 20 to about 280 nm, about 20 to about 270 nm, about 20 to about 260 nm, about 20 to about 250 nm, about 20 to about 240 nm, about 20 to about 230 nm, about 20 to about 220 nm, about 20 to about 210 nm, about 20 to about 200 nm, about 20 to about 190 nm, about 20 to about 180 nm, about 20 to about 170 nm, about 20 to about 160 nm, about 20 to about 150 nm, about 20 to about 140 nm, about 20 to about 130 nm, about 20 to about 120 nm, about 20 to about 110 nm, about 20 to about 100 nm, about 20 to about 90 nm, about 20 to about 80 nm, about 20 to about 70 nm, about 20 to about 60 nm, about 20 to about 50 nm, about 20 to about 40 nm, or about 20 to about 30 nm. The size of the EV (e.g., exosome) described herein can be measured according to methods known in the art.

Unlike antibodies, EVs (e.g., exosomes) can accommodate large numbers of molecules attached to their surface, on the order of thousands to tens of thousands of molecules per EV (e.g., exosome). EV (e.g., exosome)-drug conjugates thus represent a platform to deliver a high concentration of therapeutic compounds to discrete cell types, while at the same time limiting overall systemic exposure to the compound, which in turn, reduces off-target toxicity. The accommodation of larger numbers of molecules on the surface of EVs (e.g., exosomes) can be influenced, for example, by the type of biologically active molecule used (e.g., an antibody is bulkier than an antisense oligonucleotide), the type of membrane anchor used (e.g., a protein anchor is bulkier than a lipid or lipid plus spacer anchor), and the combinations of linkers and spacers connecting the biologically active molecule and the membrane anchor. In this respect, the present disclosure provides specific combinations of linkers and spacers connecting a biologically active molecule (e.g., an ASO) and a membrane anchor (e.g., a lipid), wherein the membrane anchor attaches the biologically active molecule to the surface of an EV (e.g., exosome).

In some aspects, the present disclosure provides a “biologically active molecule” (BAM), e.g., an ASO, attached (e.g., covalently bonded) to one or more anchoring moieties (AM) either directly or indirectly, e.g., via one or more linker combinations. The anchoring moiety can insert into the lipid bilayer of an EV, e.g., an exosome, allowing the loading of the exosome with a BAM, e.g., an ASO. Currently, a predominant obstacle to the commercialization of exosomes as a delivery vehicle for polar BAMs, e.g., ASOs, is highly inefficient loading. As shown herein, this obstacle can be overcome by using specific linkers/spacer combinations (i.e., “linkers”) connecting the BAM to the AM prior to loading them into EVs, e.g., exosomes. Thus, as described herein, the use of optimized linkers facilitates the loading of BAMs, e.g., ASOs, onto EVs, e.g., exosomes.

The composition and methods of loading EVs (e.g., exosomes) with constructs comprising BAMs, e.g., ASOs, connected to AMs (e.g., lipids, such as sterols) via optimized linkers set forth herein improve loading efficiency and BAM density as compared to the loading efficiency and BAM density previously reported for introducing unmodified BAMs into EVs (e.g., exosomes) by, for example, electroporation or cationic lipid transfection. The compositions and methods disclosed herein also significantly improve the potency of EVs (e.g., exosomes) compared to the potencies previously reported when unmodified BAMs are introduced into EVs (e.g., exosomes) by, for example, electroporation or cationic lipid transfection.

EVs (e.g., exosomes) of the present disclosure comprise a bi-lipid membrane (“exosome membrane” or “EV membrane”), comprising an interior surface (luminal surface) and an exterior surface. The interior surface faces the inner core of the EV (e.g., exosome), i.e., the lumen of the EV. The EV or exosome membrane comprises lipids and fatty acids. Exemplary lipids comprise phospholipids, glycolipids, fatty acids, sphingolipids, phosphoglycerides, sterols, cholesterols, and phosphatidylserines. The EV or exosome membrane comprises an inner leaflet and an outer leaflet. The composition of the inner and outer leaflet can be determined by transbilayer distribution assays known in the art, see, e.g., Kuypers et al., Biohim Biophys Acta 1985 819:170.

In some aspects, the composition of the outer leaflet is between about 70% and about 90% choline phospholipids, between about 0% and about 15% acidic phospholipids, and between about 5% and about 30% phosphatidylethanolamine. In some aspects, the composition of the inner leaflet is between about 15% and about 40% choline phospholipids, between about 10% and about 50% acidic phospholipids, and between about 30% and about 60% phosphatidylethanolamine. In some aspects, the EV or exosome membrane comprises one or more polysaccharides, such as glycan. Glycans on the surface of the EV or exosomes can serve as an attachment to a maleimide moiety or a linker that connect the glycan and a maleimide moiety. The glycan can be present on one or more proteins on the surface of an EV (e.g., exosome), for example, a Scaffold X, such as a PTGFRN polypeptide, or on the lipid membrane of the EV (e.g., exosome). Glycans can be modified to have thiofucose that can serve as a functional group for attaching a maleimide moiety to the glycans. In some aspects, the Scaffold X can be modified to express a high number of glycan to allow additional attachments on the EV (e.g., exosome).

### Anchoring Moiety

In some aspects, the anchoring moiety AM of Formula I of II comprises a sterol, a lipid (e.g., a phospholipid), a vitamin, a peptide, or a combination thereof and optionally spacer. In general, the AM can comprise any hydrophobic moiety or combination thereof capable of inserting into the lipid bilayer of the EV (e.g., exosome), interacting electrostatically with the surface of the EV (e.g., exosome), or a combination thereof. Suitable anchoring moieties AM capable of anchoring a biologically active molecule BAM to the surface of an EV, e.g., an exosome, comprise for example sterols (e.g., cholesterol), lipids, phospholipids, lysophospholipids, fatty acids, a vitamin (e.g., fat-soluble vitamins, scaffolding moieties (e.g., Protein X), or combinations thereof as described herein.

In some aspects, the anchoring moiety AM comprises a sterol, steroid, hopanoid, hydroxysteroid, secosteroid, or analog thereof with lipophilic properties. In some aspects, the anchoring moiety comprises a sterol, such as a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some aspects, the sterol is selected from ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, stigmasterol, or combinations thereof. Sterols can be found either as free sterols, acylated (sterol esters), alkylated (steryl alkyl ethers), sulfated (sterol sulfate), or linked to a glycoside moiety (steryl glycosides), which can be itself acylated (acylated sterol glycosides).

Sterols can be attached (via solid phase synthesis or conjugation), e.g., to a spacer SP via the available —OH group of the sterol. Exemplary sterols have the general skeleton shown below:

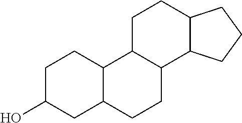 [SMILES: OC1CCC2C(CCC3C4CCCC4CCC23)C1]

For example, ergosterol has the structure below:

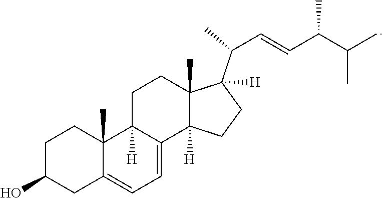

In another example, cholesterol has the structure below:

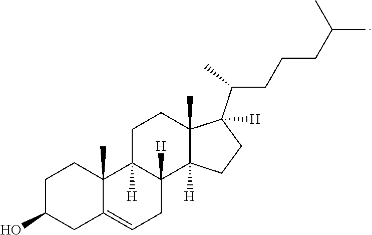

In some aspects, the anchoring moiety AM comprises, consists, or consists essentially of a sterol, cholesterol, thiocholesterol, ergosterol, 7-dehydrocholesterol, 24S-hydroxycholesterol, lanosterol, cycloartenol, fucosterol, saringosterol, campesterol, β-sitosterol, sitostanol, coprostanol, avenasterol, stigmasterol, or combinations thereof. In some specific aspects, the anchoring moiety AM comprises cholesterol.

In some aspects, the anchoring moiety AM comprises a steroid. In some aspects, the steroid is selected from dihydrotestosterone, uvaol, hecigenin, diosgenin, progesterone, or cortisol.

In some aspects, the anchoring moiety AM comprises or consists of a lipid. A lipid anchoring moiety AM can comprise any lipid known in the art, e.g., palmitic acid or glycosylphosphatidylinositols. In some aspects, the AM comprises a lipid comprising a fatty acid or a phospholipid. In some aspects, the lipid is a fatty acid, phosphatide, phospholipid (e.g., phosphatidyl choline, phosphatidyl serine, or phosphatidyl ethanolamine), or analogue thereof (e.g., phosphatidylcholine, lecithin, phosphatidylethanolamine, cephalin, or phosphatidylserine or analogue or portion thereof, such as a partially hydrolyzed portion thereof).

In some aspects, the anchoring moiety AM comprises, consists, or consists essentially of a fatty acid, e.g., a straight chain fatty acid. In some aspects, the fatty acid is a straight chain fatty acid, a branched fatty acid, a saturated fatty acid, an unsaturated fatty acid, a hydroxyl fatty acid, a polycarboxylic acid, or any combination thereof. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as an ω-3 (omega-3) or ω-6 (omega-6) fatty acid. In some aspects, the anchoring moiety AM comprises, consists, or consists essentially of a straight chain fatty, a branched fatty acid, an unsaturated fatty acid, a monounsaturated fatty acid, a polyunsaturated fatty acid, a hydroxyl fatty acid, a polycarboxylic acid, or any combination thereof.

In some aspects, the lipid, e.g., fatty acid, has a C₂-C₁₈ chain. In some aspects, the lipid, e.g., fatty acid, has a C₂, C₃, C₄, C₅, C₆, C₇, C₈, C₉, C₁₀, C₁₁, C₁₂, C₁₃, C₁₄, C₁₅, C₁₆, C₁₇, or C₁₈ chain. In some aspects, the fatty acid, has a C₂ chain. In some aspects, the fatty acid, has a C₃ chain. In some aspects, the fatty acid, has a C₄ chain. In some aspects, the fatty acid, has a C₅ chain. In some aspects, the fatty acid, has a C₆ chain. In some aspects, the fatty acid, has a C₇ chain. In some aspects, the fatty acid, has a C₈ chain. In some aspects, the fatty acid, has a C₉ chain. In some aspects, the fatty acid, has a C₁₀ chain. In some aspects, the fatty acid, has a C₁₁ chain. In some aspects, the fatty acid, has a C₁₂ chain. In some aspects, the fatty acid, has a C₁₃ chain. In some aspects, the fatty acid, has a C₁₄ chain. In some aspects, the fatty acid, has a C₁₅ chain. In some aspects, the fatty acid, has a C₁₆ chain. In some aspects, the fatty acid, has a C₁₇ chain. In some aspects, the fatty acid, has a C₁₈ chain.

In some aspects, the fatty acid, has a C₄-C₁₈ chain. In some aspects, the fatty acid, has a C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, C₂-C₁₅, C₂-C₁₆, C₂-C₁₇, C₂-C₁₈, C₃-C₄, C₃-C₅, C₃-C₆, C₃-C₇, C₃-C₈, C₃-C₉, C₃-C₁₀, C₃-C₁₁, C₃-C₁₂, C₃-C₁₃, C₃-C₁₄, C₃-C₁₅, C₃-C₁₆, C₃-C₁₇, C₃-C₁₈, C₄-C₅, C₄-C₆, C₄-C₇, C₄- C₈, C₄-C₉, C₄-C₁₀, C₄-C₁₁, C₄-C₁₂, C₄-C₁₃, C₄-C₁₄, C₄-C₁₅, C₄-C₁₆, C₄-C₁₇, C₄-C₁₈, C₅-C₆, C₅-C₇, C₅- C₈, C₅-C₉, C₅-C₁₀, C₅-C₁₁, C₅-C₁₂, C₅-C₁₃, C₅-C₁₄, C₅-C₁₅, C₅-C₁₆, C₅-C₁₇, C₅-C₁₈, C₆-C₇, C₆-C₈, C₆- C₉, C₆-C₁₀, C₆-C₁₁, C₆-C₁₂, C₆-C₁₃, C₆-C₁₄, C₆-C₁₅, C₆-C₁₆, C₆-C₁₇, C₆-C₁₈, C₇-C₈, C₇-C₉, C₇-C₁₀, C₇- C₁₁, C₇-C₁₂, C₇-C₁₃, C₇-C₁₄, C₇-C₁₅, C₇-C₁₆, C₇-C₁₇, C₇-C₁₈, C₈-C₉, C₈-C₁₀, C₈-C₁₁, C₈-C₁₂, C₈-C₁₃, C₈-C₁₄, C₈-C₁₅, C₈-C₁₆, C₈-C₁₇, C₈-C₁₈, C₉-C₁₀, C₉-C₁₁, C₉-C₁₂, C₉-C₁₃, C₉-C₁₄, C₉-C₁₅, C₉-C₁₆, C₉-C₁₇, C₉-C₁₈, C₁₀-C₁₁, C₁₀-C₁₂, C₁₀-C₁₃, C₁₀-C₁₄, C₁₀-C₁₅, C₁₀-C₁₆, C₁₀-C₁₇, C₁₀-C₁₈, C₁₁-C₁₂, C₁₁-C₁₃, C₁₁-C₁₄, C₁₁-C₁₅, C₁₁-C₁₆, C₁₁-C₁₇, C₁₁-C₁₈, C₁₂-C₁₃, C₁₂-C₁₄, C₁₂-C₁₅, C₁₂-C₁₆, C₁₂-C₁₇, C₁₂-C₁₈, C₁₃-C₁₄, C₁₃-C₁₅, C₁₃-C₁₆, C₁₃-C₁₇, C₁₃-C₁₈, C₁₄-C₁₅, C₁₄-C₁₆, C₁₄-C₁₇, C₁₄-C₁₈, C₁₅-C₁₆, C₁₅-C₁₇, C₁₅-C₁₈, C₁₆-C₁₇, C₁₆-C₁₈, or C₁₇-C₁₈ chain.

In some aspects, the anchoring moiety AM comprises two fatty acids, each of which is independently selected from a fatty acid having a chain with any one of the foregoing ranges or numbers of carbon atoms. In some aspects, one of the fatty acids is independently a fatty acid with a C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, C₂-C₁₅, C₂-C₁₆, C₂-C₁₇, C₂-C₁₈, C₃-C₄, C₃-C₅, C₃-C₆, C₃-C₇, C₃-C₈, C₃-C₉, C₃-C₁₀, C₃-C₁₁, C₃-C₁₂, C₃-C₁₃, C₃-C₁₄, C₃-C₁₅, C₃-C₁₆, C₃-C₁₇, C₃-C₁₈, C₄-C₅, C₄-C₆, C₄-C₇, C₄-C₈, C₄-C₉, C₄-C₁₀, C₄-C₁₁, C₄-C₁₂, C₄-C₁₃, C₄-C₁₄, C₄-C₁₅, C₄-C₁₆, C₄-C₁₇, C₄-C₁₈, C₅-C₆, C₅-C₇, C₅-C₈, C₅-C₉, C₅-C₁₀, C₅-C₁₁, C₅-C₁₂, C₅-C₁₃, C₅-C₁₄, C₅-C₁₅, C₅-C₁₆, C₅-C₁₇, C₅-C₁₈, C₆-C₇, C₆-C₉, C₆-C₉, C₆-C₁₀, C₆-C₁₁, C₆-C₁₂, C₆-C₁₃, C₆-C₁₄, C₆-C₁₅, C₆-C₁₆, C₆-C₁₇, C₆-C₁₈, C₇-C₈, C₇-C₉, C₇-C₁₀, C₇- C₁₁, C₇-C₁₂, C₇-C₁₃, C₇-C₁₄, C₇-C₁₅, C₇-C₁₆, C₇-C₁₇, C₇-C₁₈, C₈-C₉, C₈-C₁₀, C₈-C₁₁, C₈-C₁₂, C₈-C₁₃, C₈-C₁₄, C₈-C₁₅, C₈-C₁₆, C₈-C₁₇, C₈-C₁₈, C₉-C₁₀, C₉-C₁₁, C₉-C₁₂, C₉-C₁₃, C₉-C₁₄, C₉-C₁₅, C₉-C₁₆, C₉-C₁₇, C₉-C₁₈, C₁₀-C₁₁, C₁₀-C₁₂, C₁₀-C₁₃, C₁₀-C₁₄, C₁₀-C₁₅, C₁₀-C₁₆, C₁₀-C₁₇, C₁₀-C₁₈, C₁₁-C₁₂, C₁₁-C₁₃, C₁₁-C₁₄, C₁₁-C₁₅, C₁₁-C₁₆, C₁₁-C₁₇, C₁₁-C₁₈, C₁₂-C₁₃, C₁₂-C₁₄, C₁₂-C₁₅, C₁₂-C₁₆, C₁₂-C₁₇, C₁₂-C₁₈, C₁₃-C₁₄, C₁₃-C₁₅, C₁₃-C₁₆, C₁₃-C₁₇, C₁₃-C₁₈, C₁₄-C₁₅, C₁₄-C₁₆, C₁₄-C₁₇, C₁₄-C₁₈, C₁₅-C₁₆, C₁₅-C₁₇, C₁₅-C₁₈, C₁₆-C₁₇, C₁₆-C₁₈, or C₁₇-C₁₈ chain and the other one is independently a fatty acid with a C₂-C₃, C₂-C₄, C₂-C₅, C₂-C₆, C₂-C₇, C₂-C₈, C₂-C₉, C₂-C₁₀, C₂-C₁₁, C₂-C₁₂, C₂-C₁₃, C₂-C₁₄, C₂-C₁₅, C₂-C₁₆, C₂-C₁₇, C₂-C₁₈, C₃-C₄, C₃-C₅, C₃-C₆, C₃-C₇, C₃-C₈, C₃-C₉, C₃-C₁₀, C₃-C₁₁, C₃-C₁₂, C₃-C₁₃, C₃-C₁₄, C₃-C₁₅, C₃-C₁₆, C₃-C₁₇, C₃-C₁₈, C₄-C₅, C₄-C₆, C₄-C₇, C₄-C₈, C₄-C₉, C₄-C₁₀, C₄-C₁₁, C₄-C₁₂, C₄-C₁₃, C₄-C₁₄, C₄-C₁₅, C₄-C₁₆, C₄-C₁₇, C₄-C₁₈, C₅-C₆, C₅-C₇, C₅-C₈, C₅-C₉, C₅-C₁₀, C₅- C₁₁, C₅-C₁₂, C₅-C₁₃, C₅-C₁₄, C₅-C₁₅, C₅-C₁₆, C₅-C₁₇, C₅-C₁₈, C₆-C₇, C₆-C₈, C₆-C₉, C₆-C₁₀, C₆-C₁₁, C₆- C₁₂, C₆-C₁₃, C₆-C₁₄, C₆-C₁₅, C₆-C₁₆, C₆-C₁₇, C₆-C₁₈, C₇-C₈, C₇-C₉, C₇-C₁₀, C₇-C₁₁, C₇-C₁₂, C₇-C₁₃, C₇- C₁₄, C₇-C₁₅, C₇-C₁₆, C₇-C₁₇, C₇-C₁₈, C₈-C₉, C₈-C₁₀, C₈-C₁₁, C₈-C₁₂, C₈-C₁₃, C₈-C₁₄, C₈-C₁₅, C₈-C₁₆, C₈-C₁₇, C₈-C₁₈, C₉-C₁₀, C₉-C₁₁, C₉-C₁₂, C₉-C₁₃, C₉-C₁₄, C₉-C₁₅, C₉-C₁₆, C₉-C₁₇, C₉-C₁₈, C₁₀-C₁₁, C₁₀-C₁₂, C₁₀-C₁₃, C₁₀-C₁₄, C₁₀-C₁₅, C₁₀-C₁₆, C₁₀-C₁₇, C₁₀-C₁₈, C₁₁-C₁₂, C₁₁-C₁₃, C₁₁-C₁₄, C₁₁-C₁₅, C₁₁-C₁₆, C₁₁-C₁₇, C₁₁-C₁₈, C₁₂-C₁₃, C₁₂-C₁₄, C₁₂-C₁₅, C₁₂-C₁₆, C₁₂-C₁₇, C₁₂-C₁₈, C₁₃-C₁₄, C₁₃-C₁₅, C₁₃-C₁₆, C₁₃-C₁₇, C₁₃-C₁₈, C₁₄-C₁₅, C₁₄-C₁₆, C₁₄-C₁₇, C₁₄-C₁₈, C₁₅-C₁₆, C₁₅-C₁₇, C₁₅-C₁₈, C₁₆-C₁₇, C₁₆-C₁₈, or C₁₇-C₁₈ chain. In some aspects, each fatty acid independently has a chain of 2, 3, 4, 5, 6, 7, 8, 9, 10, 11, 12, 13, 14, 15, 16, 17 or 18 carbon atoms.

Examples of useful saturated straight-chain fatty acids include those having an even number of carbon atoms, such as butyric acid (C₄), caproic acid (C₆), caprylic acid (C₈), capric acid (C10), lauric acid (C12), myristic acid (C14), palmitic acid (C16), or stearic acid (C18), and those having an odd number of carbon atoms, such as propionic acid (C₃), n-valeric acid (C₅), enanthic acid (C₇), pelargonic acid (C₉), hendecanoic acid (C₁₁), tridecanoic acid (C₁₃), pentadecanoic acid (C₁₅), or heptadecanoic acid (C₁₇).

Examples of suitable saturated branched fatty acids include isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, or isostearic acid. Suitable saturated odd-carbon branched fatty acids include anteiso fatty acids terminating with an isobutyl group, such as 6-methyl-octanoic acid, 8-methyl-decanoic acid, 10-methyl-dodecanoic acid, 12-methyl-tetradecanoic acid, or 14-methyl-hexadecanoic acid.

Examples of suitable unsaturated fatty acids include 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, and the like.

Examples of suitable hydroxy fatty acids include a-hydroxylauric acid, a-hydroxymyristic acid, α-hydroxypalmitic acid, α-hydroxystearic acid, ω-hydroxylauric acid, a-hydroxyarachic acid, 9-hydroxy-12-octadecenoic acid, ricinoleic acid, 9-hydroxy-trans-10,12-octadecadienic acid, 9,10-dihydroxystearic acid, 12-hydroxystearic acid and the like.

Examples of suitable polycarboxylic acids include oxalic acid, malonic acid, succinic acid, glutaric acid, adipic acid, pimelic acid, suberic acid, azelaic acid, sebacic acid, D,L-malic acid, and the like.

In some aspects, each fatty acid is independently selected from propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, or stearic acid.

In some aspects, each fatty acid is independently selected from a-linolenic acid, stearidonic acid, eicosapentaenoic acid, docosahexaenoic acid, linoleic acid, gamma-linoleic acid, dihomo-gamma-linoleic acid, arachidonic acid, docosatetraenoic acid, palmitoleic acid, vaccenic acid, paullinic acid, oleic acid, elaidic acid, bosseopentaenoic acid, sardine acid, or another monounsaturated or polyunsaturated fatty acid.

In some aspects, one or both of the fatty acids is an essential fatty acid. In view of the beneficial health effects of certain essential fatty acids, the therapeutic benefits of disclosed therapeutic-loaded exosomes can be increased by including such fatty acids in the therapeutic agent. In some aspects, the essential fatty acid is an n-6 or n-3 essential fatty acid selected from the group consisting of linolenic acid, gamma-linolenic acid, dihomo-gamma-linolenic acid, arachidonic acid, adrenic acid, docosapentaenoic n-6 acid, alpha-linolenic acid, or stearidonic acid.

Fatty acid chains differ greatly in the length of their chains and can be categorized according to chain length, e.g., as short to very long. Short-chain fatty acids (SCFA) are fatty acids with chains of about five or less carbons (e.g., butyric acid). In some aspects, the fatty acid is a SCFA. Medium-chain fatty acids (MCFA) include fatty acids with chains of about 6-12 carbons, which can form medium-chain triglycerides. In some aspects, the fatty acid is a MCFA. Long-chain fatty acids (LCFA) include fatty acids with chains of 13-21 carbons. In some aspects, the fatty acid is a LCFA. In some aspects, the fatty acid is a LCFA.

In some aspects, the anchoring moiety AM is formed from a straight chain fatty that comprises, consists, or consists essentially of butyric acid, caproic acid, caprylic acid, capric acid, lauric acid, myristic acid, palmitic acid, stearic acid, or a combination thereof. In some specific aspects, the anchoring moiety AM is formed from palmitic acid.

In some aspects, the anchoring moiety AM comprises, consists, or consists essentially of a phospholipid. The structure of the phospholipid molecule generally consists of two hydrophobic fatty acid “tails” and a hydrophilic “head” consisting of a phosphate group. For example, a phospholipid can be a lipid according to the following formula:

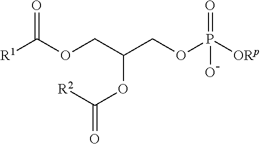

in which R^{p} is a phospholipid moiety and R¹ and R² are the same or different and each is a fatty acid moiety with or without unsaturation. A fatty acid moiety can be selected, for example, from the non-limiting group consisting of lauric acid, myristic acid, myristoleic acid, palmitic acid, palmitoleic acid, stearic acid, oleic acid, or linoleic acid.

A phospholipid moiety can be, for example, a lecithin, a phosphatidyl choline (e.g., 2 lysophosphatidyl choline), a phosphoinositol, a phosphosphingolipid, a phosphoethanolamine, phosphatidyl ethanolamine, phosphatidyl glycerol, phosphatidyl serine, phosphatidic acid, and a sphingomyelin, or any combination thereof.

The phospholipids used as anchoring moieties AM in the present disclosure can be natural or non-natural phospholipids. Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Examples of phospholipids that can be used in the anchoring moieties disclosed herein include phosphatidylethanolamines (e.g., dilauroylphosphatidyl ethanolamine, dimyristoylphosphatidyl ethanolamine, dipalmitoylphosphatidyl ethanolamine, distearoylphosphatidyl ethanolamine, dioleoylphosphatidyl ethanolamine, 1-palmitoyl-2-oleylphosphatidyl ethanolamine, 1-oleyl-2-palmitoylphosphatidyl ethanolamine, and dierucoylphosphatidyl ethanolamine), phosphatidyl glycerols (e.g., dilauroylphosphatidyl glycerol, dimyristoylphosphatidyl glycerol, dipalmitoylphosphatidyl glycerol, distearoylphosphatidyl glycerol, dioleoylphosphatidyl glycerol, 1-palmitoyl-2-oleyl-phosphatidyl glycerol, 1-oleyl-2-palmitoyl-phosphatidyl glycerol, and dierucoylphosphatidyl glycerol); phosphatidyl serines (e.g., such as dilauroylphosphatidyl serine, dimyristoylphosphatidyl serine, dipalmitoylphosphatidyl serine, distearoylphosphatidyl serine, dioleoylphosphatidyl serine, 1-palmitoyl-2-oleyl-phosphatidyl serine, 1-oleyl-2-palmitoyl-phosphatidyl serine, and dierucoylphosphatidyl serine); phosphatidic acids (e.g., dilauroylphosphatidic acid, dimyristoylphosphatidic acid, dipalmitoylphosphatidic acid, distearoylphosphatidic acid, dioleoylphosphatidic acid, 1-palmitoyl-2-oleylphosphatidic acid, 1-oleyl-2-palmitoyl-phosphatidic acid, and dierucoylphosphatidic acid); and phosphatidyl inositols (e.g., dilauroylphosphatidyl inositol, dimyristoylphosphatidyl inositol, dipalmitoylphosphatidyl inositol, distearoylphosphatidyl inositol, dioleoylphosphatidyl inositol, 1-palmitoyl-2-oleyl-phosphatidyl inositol, 1-oleyl-2-palmitoyl-phosphatidyl inositol, and dierucoylphosphatidyl inositol. In some aspects, the AM is formed from dipalmitoylphosphatidic acid.

Phospholipids can be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some aspects, the anchoring moiety AM comprises a phospholipid, e.g., a symmetric phospholipid, with 10 carbons (C10), twelve carbons (C12), fourteen carbons (C14), sixteen carbons (C16) or eighteen carbons (C18). In some aspects, the anchoring moiety AM comprises a symmetric phospholipid with fourteen carbons (C14). In some aspects, the anchoring moiety AM comprises a symmetric phospholipid with sixteen carbons (C16). In some aspects, the anchoring moiety AM comprises a symmetric phospholipid with eighteen carbons (C18). In some aspects, the phospholipid in phosphatidyl ethanolamine (PE). Accordingly, in some aspects, the anchoring moiety AM comprises a C14 PE. In some aspects, the anchoring moiety AM comprises a C16 PE. In some aspects, the anchoring moiety AM comprises a C18 PE. In some aspects, the acyl chains of the PE contain no insaturations. Accordingly, in some aspects, the anchoring moiety AM comprises a C14:0 PE, a C16:0 PE, or a C18:0 PE. In some aspects, the anchoring moiety AM comprises 16:0 1,2-dipalmitoyl-sn-glycero-3-phosphoethanolamine-N-[3-(2-pyridyldithio) propionate] (16:0 PDP PE).

In some aspects, the anchoring moiety AM comprises a phospholipid comprising a cyanuric acid (cyanur) group. In some aspects, the phospholipid comprising a cyanuric acid group is a PE. In some aspects, the phospholipid comprising a cyanuric acid group is a C14:0 PE, a C16:0 PE, or a C18:0 PE. In some aspects, the anchoring moiety AM comprises 16:0 1,2-dipalmitoyl-sn-glycero-3-phosphoethanolamine-N-[4-(p-maleimidomethyl)cyclohexane-carboxamide] (16:0 PE MCC). In some aspects, the anchoring moiety AM comprises 16:0 1,2-dipalmitoyl-sn-glycero-3-phosphoethanolamine-N-(cyanur)(16:0 Cyanur PE).

In some aspects, the anchoring moiety AM comprises at least one symmetric phospholipid. Symmetric phospholipids can be selected from the non-limiting group consisting of 1,2 dipropionyl sn-glycero 3 phosphocholine (03:0 PC), 1,2 dibutyryl sn glycero 3 phosphocholine (04:0 PC), 1,2 dipentanoyl sn glycero 3 phosphocholine (05:0 PC), 1,2 dihexanoyl sn glycero 3 phosphocholine (06:0 PC), 1,2 diheptanoyl sn glycero 3 phosphocholine (07:0 PC), 1,2 dioctanoyl sn glycero 3 phosphocholine (08:0 PC), 1,2 dinonanoyl sn glycero 3 phosphocholine (09:0 PC), 1,2 didecanoyl sn glycero 3 phosphocholine (10:0 PC), 1,2 diundecanoyl sn glycero 3 phosphocholine (11:0 PC, DUPC), 1,2 dilauroyl sn glycero 3 phosphocholine (12:0 PC), 1,2 ditridecanoyl sn glycero 3 phosphocholine (13:0 PC), 1,2 dimyristoyl sn glycero 3 phosphocholine (14:0 PC, DMPC), 1,2 dipentadecanoyl sn glycero 3 phosphocholine (15:0 PC), 1,2 dipalmitoyl sn glycero 3 phosphocholine (16:0 PC, DPPC), 1,2 diphytanoyl sn glycero 3 phosphocholine (4ME 16:0 PC), 1,2 diheptadecanoyl sn glycero 3 phosphocholine (17:0 PC), 1,2 distearoyl sn glycero 3 phosphocholine (18:0 PC, DSPC), 1,2 dimyristelaidoyl sn glycero 3 phosphocholine (14:1 (49-Trans) PC), 1,2 dipalmitoleoyl sn glycero 3 phosphocholine (16:1 (49-Cis) PC), 1,2 dipalmitelaidoyl sn glycero 3 phosphocholine (16:1 (49-Trans) PC), 1,2 dipetroselenoyl sn glycero 3 phosphocholine (18:1 (46-Cis) PC), 1,2 dioleoyl sn glycero 3 phosphocholine (18:1 (49-Cis) PC, DOPC), 1,2 dielaidoyl sn glycero 3 phosphocholine (18:1 (49-Trans) PC), 1,2 dilinoleoyl sn glycero 3 phosphocholine (18:2 (Cis) PC, DLPC), 1,2 dilinolenoyl sn glycero 3 phosphocholine (18:3 (Cis) PC, DLnPC), 1,2 dihexanoyl sn glycero 3 phosphoethanolamine (06:0 PE), 1,2 dioctanoyl sn glycero 3 phosphoethanolamine (08:0 PE), 1,2 didecanoyl sn glycero 3 phosphoethanolamine (10:0 PE), 1,2 dilauroyl sn glycero 3 phosphoethanolamine (12:0 PE), 1,2 dimyristoyl sn glycero 3 phosphoethanolamine (14:0 PE), 1,2 dipentadecanoyl sn glycero 3 phosphoethanolamine (15:0 PE), 1,2 dipalmitoyl sn glycero 3 phosphoethanolamine (16:0 PE), 1,2 diphytanoyl sn glycero 3 phosphoethanolamine (4ME 16:0 PE), 1,2 diheptadecanoyl sn glycero 3 phosphoethanolamine (17:0 PE), 1,2 distearoyl sn glycero 3 phosphoethanolamine (18:0 PE, DSPE), 1,2 dipalmitoleoyl sn glycero 3 phosphoethanolamine (16:1 PE), 1,2 dioleoyl sn glycero 3 phosphoethanolamine (18:1 (49-Cis) PE, DOPE), 1,2 dielaidoyl sn glycero 3 phosphoethanolamine (18:1 (49-Trans) PE), 1,2 dilinoleoyl sn glycero 3 phosphoethanolamine (18:2 PE, DLPE), 1,2 dilinolenoyl sn glycero 3 phosphoethanolamine (18:3 PE, DLnPE), 1,2 di O octadecenyl sn glycero 3 phosphocholine (18:0 Diether PC), 1,2 dioleoyl sn glycero 3 phospho rac (1 glycerol) sodium salt (DOPG), and any combination thereof.

In some aspects, the anchoring moiety AM comprises at least one symmetric phospholipid selected from the non-limiting group consisting of DLPC, DMPC, DOPC, DPPC, DSPC, DUPC, 18:0 Diether PC, DLnPC, DAPC, DHAPC, DOPE, 4ME 16:0 PE, DSPE, DLPE,DLnPE, DAPE, DHAPE, DOPG, and any combination thereof.

In some aspects, the anchoring moiety AM comprises at least one asymmetric phospholipid. Asymmetric phospholipids can be selected from the non-limiting group consisting of 1 myristoyl 2 palmitoyl sn glycero 3 phosphocholine (14:0-16:0 PC, MPPC), 1 myristoyl 2 stearoyl sn glycero 3 phosphocholine (14:0-18:0 PC, MSPC), 1 palmitoyl 2 acetyl sn glycero 3 phosphocholine (16:0-02:0 PC), 1 palmitoyl 2 myristoyl sn glycero 3 phosphocholine (16:0-14:0 PC, PMPC), 1 palmitoyl 2 stearoyl sn glycero 3 phosphocholine (16:0-18:0 PC, PSPC), 1 palmitoyl 2 oleoyl sn glycero 3 phosphocholine (16:0-18:1 PC, POPC), 1 palmitoyl 2 linoleoyl sn glycero 3 phosphocholine (16:0-18:2 PC, PLPC), 1 palmitoyl 2 arachidonoyl sn glycero 3 phosphocholine (16:0-20:4 PC), 1 palmitoyl 2 docosahexaenoyl sn glycero 3 phosphocholine (14:0-22:6 PC), 1 stearoyl 2 myristoyl sn glycero 3 phosphocholine (18:0-14:0 PC, SMPC), 1 stearoyl 2 palmitoyl sn glycero 3 phosphocholine (18:0-16:0 PC, SPPC), 1 stearoyl 2 oleoyl sn glycero 3 phosphocholine (18:0-18:1 PC, SOPC), 1 stearoyl 2 linoleoyl sn glycero 3 phosphocholine (18:0-18:2 PC), 1 stearoyl 2 arachidonoyl sn glycero 3 phosphocholine (18:0-20:4 PC), 1 stearoyl 2 docosahexaenoyl sn glycero 3 phosphocholine (18:0-22:6 PC), 1 oleoyl 2 myristoyl sn glycero 3 phosphocholine (18:1-14:0 PC, OMPC), 1 oleoyl 2 palmitoyl sn glycero 3 phosphocholine (18:1-16:0 PC, OPPC), 1 oleoyl 2 stearoyl sn glycero 3 phosphocholine (18:1-18:0 PC, OSPC), 1 palmitoyl 2 oleoyl sn glycero 3 phosphoethanolamine (16:0-18:1 PE, POPE), 1 palmitoyl 2 linoleoyl sn glycero 3 phosphoethanolamine (16:0-18:2 PE), 1 palmitoyl 2 arachidonoyl sn glycero 3 phosphoethanolamine (16:0-20:4 PE), 1 palmitoyl 2 docosahexaenoyl sn glycero 3 phosphoethanolamine (16:0-22:6 PE), 1 stearoyl 2 oleoyl sn glycero 3 phosphoethanolamine (18:0-18:1 PE), 1 stearoyl 2 linoleoyl sn glycero 3 phosphoethanolamine (18:0-18:2 PE), 1 stearoyl 2 arachidonoyl sn glycero 3 phosphoethanolamine (18:0-20:4 PE), 1 stearoyl 2 docosahexaenoyl sn glycero 3 phosphoethanolamine (18:0-22:6 PE), 1 oleoyl 2 cholesterylhemisuccinoyl sn glycero 3 phosphocholine (OChemsPC), and any combination thereof.

To provide improved nuclease resistance, cellular uptake efficiency, and a more remarkable RNA interference effect, phosphatidylethanolamines can be used as an anchoring moiety AM, for example, dimyristoylphosphatidyl ethanolamine, dipalmitoylphosphatidyl ethanolamine, 1-palmitoyl-2-oleyl-phosphatidyl ethanolamine, and dioleoylphosphatidyl ethanolamine.

In some aspects, the anchoring moiety AM comprises or consists of a lysolipid, e.g., a lysophospholipid. Lysolipids are derivatives of a lipid in which one or both fatty acyl chains have been removed, generally by hydrolysis. Lysophospholipids are derivatives of a phospholipid in which one or both fatty acyl chains have been removed by hydrolysis.

In some aspects, the anchoring moiety comprises any of the phospholipids disclosed herein, in which one or both acyl chains have been removed via hydrolysis, and therefore the resulting lysophospholipid comprises one or no fatty acid acyl chain.

In some aspects, the anchoring moiety comprises a lysoglycerophospholipid, a lysoglycosphingoliopid, a lysophosphatidylcholine, a lysophosphatidylethanolamine, a lysophosphatidylinositol, or a lysophosphatidylserine.

In some aspect, the anchoring moiety AM comprises a lysolipid selected from the non-limiting group consisting of 1 hexanoyl 2 hydroxy sn glycero 3 phosphocholine (06:0 Lyso PC), 1 heptanoyl 2 hydroxy sn glycero 3 phosphocholine (07:0 Lyso PC), 1 octanoyl 2 hydroxy sn glycero 3 phosphocholine (08:0 Lyso PC), 1 nonanoyl 2 hydroxy sn glycero 3 phosphocholine (09:0 Lyso PC), 1 decanoyl 2 hydroxy sn glycero 3 phosphocholine (10:0 Lyso PC), 1 undecanoyl 2 hydroxy sn glycero 3 phosphocholine (11:0 Lyso PC), 1 lauroyl 2 hydroxy sn glycero 3 phosphocholine (12:0 Lyso PC), 1 tridecanoyl 2 hydroxy sn glycero 3 phosphocholine (13:0 Lyso PC), 1 myristoyl 2 hydroxy sn glycero 3 phosphocholine (14:0 Lyso PC), 1 pentadecanoyl 2 hydroxy sn glycero 3 phosphocholine (15:0 Lyso PC), 1 palmitoyl 2 hydroxy sn glycero 3 phosphocholine (16:0 Lyso PC), 1 heptadecanoyl 2 hydroxy sn glycero 3 phosphocholine (17:0 Lyso PC), 1 stearoyl 2 hydroxy sn glycero 3 phosphocholine (18:0 Lyso PC), or 1 oleoyl 2 hydroxy sn glycero 3 phosphocholine (18:1 Lyso PC), 1 myristoyl 2 hydroxy sn glycero 3 phosphoethanolamine (14:0 Lyso PE), 1 palmitoyl 2 hydroxy sn glycero 3 phosphoethanolamine (16:0 Lyso PE), 1 stearoyl 2 hydroxy sn glycero 3 phosphoethanolamine (18:0 Lyso PE), 1 oleoyl 2 hydroxy sn glycero 3 phosphoethanolamine (18:1 Lyso PE), 1 hexadecyl sn glycero 3 phosphocholine (C16 Lyso PC), and any combination thereof.

In some aspects, the anchoring moiety AM comprises, consists, or consists essentially of a vitamin, e.g., a lipophilic vitamin. Suitable vitamins include, e.g., vitamin A, vitamin B (e.g., vitamin B3 (niacin), vitamin B6 (pyridoxine), vitamin B9 (folic acid), or vitamin B12 (riboflavin)), vitamin E (tocopherol or tocotrienol), vitamin D (e.g., vitamin D₂ or ergocalciferol, vitamin D₃ or cholecalciferol, or a combination thereof), vitamin K, or a combination thereof. In some apsects, the vitamin is tocopherol, tocotrienol, vitamin D, vitamin K, riboflavin, niacin, or pyridoxine. In some aspects, the vitamin is tocopherol.

In some aspects, the anchoring moiety AM comprises or consists of vitamin D. Vitamin D is a group of fat-soluble secosteroids responsible for increasing intestinal absorption of calcium, magnesium, and phosphate, and many other biological effectsIn humans, the most important compounds in this group are vitamin D₃ (also known as cholecalciferol) and vitamin D₂ (ergocalciferol).

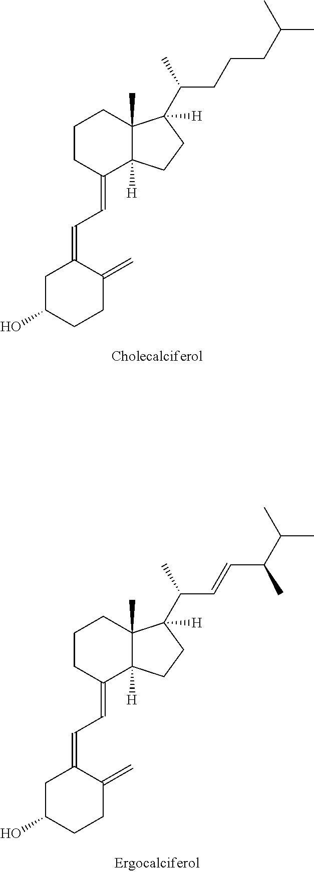 [SMILES: C=C1CC[C@H](O)C/C1=C/C=C1\CCC[C@]2(C)[C@@H]([C@H](C)CCCC(C)C)CC[C@@H]12 ; C=C1CC[C@H](O)C/C1=C/C=C1\CCC[C@]2(C)[C@@H]([C@H](C)/C=C/[C@H](C)C(C)C)CC[C@@H]12]

In some aspects, the anchoring moiety AM comprises or consists of vitamin B9 (folic acid).

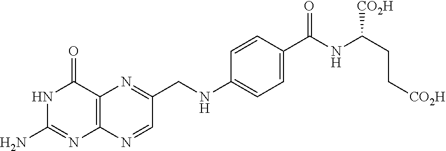 [SMILES: Nc1nc2ncc(CNc3ccc(C(=O)N[C@@H](CCC(=O)O)C(=O)O)cc3)nc2c(=O)[nH]1]

In some aspects, the anchoring moiety AM comprises or consists of vitamin B2 (riboflavin).

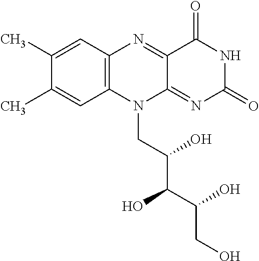 [SMILES: Cc1cc2nc3c(=O)[nH]c(=O)nc-3n(C[C@H](O)[C@H](O)[C@H](O)CO)c2cc1C]

In some aspects, the anchoring moiety AM comprises or consists of vitamin B₃ (niacin)

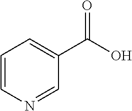

In some aspects, the anchoring moiety AM comprises or consists of vitamin B₆ (pyridoxine).

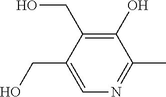

In some aspects, the anchoring moiety AM comprises or consists of vitamin A. Vitamin A is a group of unsaturated nutritional organic compounds that includes retinol, retinal, retinoic acid, and several provitamin A carotenoids (most notably beta-carotene). In some aspects, the anchoring moiety comprises retinol. In some aspects, the anchoring moiety comprises a retinoid. Retinoids are a class of chemical compounds that are vitamers of vitamin A or are chemically related to it. In some aspects, the anchoring moiety comprises a first generation retinoid (e.g., retinol, tretinoin, isotreatinoin, or alitretinoin), a second-generation retinoid (e.g., etretinate or acitretin), a third-generation retinoid (e.g., adapalene, bexarotene, or tazarotene), or any combination thereof.

First-generation retinoids

| | |
|---|---|
| retinol | 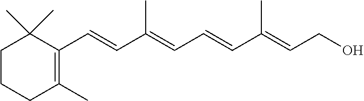 [SMILES: CC1=C(/C=C/C(C)=C/C=C/C(C)=C/CO)C(C)(C)CCC1] |
| tretinoin (all-trans-retinoic acid) | 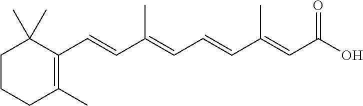 [SMILES: CC1=C(/C=C/C(C)=C/C=C/C(C)=C/C(=O)O)C(C)(C)CCC1] |
| isotreatinoin (13-cis-retinoic acid) | 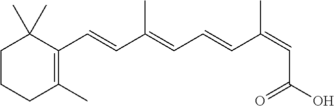 [SMILES: CC1=C(/C=C/C(C)=C/C=C/C(C)=C\C(=O)O)C(C)(C)CCC1] |
| alitretinoin (9-cis-retinoic acid) | 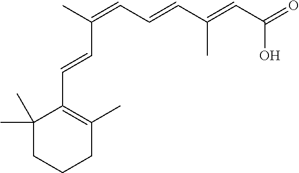 [SMILES: CC1=C(/C=C/C(C)=C\C=C\C(C)=C\C(=O)O)C(C)(C)CCC1] |

Second-generation retinoids

| | |
|---|---|
| etretinate | 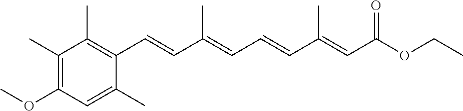 [SMILES: CCOC(=O)/C=C(C)/C=C/C=C(C)/C=C/c1c(C)cc(OC)c(C)c1C] |
| acitretin | 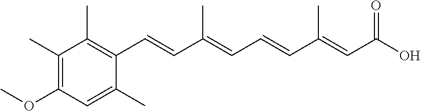 [SMILES: COc1cc(C)c(/C=C/C(C)=C/C=C/C(C)=C/C(=O)O)c(C)c1C] |

Third-generation retinoids

| | |
|---|---|
| adapalene | 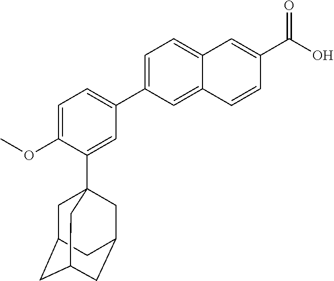 [SMILES: COc1ccc(-c2ccc3cc(C(=O)O)ccc3c2)cc1C12CC3CC(CC(C3)C1)C2] |
| bexarotene | 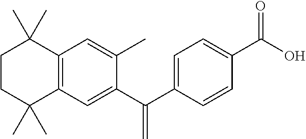 [SMILES: C=C(c1ccc(C(=O)O)cc1)c1cc2c(cc1C)C(C)(C)CCC2(C)C] |
| tazarotene | 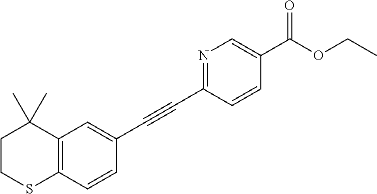 [SMILES: CCOC(=O)c1ccc(C#Cc2ccc3c(c2)C(C)(C)CCS3)nc1] |

In some aspects, the anchoring moiety AM comprises or consists of vitamin E. Tocopherols are a class of methylated phenols many of which have vitamin E activity. Thus, in some aspects, the anchoring moiety comprises alpha-tocopherol, beta-tocopherol, gamma-tocopherol, delta-tocopherol, or a combination thereof.

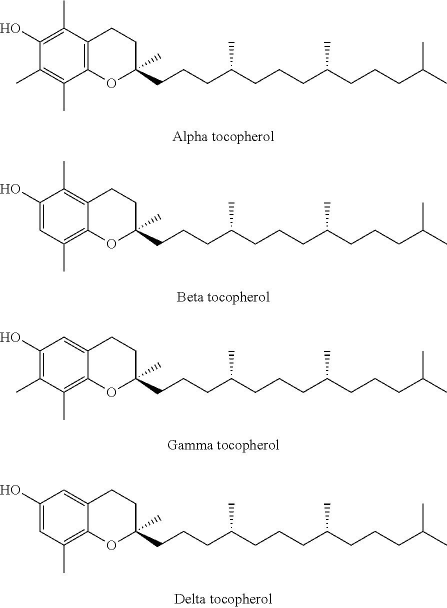 [SMILES: Cc1c(C)c2c(c(C)c1O)CC[C@@](C)(CCC[C@H](C)CCC[C@H](C)CCCC(C)C)O2 ; Cc1cc(O)c(C)c2c1O[C@](C)(CCC[C@H](C)CCC[C@H](C)CCCC(C)C)CC2 ; Cc1c(O)cc2c(c1C)O[C@](C)(CCC[C@H](C)CCC[C@H](C)CCCC(C)C)CC2 ; Cc1cc(O)cc2c1O[C@](C)(CCC[C@H](C)CCC[C@H](C)CCCC(C)C)CC2]

Tocotrienols also have vitamin E activity. The structural difference between tocotrienols and tocopherols is that tocotrienols have unsaturated isoprenoid side chain with three carbon-carbon double bonds versus saturated side chains for tocopherols. In some aspects, the anchoring moiety comprises alpha-tocotrienol, beta-tocotrienol, gamma-tocotrienol, delta-tocotrienol, or a combination thereof. Tocotrienols can be represented by the formula below

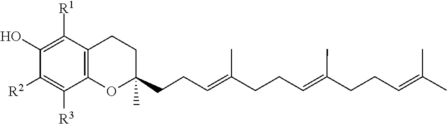

alpha (α)-Tocotrienol: R¹=Me, R²=Me, R³=Me;
beta (β)-Tocotrienol: R¹=Me, R²═H, R³=Me;
gamma (γ)-Tocotrienol: R¹=H, R²=Me, R³=Me;
delta (δ)-Tocotrienol: R¹=H, R²═H, R³=Me.

In some aspects, the anchoring moiety AM comprises or consists of vitamin K. Chemically, the vitamin K family comprises 2-methyl-1.4-naphthoquinone (3-) derivatives. Vitamin K includes two natural vitamers: vitamin K₃ and vitamin K₂. The structure of vitamin K₁ (also known as phytonadione, phylloquinone, or (E)-phytonadione) is marked by the presence of a phytyl group. The structures of vitamin K₂ (menaquinones) are marked by the polyisoprenyl side chain present in the molecule that can contain six to 13 isoprenyl units. Thus, vitamin K₂ consists of a number of related chemical subtypes, with differing lengths of carbon side chains made of isoprenoid groups of atoms. MK-4 is the most common form of vitamin K₂. Long chain forms, such as MK-7, MK-8 and MK-9 are predominant in fermented foods. Longer chain forms of vitamin K₂ such as MK-10 to MK-13 are synthesized by bacteria, but they are not well absorbed and have little biological function. In addition to the natural forms of vitamin K, there is a number of synthetic forms of vitamin K such as vitamin K₃ (menadione; 2-methylnaphthalene-1,4-dione), vitamin K₄, and vitamin K₅.

Accordingly, in some aspects, the anchoring moiety comprises vitamin K₁, K₂ (e.g., MK-4, MK-5, MK-6, MK-7, MK-8, MK-9, MK-10, MK-11, MK-12, or MK-13), K₃, K₄, K₅, or any combination thereof.

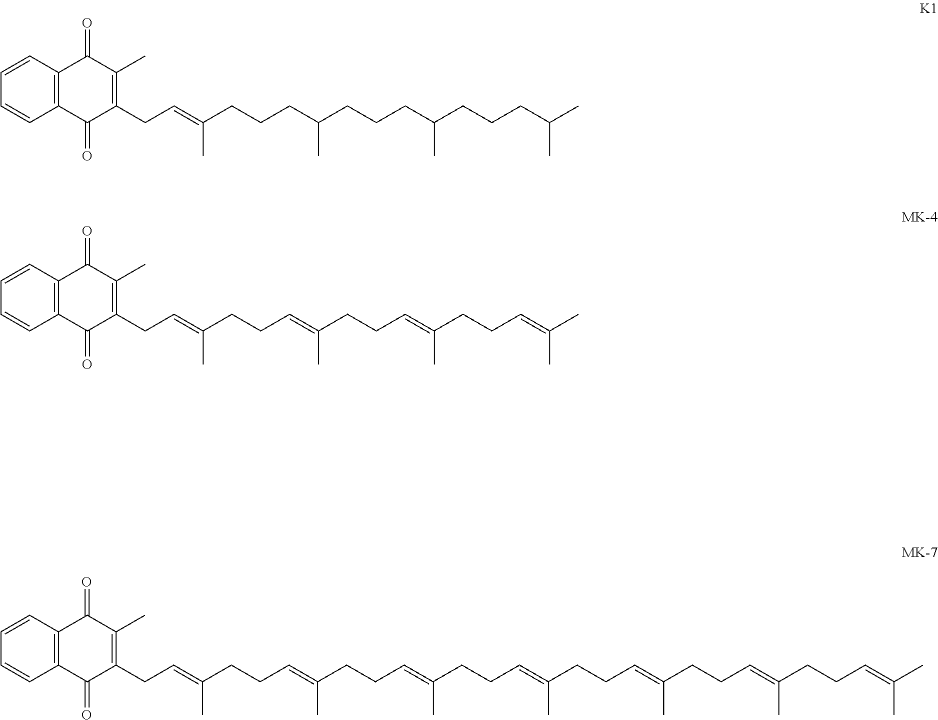 [SMILES: CC1=C(C/C=C(\C)CCCC(C)CCCC(C)CCCC(C)C)C(=O)c2ccccc2C1=O ; CC(C)=CCC/C(C)=C/CC/C(C)=C/CC/C(C)=C/CC1=C(C)C(=O)c2ccccc2C1=O ; CC(C)=CCC/C(C)=C/CC/C(C)=C/CC/C(C)=C/CC/C(C)=C/CC/C(C)=C/CC/C(C)=C/CC1=C(C)C(=O)c2ccccc2C1=O]

Chemically, the vitamin K family comprises 2-methyl-1,4-naphthoquinone (3-) derivatives. Vitamin K includes two natural vitamers: vitamin K₁ (phylloquinone) and vitamin K₂ (menaquinone). Vitamin K₂, in turn, consists of a number of related chemical subtypes, with differing lengths of carbon side chains made of isoprenoid groups of atoms. The two most studied ones are menaquinone-4 (MK-4) and menaquinone-7 (MK-7). Thus, in some aspects, the vitamin K is MK-4, MK-5, or a combination thereof.

In some aspects, the anchoring moiety AM can comprise a scaffold protein (e.g., a Scaffold X protein, such as PTGFRN or a fragment thereof), or a binding molecule which can bind to a scaffold protein present in the EV (e.g., exosome) membrane, for example, an antibody or a binding portion thereof that can specifically bind to PTGRN natively or recombinantly expressed on the surface of the EV (e.g., exosome). Thus, in some aspects, the anchoring moiety AM and/or the scaffold moiety is Scaffold X.

In some aspects, one or more scaffold moieties can be CD47, CD55, CD49, CD40, CD133, CD59, glypican-1, CD9, CD63, CD81, integrins, selectins, lectins, cadherins, other similar polypeptides known to those of skill in the art, or any combination thereof. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); or any combination thereof.

In other aspects, one or more scaffold moieties are expressed in the membrane of the EVs (e.g., exosomes) by recombinantly expressing the scaffold moieties in the producer cells. The EVs (e.g., exosomes) obtained from the producer cells can be further modified to be conjugated to a maleimide moiety or to a linker. In other aspects, the scaffold moiety, e.g., Scaffold X, is deglycosylated. In some aspects, the scaffold moiety, e.g., Scaffold X, is highly glycosylated, e.g., higher than naturally-occurring Scaffold X under the same condition.

In some aspects, the Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATPIA1, ATPIA2, ATP1A3, ATPIA4, ATPIB3, ATP2B1, ATP2B2, ATP2B3, ATP2B4 proteins); a functional fragment thereof; and any combination thereof.

In some aspects, the Scaffold X comprises Prostaglandin F2 receptor negative regulator (the PTGFRN polypeptide). The PTGFRN polypeptide can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor -associated protein, or CD315.

In some aspects, the Scaffold X is PTGFRN protein or a functional fragment thereof. In some aspects, the Scaffold X comprises an amino acid sequence as set forth in SEQ ID NO: 302. In some aspects, the Scaffold X comprises an amino acid sequence at least 50%, at least 55%, at least 60%, at least 65%, at least 70%, at least 75%, at least 80%, at least 85%, at least 90%, at least 95%, at least 96%, at least 97%, at least 98%, at least 99%, or about 100% identical to SEQ ID NO: 302.

Non-limiting examples of other Scaffold X proteins can be found at US Patent No. U.S. Pat. No. 10,195,290B1, issued Feb. 5, 2019, which is incorporated by reference in its entirety.

In the context of the present disclosure, a combination of lipid moieties, e.g., different fatty acids, different sterols, different vitamins, or combinations thereof, in an anchoring moiety AM means that some of the constructs disclosed herein, e.g., constructs of Formula I, in a population of constructs can have different anchoring moieties AM and/or linkers. For example, some constructs of Formula I will comprise a fatty acid, whereas other constructs can comprise a vitamin or a sterol. In another example, an anchoring moiety AM can comprise two lipids, e.g., a phospholipid and a fatty acid, or two phospholipids, or two fatty acids, or a lipid and a vitamin, or cholesterol and a vitamin, etc., which taken together have 6-30 carbon atoms (i.e., an equivalent carbon number (ECN) of 6-30). Selecting combinations of constructs with different anchoring moieties AM and/or linkers generally results in better packing on the lipids in the membrane on the EV (e.g., an exosome), which can result in higher loading efficiency and BAM density.

Generally, anchoring moieties are chemically attached, e.g., via solid phase synthesis. However, an anchoring moiety AM can be attached to a biologically active molecule BAM enzymatically. The anchoring moiety AM can be conjugated to a biologically active molecule BAM directly or indirectly via a linker or spacer combination, as described herein, at any chemically feasible location, e.g., at the 5′ and/or 3′ end of a nucleotide sequence, e.g., an ASO. In some aspects, the anchoring moiety AM is conjugated only to the 3′ end of the biologically active molecule BAM, directly or indirectly via a cleavable linker disclosed herein. In some aspects, the anchoring moiety AM is conjugated only to the 5′ end of a nucleotide sequence, e.g., an ASO. In one aspect, the anchoring moiety AM is conjugated at a location which is not the 3′ end or 5′ end of a nucleotide sequence, e.g., an ASO.

In some aspects, anchoring moieties AM of the present disclosure can comprise any of the hydrophobic group modifications disclosed below:

| Modification | Modfiying Group |
|---|---|
| S-Palmitoylation | 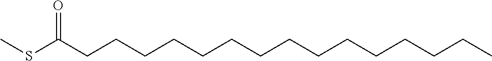 [SMILES: CCCCCCCCCCCCCCCC(=O)SC] |
| N-Palmitoylation | 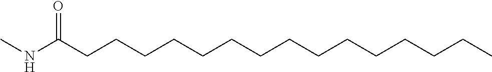 [SMILES: CCCCCCCCCCCCCCCC(=O)NC] |
| N-Myristoylation | 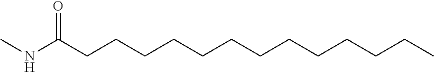 [SMILES: CCCCCCCCCCCCCC(=O)NC] |
| O-Acylation | 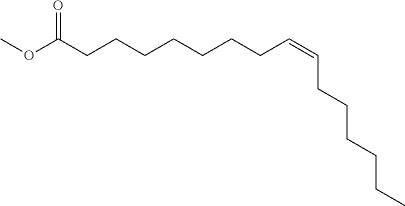 [SMILES: CCCCCC/C=C\CCCCCCCC(=O)OC] |
| Farnesylation | 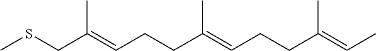 [SMILES: C/C=C(\C)CC/C=C(\C)CC/C=C(\C)CSC] |
| Geranylgeranylation | 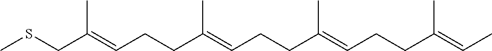 [SMILES: C/C=C(\C)CC/C=C(\C)CC/C=C(\C)CC/C=C(\C)CSC] |
| Cholesterol | 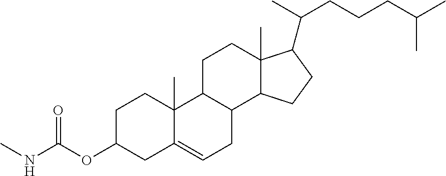 [SMILES: CNC(=O)OC1CCC2(C)C(=CCC3C2CCC2(C)C(C(C)CCCC(C)C)CCC32)C1] |

In some aspects, the anchoring moiety AM can comprise a spacer SP (e.g., SP₁ or SP₂) that enables connectivity to the BAM and includes the cleavable linkage, as described herein (e.g., L₁). Suitable spacers are described herein.

### Cleavable Linkage—Cell Penetrating Peptide

The present disclosure provides a construct of Formula I or II that comprises one or more cleavable linkages: L₁ and L₂ and L₃, in which one of the cleavable linkages comprises a cell penetrating peptide.

The term “cleavable linker” refers to a linker or spacer comprising at least one linkage or chemical bond that can be broken or cleaved under certain physiological conditions. As used herein, the term “cleave” refers to the breaking of one or more chemical bonds in a relatively large molecule in a manner that produces two or more relatively smaller molecules. Cleavage can be mediated, e.g., by a nuclease, peptidase, protease, phosphatase, oxidase, or reductase, for example, or by specific physicochemical conditions, e.g., redox environment, pH, presence of reactive oxygen species, or specific wavelengths of light.

In some aspects, two of L₁ and L₂ and L₃ are the same in Formula I or II. In some aspects, each of L₁ and L₂ and L₃ is different in Formula I or II. In some aspects, two of L₁, L₂, and L₃ are absent in Formula I or II, and the remaining cleavable linkage comprises the cell penetrating peptide. In an example, L₁ and L₂ are absent in Formula I or II, and L₃ comprises the cell penetrating peptide. In some aspects, one of L₁, L₂, and L₃ is absent from Formula I or II.

In the construct of Formulas I and II, at least one of the cleavable linkages comprises a cell penetrating peptide (CPP). As used herein, a “cell penetrating peptide” is a short peptide (e.g., 30 amino acids or fewer, such as 29 amino acids or fewer, 28 amino acids or fewer, 27 amino acids or fewer, 26 amino acids or fewer, 25 amino acids or fewer, 24 amino acids or fewer, 23 amino acids or fewer, 22 amino acids or fewer, 21 amino acids or fewer, 20 amino acids or fewer, 19 amino acids or fewer, 18 amino acids or fewer, 17 amino acids or fewer, 16 amino acids or fewer, 15 amino acids or fewer, 14 amino acids or fewer, 13 amino acids or fewer, 12 amino acids or fewer, 11 amino acids or fewer, 10 amino acids or fewer, 9 amino acids or fewer, 8 amino acids or fewer, 7 amino acids or fewer, 6 amino acids or fewer, 5 amino acids or fewer, 4 amino acids or fewer, 3 amino acids or fewer, or 2 amino acids) that can penetrate into cells to facilitate transfer of a BAM across the plasma membrane. In an aspect, the cell penetrating peptide can comprise 3 to 30 amino acid residues (e.g., 3 to 25 amino acid residues, 3 to 20 amino acid residues, 3 to 15 amino acid residues, 3 to 10 amino acid residues, 3 to 9 amino acid residues, 3 to 6 amino acid residues, 5 to 25 amino acid residues, 5 to 20 amino acid residues, 5 to 15 amino acid residues, 5 to 10 amino acid residues, or 5 to 9 amino acid residues).

In some aspects, the cell penetrating peptide can be linear or cyclic. In some aspects, the cell penetrating peptide is linear.

In some aspects, the cell penetrating peptide can be protein-derived, synthetic, or chimeric. In some aspects, the cell penetrating peptide can be classified as cationic, amphipathic (e.g., primary or secondary), non-amphipathic, hydrophilic, and/or hydrophobic. In some aspects, the cell penetrating peptide can be classified as cationic and hydrophobic. In some aspects, the cell penetrating peptide can be classified as cationic and hydrophilic.

In an aspect, the cell penetrating peptide is cationic, including polycationic. The charge on the cationic cell penetrating peptide can be +1 or more (e.g., +2 or more, +3 or more, +4 or more, +5 or more, +6 or more, +7 or more, +8 or more, +9 or more, +10 or more, +11 or more, +12 or more, +14 or more, +16 or more, +18 or more, +20 or more, +22 or more, +24 or more, +26 or more, or +28 or more). In general, the charge will be +30 or less (e.g., +28 or less, +26 or less, +24 or less, +22 or less, +20 or less, +18 or less, +16 or less, +14 or less, +12 or less, +11 or less, +10 or less, +9 or less, +8 or less, +7 or less, +6 or less, +5 or less, +4 or less, or +2 or less). In some aspects, the charge on the cell penetrating peptide will be in a range of 1 to 30, 1 to 25, 1 to 20, 1 to 15, 1 to 10, 1 to 8, 1 to 5, 2 to 20, 2 to 15, 2 to 10, 2 to 8, 2 to 5, 3 to 30, 3 to 25, 3 to 20, 3 to 15, 3 to 10, 3 to 8, 3 to 5, 4 to 30, 4 to 25, 4 to 15, 4 to 10, 4 to 8, 5 to 30, 5 to 25, 5 to 20, 5 to 15, 5 to 10, or 5 to 8. In an aspect, a cationic cell penetrating peptide can comprise one or more (e.g., 1, 2, 3, 4, 5, 6, 7, 8, 9, 10, 11, 12, 13, 14, or 15) residues of arginine, lysine, histidine, glutamic acid, or a combination thereof. In an aspect, a cationic cell penetrating peptide comprises Tat or Arg9.

The cell penetrating peptide can comprise naturally-occurring and/or non-natural amino acid residues. The term “naturally-occurring amino acid” refers to alanine (A or Ala), arginine (R or Arg), asparagine (N or Asn), aspartic acid (D or Asp), cysteine (C or Cys), glutamic acid (E or Glu), glutamine (Q or Gln), glycine (G or Gly), histidine (H or His), isoleucine (I or Ile), leucine (L or Leu), lysine (K or Lys), methionine (M or Met), phenylalanine (F or Phe), proline (P or Pro), serine (S or Ser), threonine (T or Thr), tryptophan (W or Trp), tyrosine (Y or Tyr), and valine (V or Val). “Non-natural amino acids” (i.e., amino acids that do not occur naturally) include, by way of non-limiting example, homoserine, homoarginine, citrulline, phenylglycine, taurine, iodotyrosine, seleno-cysteine, norleucine (“Nle”), norvaline (“Nva”), beta-alanine, L- or D-naphthalanine, ornithine (“Orn”), and the like. Peptides can be designed and optimized for enzymatic cleavage by a particular enzyme, for example, a tumor -associated protease, cathepsin B, C and D, or a plasmin protease.

Amino acids also include the D-forms of natural and non-natural amino acids. “D-” designates an amino acid having the “D” (dextrorotary) configuration, as opposed to the configuration in the naturally occurring (“L-”) amino acids. Natural and non-natural amino acids can be purchased commercially (Sigma Chemical Co., Advanced Chemtech) or synthesized using methods known in the art.

In some aspects, the cell penetrating peptide or a segment thereof can comprise low sequence diversity, e.g., comprising 50% or greater of a single amino acid, such as argininyl, cysteinyl, prolinyl, or lysinyl. In an aspect, the cell penetrating peptide or a segment thereof can be argininyl-rich, cysteinyl-rich, prolinyl-rich, or lysinyl-rich, in which the sequence or segment comprises 50% or greater of that particular amino acid.

In some aspects, the cell penetrating peptide, a segment thereof, or a side chain thereof can be classified as hydrophobic. In an aspect, hydrophobicity can be introduced by including one or more (e.g., 1, 2, 3, 4, 5, 6, 7, 8, 9, or 10, etc.) residues of tryptophan, phenylalanine, tyrosine, leucine, or a combination thereof. For example, the amino acid sequences WWWWW (SEQ ID NO: 1094) and FFLIPKG (SEQ ID NO: 1095) and the cell penetrating peptide gH 625 (a peptidyl of the sequence HGLASTLTRWAHYNALIRAF (SEQ ID NO: 1113)) are considered hydrophobic.

In some aspects, the cell penetrating peptide can comprise three or more argininyl moieties (e.g., 3, 4, 5, 6, 7, 8, 9, 10, 11, 12, 13, 14, 15, or 16 argininyl moieties). In an aspect, the cell penetrating peptide can comprise three (Arg3), six (Arg6), eight (Args), or nine (Arg9) argininyl moieties. In some aspects, the cell penetrating peptide further comprises at least one amino acid other than argininyl, such as cysteinyl, glycinyl, or a combination thereof. In some aspects, the at least one amino acid other than argininyl is cysteinyl, glycinyl, lysinyl, glutaminyl, isoleucinyl, tryptophanyl, phenylalaninyl, valinyl, threoninyl, serinyl, or a combination thereof.

In some aspects, the cell penetrating peptide comprises a cyclic peptide, TAT (a peptidyl of the sequence YGRKKRRQRRR (SEQ ID NO: 1096)), Antp (antennapedia; a peptidyl of the sequence RQIKIWFQNRRMKWKK (SEQ ID NO: 1097)), DPV3 (a peptidyl of the sequence RKKRRRESRKKRRRES (SEQ ID NO: 1098)), DPV6 (a peptidyl of the sequence GRPRESGKKRKRKRLKP (SEQ ID NO: 1099)), penetratin (a peptidyl of the sequence RQIKIWFQNRRMKWKK (SEQ ID NO: 1100)), R9-TAT (a peptidyl of the sequence GRRRRRRRRRPPQ (SEQ ID NO: 1101)), pVEC(a peptidyl of the sequence LLIILRRRIRKQAHAHSK (SEQ ID NO: 1102)), ARF (19-31)(a peptidyl of the sequence RVRVFVVHIPRLT (SEQ ID NO: 1103)), MPG (a peptidyl of the sequence GALFLGFLGAAGSTMGAWSQPKKKRKV (SEQ ID NO: 1104)), MAP (a peptidyl of the sequence KLALKLALKALKAALKLA (SEQ ID NO: 1105)), transportan (a peptidyl of the sequence GWTLNSAGYLLGKINLKALAALAKKIL (SEQ ID NO: 1106)), Bip4 (a peptidyl of the sequence VSALK (SEQ ID NO: 1107)), C105Y (a peptidyl of the sequence CSIPPEVKFNPFVYLI (SEQ ID NO: 1108)), melittin (a peptidyl of the sequence GIGAVLKVLTTGLPALISWIKRKRQQ (SEQ ID NO: 1109)), gH625 (a peptidyl of the sequence HGLASTLTRWAHYNALIRAF (SEQ ID NO: 1110)), Pep-1 (a peptidyl of the sequence KETWWETWWTEWSQPKKKRKV (SEQ ID NO: 1111)), SAP (sweet arrow peptide; a peptidyl of the sequence (VRLPPP) 3 (SEQ ID NO: 1112)), gH 625 (a peptidyl of the sequence HGLASTLTRWAHYNALIRAF (SEQ ID NO: 1113)), crot (27-29)(a peptidyl of the sequence KMDCRWRWKCCKK (SEQ ID NO: 1114)), R6/W3 (a peptidyl of the sequence RRWWRWRR (SEQ ID NO: 1115)), TP10 (a peptidyl of the sequence AGYLLGKINLKALAALAKKIL (SEQ ID NO: 1116)), CADY (a peptidyl of the sequence Ac-GLWRALWRLLRSLWRLLWRA-cysteamide (SEQ ID NO: 1117)), sC18 (a peptidyl of the sequence GLRKRLRKFRNKIKEK (SEQ ID NO: 1118)), YTA4 (a peptidyl of the sequence IAWVKAFIRKLRKGPLG (SEQ ID NO: 1119)), CyLoP-1 (a peptidyl of the sequence CRWRWKCCKK (SEQ ID NO: 1120)), GALA (a peptidyl of the sequence WEAALAEALAEALAEHLAEALAEALEALAA (SEQ ID NO: 1121)), or R6 (a peptidyl of the sequence RRRRRR (SEQ ID NO: 1122)). Other suitable examples of a cell penetrating peptide are disclosed in Kalafatovic et al., Molecules, 22 (11), 1929 (2017), which is incorporated herein by reference in its entirety.

In some aspects, the cell penetrating peptide comprises a cyclic peptide, TAT (a peptidyl of the sequence YGRKKRRQRRR (SEQ ID NO: 1096)), or Antp (antennapedia; a peptidyl of the sequence RQIKIWFQNRRMKWKK (SEQ ID NO: 1097)).

In some aspects, the cell penetrating peptide is a cyclic peptide. Examples of cyclic peptides include, e.g., cyclic Tat (cTAT, a peptide of sequence KRRRGRKKRRE (wherein K and E are connected to form a cyclic peptide)(SEQ ID NO: 1123)), cyclic [WR] +, cyclic [WR] 5, cyclic [WH] s, cyclic [KW] +, cyclic [KW] s, cyclic [CR] +, cyclic [CR] s, cyclic [HR] 5, cyclic [W (RW)+K], cyclic [C(RW)+K], C16-[r₁₂] (r=arginine), or cyclic sC18. Other suitable examples of a cyclic cell penetrating peptide are disclosed in Park et al., Molecular Pharmaceutics, 16, 3727-3743 (2019), which is incorporated herein by reference in its entirety.

In some aspects, the cell penetrating peptide is classified as primary amphipathic (e.g., Pep-1, TP10), secondary amphipathic (e.g., penetratin, R6/W3, CADY, sC18), or non-amphipathic.

### Other Cleavable Linkages

The construct of Formula I or II comprises a cell penetrating peptide. In some aspects, L₁ is present in Formula I or II and comprises the cell penetrating peptide. In some aspects, L₂ is present in Formula I or II and comprises the cell penetrating peptide. In some aspects, L₃ is present in Formula I and comprises the cell penetrating peptide.

In some aspects, at least one (i.e., 1 or 2 of L₁, L₂, and L₃) cleavable linkage of L₁, L₂, and L₃ that does not comprise the cell penetrating peptide is present and is a cleavable linkage comprising a phosphodiester bond, a disulfido, a polypeptidyl, a polynucleotidyl, a pyrophosphato, or a silyl ether, or a combination thereof.

In some aspects, the at least one (i.e., 1 or 2 of L₁, L₂, and L₃) cleavable linkage that does not comprise the cell penetrating peptide is a cleavable linkage comprising a phosphodiester bond.

In some aspects, the at least one (i.e., 1 or 2 of L₁, L₂, and L₃) cleavable linkage that does not comprise the cell penetrating peptide is a cleavable linkage comprising a disulfido bond.

In some aspects, the at least one (i.e., 1 or 2 of L₁, L₂, and L₃) cleavable linkage that does not comprise the cell penetrating peptide comprises a polynucleotidyl. In some aspects, the polynucleotidyl is a trinucleotidyl or higher, such as a tetranucleotidyl or higher, a pentanucleotidyl or higher, a hexanucleotidyl or higher, a heptanucleotidyl or higher, an octanucleotidyl or higher, a nonanucleotidyl or higher, or a decanucleotidyl or higher. Typically, the polynucleotidyl is not longer than 50 nucleotides in length (e.g., 45 nucleotides or fewer, 40 nucleotides or fewer, 35 nucleotides or fewer, 30 nucleotides or fewer, 25 nucleotides or fewer, 20 nucleotides or fewer, 15 nucleotides or fewer, 14 nucleotides or fewer, 13 nucleotides or fewer, 12 nucleotides or fewer, 11 nucleotides or fewer, 10 nucleotides or fewer, 9 nucleotides or fewer, 8 nucleotides or fewer, 7 nucleotides or fewer, 6 nucleotides or fewer, 5 nucleotides or fewer, 4 nucleotides or fewer, or 3 nucleotides). In some aspects, L₁ comprises a tetranucleotidyl.

In general, the polynucleotidyl will comprise adenylic acid (AMP, dAMP), guanylic acid (GMP, dGMP), cytidylic acid (CMP, dCMP), thymidylic acid (dTMP), uridylic acid (UMP), or any combination thereof. In an example, the polynucleotidyl is a tetranucleotidyl comprising dTdTdTdT, wherein dT is thymidylic acid.

In some aspects, the at least one (i.e., 1 or 2 of L₁, L₂, and L₃) cleavable linkage that does not comprise the cell penetrating peptide is a cleavable linkage comprising a pyrophosphato bond. In some aspects, a pyrophosphato bond is of the formula

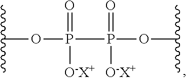

wherein X+ is a monovalent cation, such as a proton (H⁺) or a Group I cation (e.g., Li⁺, Na⁺, K⁺, or Rb⁺) and [symbol] denotes connectivity to AM, BAM, or a spacer.

In some aspects, the at least one (i.e., 1 or 2 of L₁, L₂, and L₃) cleavable linkage that does not comprise the cell penetrating peptide is a cleavable linkage comprising a silyl ether. In some aspects, a silyl ether bond comprises —OSiR¹R²O—, wherein R¹ and R² are the same or different and each is C₁₋₈ alkyl (e.g., methyl, ethyl, n-propyl, isopropyl, n-butyl) or aryl (e.g., phenyl). In an example, R¹ and R² are both isopropyl.

In some aspects, two of L₁, L₂, and L₃ are the same and each is a cleavable linkage comprising a phosphodiester bond, a disulfido, or a polypeptidyl that is not a cell penetrating peptide. In some aspects, one of L₁, L₂, and L₃ is a cleavable linkage comprising a phosphodiester bond, and the other cleavable linkage that does not comprise the cell penetrating peptide is a cleavable linkage comprising a disulfido bond. In some aspects, L₁ is a cleavable linkage comprising a phosphodiester bond, L₂ is a cleavable linkage comprising a disulfido bond, and L₃ comprises the cell penetrating peptide. In some aspects, L₁ is a cleavable linkage comprising a disulfido bond, L₂ is a cleavable linkage comprising a phosphodiester bond, and and L₃ comprises the cell penetrating peptide. In some aspects, L₁ comprises the cell penetrating peptide, L₂ is a cleavable linkage comprising a disulfido or phosphodiester bond, and L₃ is absent. In some aspects, L₁ is a cleavable linkage comprising a disulfido or phosphodiester bond, L₂ comprises the cell penetrating peptide, and L₃ is absent. In some aspects, L₃ comprises the cell penetrating peptide and both L₁ and L₂ are absent.

In some aspects, the linker combination not comprising the cell penetrating peptide can comprise a cleavable likage that is cleavable by intracellular or extracellular enzymes, e.g., a protease, an esterase, a nuclease, an amidase. The range of enzymes that can cleave a specific linker in a linker combination depends on the specific bonds and chemical structure of the linker. Accordingly, peptidic linkers can be cleaved, e.g., by a peptidase, linkers containing ester linkages can be cleaved, e.g., by an esterase; linkers containing amide linkages can be cleaved, e.g., by an amidase; etc.

In some aspects, the linker combination not comprising the cell penetrating peptide comprises an additional cleavable linkage that is a protease cleavable linkage, i.e., a linker that can be cleaved by an endogenous protease. Only certain peptides are readily cleaved inside or outside cells. See, e.g., Trout et al., Proc. Natl. Acad. Sci. USA, 79:626-629 (1982) and Umemoto et al., Int. J. Cancer, 43:677-684 (1989), the contents of which are incorporated herein by reference in their entireties. Protease cleavable linkers can contain one or more cleavable sites composed of a-amino acid units and peptidic bonds, which chemically are amide bonds between the carboxylate of one amino acid and the amino group of a second amino acid. Other amide bonds, such as the bond between a carboxylate and the α-amino acid group of lysine, are understood not to be peptidic bonds and are considered non-cleavable.

In some aspects, the additional protease cleavable linker can comprise a peptide comprising 1 to 100 amino acid residues, i.e., a peptide that is not considered to be a cell penetrating peptide. In some aspects, the peptide linker can comprise at least two, at least three, at least four, at least five, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 amino acids. In these aspects, the peptide allows for cleavage of the linker by a protease, thereby facilitating release of the biologically active molecule upon exposure to intracellular proteases, such as lysosomal enzymes (Doronina et al., Nat. Biotechnol., 21:778-784 (2003)). Exemplary peptides include, but are not limited to, dipeptides, tripeptides, tetrapeptides, pentapeptides, and hexapeptides.

A peptide can comprise naturally-occurring and/or non-natural amino acid residues. The term “naturally-occurring amino acid” refers to alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val).

“Non-natural amino acids” (i.e., amino acids that do not occur naturally) include, by way of non-limiting example, homoserine, homoarginine, citrulline, phenylglycine, taurine, iodotyrosine, seleno-cysteine, norleucine (“Nle”), norvaline (“Nva”), beta-alanine, L- or D-naphthalanine, ornithine (“Orn”), and the like. Peptides can be designed and optimized for enzymatic cleavage by a particular enzyme, for example, a tumor -associated protease, cathepsin B, C and D, or a plasmin protease.

Amino acids also include the D-forms of natural and non-natural amino acids. “D-” designates an amino acid having the “D” (dextrorotary) configuration, as opposed to the configuration in the naturally occurring (“L-”) amino acids. Natural and non-natural amino acids can be purchased commercially (Sigma Chemical Co., Advanced Chemtech) or synthesized using methods known in the art.

Exemplary dipeptides include, but are not limited to, valine-glycine (Val-Gly), glycine-glycine (Gly-Gly), cyclobutane-1,1-dicarboxamide-citrulline (cBu-Cit), valine-alanine (Val-Ala), valine-citrulline (Val-Cit), phenylalanine-lysine (Phe-Lys), N-methyl-valine-citrulline, cyclohexylalanine-lysine, and beta-alanine-lysine. Exemplary tripeptides include, but are not limited to, glutamic acid-valine-citrulline (Glu-Val-Cit), aspartic acid-valine-citrulline (Asp-Val-Cit), serine-valine-citrulline (Ser-Val-Cit), alanine-phenylalanine-lysine (Ala-Phe-Lys), lysine-valine-citrulline (Lys-Val-Cit), alanine-alanine-asparagine (Ala-Ala-Asn), glycine-valine-citrulline (Gly-Val-Cit) and glycine-glycine-glycine (Gly-Gly-Gly). An exemplary higher peptide includes, but is not limited to, glycine-glycine-glycine-valine-citrulline (Gly-Gly-Gly-Val-Cit).

In some aspects, the at least one (i.e., 1 or 2 of L₁, L₂, and L₃) cleavable linkage that does not comprise the cell penetrating peptide is a cleavable linkage that can comprise a peptidyl, i.e., a peptidyl that is not considered a cell penetrating peptide. Suitable examples of such cleavable peptide linkages include alanine-alanine-asparagine, valine-glycine, glycine-glycine, glutamic acid-valine-citrulline, aspartic acid-valine-citrulline, serine-valine-citrulline, lysine-valine-citrulline, glycine-glycine-glycine-valine-citrulline, cyclobutane-1,1-dicarboxamide-citrulline, or alanine-phenylalanine-lysine). In some aspects, L₁, L₂, and/or L₃ can comprise a peptidyl comprising alanine-alanine-asparagine. In some aspects, the peptidyl comprises valine-glycine. In some aspects, the peptidyl comprises glycine-glycine. In some aspects, the peptidyl comprises glutamic acid-valine-citrulline. In some aspects, the peptidyl comprises aspartic acid-valine-citrulline. In some aspects, the peptidyl comprises serine-valine-citrulline. In some aspects, the peptidyl comprises lysine-valine-citrulline. In some aspects, the peptidyl comprises glycine-glycine-glycine-valine-citrulline. In some aspects, the peptidyl comprises cyclobutane-1,1-dicarboxamide-citrulline (cBu-Cit). In some aspects, the peptidyl comprises alanine-phenylalanine-lysine.

In some aspects, the peptide linker is synthetic, i.e., non-naturally occurring. In one aspect, a peptide linker includes peptides (or polypeptides)(e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In some aspects, the linker comprises a glycine/serine linker. In some aspects, the peptide linker is glycine/serine linker according to the formula [(Gly)ₙ-Ser] m, where n is any integer from 1 to 100 and m is any integer from 1 to 100. In other aspects, the glycine/serine linker is according to the formula [(Gly) x-(Ser);] 2, wherein x in an integer from 1 to 4, y is 0 or 1, and z is an integer from 1 to 50. In some aspects, the peptide linker comprises the sequence Glyn, where n can be an integer from 1 to 100. In some aspects, the peptide linker can comprise the sequence (GlyAla) n, wherein n is an integer between 1 and 100. In other aspects, the peptide linker can comprise the sequence (GlyGlySer) n, wherein n is an integer between 1 and 100.

In some aspects, the protease-cleavable linker comprises a cleavage site for a protease, e.g., neprilysin (common acute lymphoblastic leukemia antigen (CALLA) or CD10), thimet oligopeptidase (TOP), leukotriene A4 hydrolase, endothelin converting enzymes, ste24 protease, neurolysin, mitochondrial intermediate peptidase, interstitial collagenases, collagenases, stromelysins, macrophage elastase, matrilysin, gelatinases, meprins, procollagen C-endopeptidases, procollagen N-endopeptidases, ADAMs and ADAMTs (A Disintegrin and Metalloproteinase with Thrombospondin) metalloproteinases, myelin associated metalloproteinases, enamelysin, tumor necrosis factor α-converting enzyme, insulysin, nardilysin, mitochondrial processing peptidase, magnolysin, dactylysin-like metalloproteases, neutrophil collagenase, matrix metallopeptidases, membrane-type matrix metalloproteinases, SP₂ endopeptidase, prostate specific antigen (PSA), plasmin, urokinase, human fibroblast activation protein (FAPa), trypsin, chymotrypsins, caldecrin, pancreatic elastases, pancreatic endopeptidase, enteropeptidase, leukocyte elastase, myeloblasts, chymases, tryptase, granzyme, stratum corneum chymotryptic enzyme, acrosin, kallikreins, complement components and factors, alternative-complement pathway c3/c5 convertase, mannose-binding protein-associated serine protease, coagulation factors, thrombin, protein c, u and t-type plasminogen activator, cathepsin G, hepsin, prostasin, hepatocyte growth factor -activating endopeptidase, subtilisin/kexin type proprotein convertases, furin, proprotein convertases, prolyl peptidases, acylaminoacyl peptidase, peptidyl-glycaminase, signal peptidase, n-terminal nucleophile aminohydrolases, 20s proteasome, γ-glutamyl transpeptidase, mitochondrial endopeptidase, mitochondrial endopeptidase Ia, htra2 peptidase, matriptase, site 1 protease, legumain, cathepsins, cysteine cathepsins, calpains, ubiquitin isopeptidase T, caspases, glycosylphosphatidylinositoliprotein transamidase, cancer procoagulant, prohormone thiol protease, γ-glutamyl hydrolase, bleomycin hydrolase, seprase, cathepsin B, cathepsin D, cathepsin L, cathepsin M, proteinase K, pepsins, chymosyn, gastricsin, renin, yapsin and/or mapsins, Prostate-Specific antigen (PSA), or any Asp-N, Glu-C, Lys-C or Arg-C proteases in general. See, e.g., Caculitan et al., Cancer Res., 77 (24): 7027-7037 (2017), which is herein incorporated by reference in its entirety.

### Enzymatic Cleavable Linkers: Esterase Cleavable Linkers

Some additional cleavable linkages can be cleaved by esterases (“esterase cleavable linkers”). Only certain esters can be cleaved by esterases and amidases present inside or outside of cells. Examples of ester-based cleavable linking groups include, but are not limited to, esters of alkylene, alkenylene, and alkynylene groups. The ester cleavable linking group has the general formula —C(O)O— or —OC(O)—.

### Enzymatic Cleavable Linkers: Phosphatase Cleavable Linkers

In some aspects, a linker combination can include an additional cleavable linker that can be a phosphate-based cleavable linking group that can be cleaved by an agent that degrades or hydrolyzes phosphate groups, such as intracellular phosphatase. Examples of cleavable phosphate-based linkages are —O—P(O)(ORₖ)O—, —O—P(S)(ORₖ)—O—, —O—P(S)(SRK)—O—, —S—P(O)(ORₖ)—O—, —O—P(O)(ORₖ)—S—, —S—P(O)(ORₖ)—S—, —O—P(S)(ORₖ)—S—, —SP(S) (ORₖ)—O—, —OP(O)(Rₖ)—O—, —OP(S)(Rₖ)—O—, —SP(O)(Rₖ)—O—, —SP(S)(Rₖ)—O—, —SP(O)(Rₖ)—S—, or —OP(S)(Rₖ)—S—. In various aspects, Rₖ is any of the following: NH₂, BH₃, CH₃, C₁₋₆ alkyl, C₆₋₁₀ aryl, C₁₋₆ alkoxy and C₆₋₁₀ aryloxy. In some aspects, C₁₋₆ alkyl and C₆₋₁₀ aryl are unsubstituted. Further non-limiting examples are —O—P(O)(OH)—O—, —O—P(S)(OH)—O—, —O—P(S)(SH)—O—, —S—P(O)(OH)—O—, O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —SP(S)(H)—O—, —SP(O)(H)—S—, —OP(S)(H)—S—, and —O—P(O)(OH)—O—.

### Photoactivated Cleavable Linkers

In some aspects, the combination linker comprises an additional cleavable linkage comprising a photoactivated cleavable linkage, such as a nitrobenzyl linkage or a linker comprising a nitrobenzyl reactive group.

In some aspects, the additional cleavable linkage can comprise a dinucleotidyl (e.g., Val-Cit dipeptidyl) bond, a trinucleotidyl bond, a disulfido (—S—S—) bond, an imino bond, a thioketal bond, or any combination thereof.

In some aspects, the additional cleavable linkage comprises valine-alanine-p-aminobenzylcarbamato or valine-citrulline-p-aminobenzylcarbamato.

### Self-immolative Linker

In some aspects, the at least one (i.e., 1 or 2 of L₁, L₂, and L₃) cleavable linkage that does not comprise the cell penetrating peptide is a cleavable linkage that can comprise or consist of a self-immolative linker. The term “self-immolative linker” as used herein refers to a spacer that will spontaneously separate from a first moiety (e.g., a biologically active molecule, linkage, spacer, or anchoring moiety) if its bond to a second moiety (e.g., a biologically active molecule, linkage, spacer, or anchoring moiety) is cleaved.

In some aspects, the self-immolative linker is, e.g., a p-aminobenzyl (pAB) derivative, such as a p-aminobenzyl carbamate (pABC), a p-amino benzyl ether (PABE), a p-amino benzyl carbonate, or a combination thereof. In certain aspects, the self-immolative linker comprises an aromatic group or a heterocyclic group. In some aspects, the aromatic group is selected from the group consisting of benzyl, cinnamyl, naphthyl, and biphenyl. In other aspects, the aromatic group comprises at least one (e.g., 1, 2, 3, or 4) substituent (e.g., F, Cl, I, Br, OH, methyl, methoxy, NO₂, NH₂, NO₃, NHCOCH₃, N(CH₃)₂, NHCOCF₃, alkyl, haloalkyl, carboxylato, sulfato, sulfamato, and/or sulfonato). In other aspects, at least one C in the aromatic group is substituted with N, O, or C—R*, wherein R* is independently selected from H, F, Cl, I, Br, OH, methyl, methoxy, NO₂, NH₂, NO₃⁻, NHCOCH₃, N (CH₃)₂, NHCOCF₃, alkyl, haloalkyl, carboxylato, sulfato, sulfamato, and sulfonato.

In some aspects, L₁ comprises or consists of a self-immolative linker (e.g., p-aminobenzyl carbamate, pABC). In some aspects, L₂ comprises or consists of a self-immolative linker (e.g., p-aminobenzyl carbamate, pABC). In some aspects, L₃ comprises or consists of a self-immolative linker (e.g., p-aminobenzyl carbamate, pABC).

The aromatic ring of the aminobenzyl group can optionally be substituted with one or more (e.g., R¹ and/or R²) substituents on the aromatic ring, which replace a hydrogen that is otherwise attached to one of the four non-substituted carbons that form the ring. As used herein, the symbol “R^{X}” (e.g., R¹, R², R³, R⁴) is a general abbreviation that represents a substituent group as described herein. Substituent groups can improve the self-immolative ability of the p-aminobenzyl group (see, e.g., Hay et al., J. Chem Soc., Perkin Trans., 1:2759-2770 (1999); and Sykes et al., J. Chem. Soc., Perkin Trans., 1:1601-1608 (2000)). Substituent groups in self-immolative, for example, R¹ and/or R² substituents in a p-aminobenzyl self-immolative linker as discuss above can include, e.g., alkyl, alkylenyl, alkenyl, alkynyl, alkoxy, alkylamino, alkylthio, heteroalkyl, cycloalkyl, heterocycloalkyl, aryl, arylalkyl, aryloxy, heteroaryl, etc. When a compound of the present disclosure includes more than one substituent, then each of the substituents is independently chosen. Where substituent groups in the self-immolative linker are specified by their conventional chemical formulae, written from left to right, they equally encompass the chemically identical substituents, which would result from writing the structure from right to left. For example, “—CH₂O—” is intended to also recite “—OCH₂—”.

In some aspects, the self-immolative linker can comprise or consist of a cinnamyl, a naphthyl, a biphenyl, a heterocyclyl, a homoaromatic group, coumarinyl, furanyl, thiophenyl, thiazolyl, oxazolyl, isoxazolyl, pyrrolyl, pyrazolyl, pyridinyl, imidazonyl, triazolyl, or any combination thereof. In some aspects, L₁ comprises a cleavable linker selected from the group consisting of a cinnamyl, a naphthyl, a biphenyl, a heterocyclyl, a homoaromatic group, coumarinyl, furanyl, thiophenyl, thiazolyl, oxazolyl, isoxazolyl, pyrrolyl, pyrazolyl, pyridinyl, imidazonyl, triazolyl, or any combination thereof. In some aspects, L₂ comprises a cleavable linker selected from the group consisting of a cinnamyl, a naphthyl, a biphenyl, a heterocyclyl, a homoaromatic group, coumarinyl, furanyl, thiophenyl, thiazolyl, oxazolyl, isoxazolyl, pyrrolyl, pyrazolyl, pyridinyl, imidazonyl, triazolyl, or any combination thereof. In some aspects, L₃ comprises a cleavable linker selected from the group consisting of a cinnamyl, a naphthyl, a biphenyl, a heterocyclyl, a homoaromatic group, coumarinyl, furanyl, thiophenyl, thiazolyl, oxazolyl, isoxazolyl, pyrrolyl, pyrazolyl, pyridinyl, imidazonyl, triazolyl, or any combination thereof.

In some aspects, the self-immolative linker connects a biologically active molecule BAM (e.g., an ASO) to a protease-cleavable peptidyl (e.g., Val-Cit). In specific aspects, the carbamate group of a pABC self-immolative linker is connected to an amino group of a biologically active molecule BAM (e.g., ASO), and the amino group of the pABC self-immolative linker is connected to a protease-cleavable peptidyl.

Self-immolative elimination can take place, e.g., via 1,4 elimination, 1,6 elimination (e.g., pABC), 1,8 elimination (e.g., p-amino-cinnamyl alcohol), β-elimination, cyclization-elimination (e.g., 4-aminobutanol ester and ethylenediamines), cyclization/lactonization, cyclization/lactolization, etc. See, e.g., Singh et al., Curr. Med. Chem., 15:1802-1826 (2008); and Greenwald et al., J. Med. Chem., 43:475-487 (2000).

In some aspects, a linker combination disclosed herein can comprise more than one self-immolative linker in tandem, e.g., two or more pABC units. In some aspects, a linker combination disclosed herein can comprise a self-immolative linker (e.g., a p-aminobenzylalcohol or a hemithioaminal derivative of p-carboxybenzaldehyde or glyoxilic acid) linked to a fluorigenic probe.

In some aspects, a cleavable linkage disclosed herein, e.g., an L₁, L₂, or L₃ linkage or any combination thereof that does not comprise the cell penetrating peptide can comprise or consist of a linkage (generally a dipeptide or tripeptide linker) having the formula:

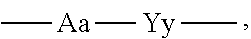

wherein each -A- is independently an amino acid unit or a combination thereof, a is independently an integer from 1 to 15; -Y- is a spacer unit, as described herein, and y is 0, 1, or 2. In some aspects, Li comprises or consists of the -Aa-Yy- linker. In some aspects, L₂ comprises or consists of the Aa-Yy-linker. In some aspects, L₃ comprises or consists of the -Aa-Yy- linker.

In some aspects -Aa- is a dipeptidyl, a tripeptidyl, a tetrapeptidyl, a pentapeptidyl, or a hexapeptidyl. In some aspects wherein a is 2 (i.e., a dipeptidyl cleavable linker), -Aa-is selected from the group consisting of valine-alanine, valine-citrulline, phenylalanine-lysine, N-methylvaline-citrulline, cyclohexylalanine-lysine, and beta-alanine-lysine. In some aspects wherein a is 3 (i.e., a tripeptidyl cleavable linker), -Aa-is glutamic acid-valine-citrulline. In some aspects, y is 1, i.e., the cleavable linker of formula -Aa-Yy-comprises a single spacer. In some aspects, the cleavable linker of formula -Aa-Yy-can comprises more than one spacer, e.g., two spacers. In some aspects, the two spacers are the same. In other aspects, the two spacers are different. In some aspects, the single spacer can be cleavable or non-cleavable. In some aspects, the first spacer (proximal to Aa) can be cleavable and the second spacer (distal to Aa) can be non-cleavable. In some aspects, the first spacer (proximal to Aa) can be non-cleavable and the second spacer (distal to Aa) can be cleavable. In some aspects, both spacers can be non-cleavable. In some aspects, both spacer can be cleavable.

In some aspects, a-Y-spacer can be a self-immolative spacer, e.g., p-aminobenzylcarbamate (pABC). In some aspects, -Yy- has the formula:

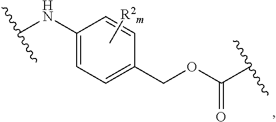

wherein each R² is independently C₁₋₈ alkyl, —O—(C₁₋₈ alkyl), halo, nitro, or cyano; and m is 0 or an integer from 1 to 4 (i.e., 0, 1, 2, 3, or 4). In some aspects, m is 0, 1, or 2. In some aspects, m is 0.

In some aspects, a cleavable linkage disclosed herein, e.g., an L₁, L₂, or L₃ linkage or any combination thereof comprises or consists of valine-alanine-p-aminobenzylcarbamate or valine-citrulline-p-aminobenzylcarbamate.

In some aspects, -Y-is a non self-immolative spacer, e.g., a peptidyl spacer. Peptidyl spacers are generally glycine (Gly) based spacers or glycine-serine (Gly/Ser) based spacers. In some aspects, the Y-peptidyl spacer comprises or consists of Gly- or Gly-Gly-. Other peptide spacers such as the (Gly₄Ser) n spacer are described herein.

### Spacers

Typically, the cleavable linkage L₁, L₂, and/or L₃ is part of a spacer (e.g., SP₁, SP₂, SP₃, and/or SP₄) between the anchoring moiety AM and the biologically active molecule BAM to provide the optimal spacing between the anchoring moiety or moieties AM and the biologically active molecule or molecules BAM. For example, in the case of an antisense oligonucleotide (ASO), one goal of a combination of linker and spacers is to reduce steric hindrance and position the ASO so it can interact with a target nucleic acid, e.g., an mRNA or an miRNA. Other goals can include, for example, increasing the loading efficiency of the EV (e.g., an exosome), increasing the number of BAM per EV, increasing the surface density of BAM on the EV, increasing the potency of the BAM after incorporate to the EV, or any combination thereof. In the context of the present disclosure, the term “linker combination” refers to the combination of a cleavable linkage L₁, L₂, and/or L₃ and one or more spacers that forms the construct of Formula I or II disclosed herein.

In some aspects, SP₁ is present in Formula I or II. In some aspects, SP₂ is present in Formula I or II. In some aspects, SP₃ is present in Formula I or II. In some aspects, SP₄ is present in Formula I. In some aspects, SP₁ and SP₂ are both present in Formula I or II. In some aspects, SP₁ and SP₃ are both present in Formula I or II. In some aspects, SP₁ and SP₄ are both present in Formula I. In some aspects, SP₂ and SP₃ are both present in Formula I or II. In some aspects, SP₂ and SP₄ are both present in Formula I. In some aspects, SP₃ and SP₄ are both present in Formula I. In some aspects, SP₁, SP₂, and SP₃ are present in Formula I or II. In some aspects, SP₁, SP₂, and SP₄ are present in Formula I. In some aspects, SP₂, SP₃, and SP₄ are present in Formula I. In some aspects, SP₁, SP₂, SP₃, and SP₄ are present in Formula I.

In some aspects, the cleavable linkage L₁, L₂, and/or L₃ is part of a spacer moiety, i.e., SP₁, SP₂, SP₃, and/or SP₄, prior to conjugation to AM and/or BAM. In some aspects, L₁ is part of SP₂ and AM is part of SP₁ prior to conjugation to BAM. In some aspects, L₁ and L₂ are part of both SP₁ and SP₂ and AM and BAM is part of SP₃ prior to conjugation to one another. In some aspects, L₁ and L₂ are part of SP₂, SP₃, and BAM and AM is part of SPI prior to conjugation to one another. In some aspects, L₁ and L₂ are part of both SP₁ and SP₂ prior to conjugation to AM and BAM. In some aspects, L₁ is part of SP₁ and AM and L₂ is part of SP₂ prior to conjugation to BAM. In some aspects, L₁ is part of both SP₁, SP₂, and AM and L₂ is part of SP₃ and BAM prior to conjugation to AM.

In some aspects, SP₁, SP₂, SP₃, and SP₄ of Formula I or II are the same or different and each comprises an alkylenyl, a polyoxyalkylenyl, a succinimido, a maleimido, an aryl (e.g., 1,2-phenyl, 1,3-phenyl, or 1,4-phenyl), an ether (—O—), a carbonyl (—C(O)—), a carboxy (—C(O)O- or —OC(O)—), a carbamoyl (—OC(O)NR— or —NRC(O)O—), a thioether (—S—), a sulfo (—SO₂—), a thiocarbonyl (—C(S)—), a thiocarbamoyl (—OC(S)NR— or —NRC(S)O—), a thiosuccinimido, an amino (—NR—), an amido (—C(O)NR— or —NRC(O)—), a hydrazido (—NH—NH—), a phosphorothioato (—OP(═S)(OH)O—), a 1,2,3-triazolyl, a dibenzoylcyclooctenyl, a bicyclononenyl, a p-aminobenzoyl, a p-aminobenzylcarbamato, or a combination thereof, and at least one of SP₁, SP₂, SP₃, and SP₄ is present. The following moieties are associated with the provided structures

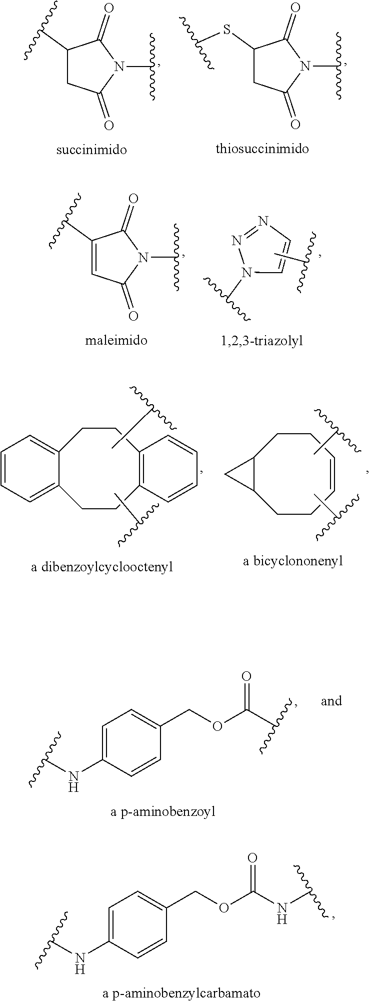

wherein [symbol] denotes connectivity to AM, BAM, L₁, L₂, L₃, or the remainder of the spacer.

In some aspects, at least one of SP₁, SP₂, SP₃, and SP₄ comprises C₁₋₈ alkylenyl, polyoxyalkenyl, a maleimido, a carbamoyl, a thio, an amido, a 1,2,3-triazolyl, a dibenzoylcyclooctenyl, a bicyclononenyl, a p-aminobenzoyl, a p-aminobenzylcarbamato, or a combination thereof. In some aspects, at least one of SP₁, SP₂, SP₃, and SP₄ (e.g., SP₁ and SP₂, SP₂ and SP₃, or SP₁ and SP₃) comprises C₁₋₆ alkylenyl (i.e., C₁ alkylenyl, C₂ alkylenyl, C₃ alkylenyl, C₄ alkylenyl, C₅ alkylenyl, or C₆ alkylenyl), as described herein. In some aspects, at least one of SP₁ and SP₂ comprises a polyoxyalkylenyl (e.g., a glycol-based spacer) that comprises 2 to 15 —OCH₂CH₂-repeat units, as described herein. In some aspects, the polyoxyalkylenyl (e.g., a glycol-based spacer) that comprises 4 (TEG) or 6 (HEG) —OCH₂CH₂-repeat units, as described herein. In some aspects, at least one of SP₁, SP₂, SP₃, and SP₄ (e.g., SP₁, SP₂, and SP₃) comprises C₁₋₆ alkylenyl or a polyoxyalkylenyl (e.g., a glycol-based spacer) that comprises 2 to 15 —OCH₂CH₂-repeat units, as described herein, and further comprises comprises a carbamoyl, an amino, an amido, a thiosuccinimido, 1,2,3-triazolydibenzoylcyclooctenyl, a 1,2,3-triazolylbicyclononenyl, or a combination thereof, wherein the 1,2,3-triazolydibenzoylcyclooctenyl has the structure

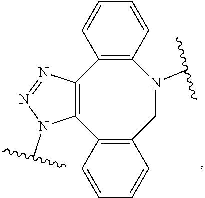

and
the 1,2,3-triazolylbicyclononenyl has the structure:

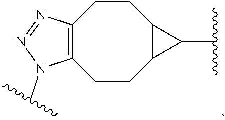

wherein [symbol] denotes connectivity to AM, BAM, L₁, L₂, L₃, or the remainder of the spacer.

### Alkylenyl Spacer

In some aspects, a spacer (e.g., SP₁, SP₂, SP₃, and/or SP₄) in Formula I can comprise an alkylenyl spacer, such as a linear alkylenyl spacer. In some aspects, the alkylenyl spacer attached to the anchoring moiety AM and/or BAM is selected from the group consisting of C1, C2, C3, C4, C5, C6, C7, C8, C9, C10, C11, C12, C13, C14, or C15, wherein C denotes a divalent methylene unit (—CH₂—) and the numeral indicates the number of methylene units in the alkylenyl spacer. In some aspects, the alkylenyl spacer comprises a single methylenyl (C1; —CH₂—). In some aspects, the alkylenyl spacer is C2 (—CH₂CH₂—). In some aspects, the alkylenyl spacer is C3. In some aspects, the alkylenyl spacer is C4. In some aspects, the alkylenyl spacer is C5. In some aspects, the alkylenyl spacer is C6. In some aspects, the alkylenyl spacer is C7. In some aspects, the alkylenyl spacer is C8. In some aspects, the alkylenyl spacer is C9. In some aspects, the alkylenyl spacer is C10. In some aspects, the alkylenyl spacer is C11. In some aspects, the alkylenyl spacer is C12. In some aspects, the alkylenyl spacer is C13. In some aspects, the alkylenyl spacer is C14. In some aspects, the alkylenyl spacer is C15.

In some aspects, one or more spacers (e.g., SP₁, SP₂, SP₃, and/or SP₄) in Formula I independently comprise an alkylenyl spacer, a glycol-based spacer, or a combination thereof. In some aspects, the SPI optional first spacer comprises or consists of an alkylenyl spacer which is C1, C2, C3, C4, C5, C6, C7, C8, C9, C10, C11, C12, C13, C14, or C15 (e.g., propylene, butylene, hexylene, C2-C15 alkylene, C2-C10 alkylene, or C2-C6 alkylene). In some aspects, SPI is a C3 or C6 alkylenyl spacer. In some aspects, the SP₂ optional second spacer comprises or consists of an alkylenyl spacer which is C1, C2, C3, C4, C5, C6, C7, C8, C9, C10, C11, C12, C13, C14, or C15 (e.g., propylene, butylene, hexylene, C2-C15 alkylene, C2-C10 alkylene, or C2-C6 alkylene). In some aspects, SP₂ is a C3 or C6 alkylenyl spacer. In some aspects, the SP3 optional second spacer comprises or consists of an alkylenyl spacer which is C1, C2, C3, C4, C5, C6, C7, C8, C9, C10, C11, C12, C13, C14, or C15 (e.g., propylene, butylene, hexylene, C2-C15 alkylene, C2-C10 alkylene, or C2-C6 alkylene). In some aspects, SP3 is a C3 or C6 alkylenyl spacer. In some aspects, the SP4 optional second spacer comprises or consists of an alkylenyl spacer which is C1, C2, C3, C4, C5, C6, C7, C8, C9, C10, C11, C12, C13, C14, or C15 (e.g., propylene, butylene, hexylene, C2-C15 alkylene, C2-C10 alkylene, or C2-C6 alkylene). In some aspects, SP₄ is a C3 or C6 alkylenyl spacer.

In some aspects, the spacer comprises an substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkylheterocyclylalkyl, alkylheterocyclylalkenyl, alkenylheterocyclylalkenyl, or alkenylheterocyclylalkyl, alkynylheteroarylalkynyl, alkylheterocyclylalkynyl, alkenylheterocyclylalkynyl.

Optionally these spacers are substituted with one or more (e.g., 1, 2, 3, 4, 5, 6, or 7) substituents. Substituents include, for example, hydroxy, alkoxy (e.g., —O—(C₁₋₈ alkyl)), straight or branched chain alkyl (e.g., C₁₋₁₅ alkyl or C₁₋₈ alkyl), amino, aminoalkyl (e.g., C₁-C₁₅ alkylamino), phosphoramidito, phosphato, phosphoramidato, phosphorodithioato, thiophosphato, hydrazido, hydrazino, halo (e.g., F, Cl, Br, or I), aryl, —C(O)R′, —OC(O)R′, —C(O) OR′, —C(O)NH₂, —C(O)NHR′, —C(O)N(R′)₂—, NHC(O)R′, —S(O)₂R′, —S(O)R′, —NH(R′), —N(R′)₂, carboxylic acid halide, ether, sulfonyl halide, imidate ester, isocyanato, isothiocyanato, haloformate, carboduimide adduct, aldehyde, ketone, sulfhydryl, haloacetyl, alkyl halide, alkyl sulfonat, C(═O) CH═CHC(═O) (maleimide), thioether, cyano,nitro, sugar (e.g., mannosyl, galactosyl, and glucosyl), α,β-unsaturated carbonyl, alkyl mercurial, or α,β-unsaturated sulfonyl, where each R′ is independently H, C₁₋₈ alkyl, or aryl.

Examples of aliphatic linkers include the following structures: —O—CO—O—; —NH—CO—O—; —NH—CO—NH—; —NH—(CH₂)ₙ₁—; —S—(CH₂)ₙ₁—; —CO—(CH₂)ₙ₁—CO—; —CO—(CH₂)ₙ₁—NH—; —NH—(CH₂)ₙ₁—NH—; —CO—NH—(CH₂)ₙ₁—NH—CO—; —C(═S)—NH—(CH₂)ₙ₁—NH—CO—; —C(═S)—NH— (CH₂)ₙ₁—NH—C—(═S)—; —CO—O—(CH₂)ₙ₁—O—CO—; —C(═S)—O— (CH₂)ₙ₁—O—CO—; —C(═S)—O—(CH₂)ₙ₁—O—C—(—S)—; —CO—NH— (CH₂)ₙ₁—O—CO—; —C(═S)—NH— (CH₂)ₙ₁—O— CO—; —C(═S)—NH—(CH₂)ₙ—O—C—(═S)—; —CO—NH— (CH₂)ₙ-O—CO—; —C(═S)—NH—(CH₂)ₙ₁—CO—; —C(═S)—O—(CH₂)ₙ₁—NH—CO—; —C(═S)—NH—(CH₂)ₙ₁—O—C— (═S)—; —NH— (CH₂CH₂O) n2—CH (CH₂OH)—; —NH— (CH₂CH₂O)ₙ₂—CH₂—; —NH—(CH₂CH₂O)ₙ₂—CH₂—CO—; —O—(CH₂)ₙ₃—S—S—(CH₂)ₙ₄—O—P(═O)₂—; —CO— (CH₂)ₙ₃—O—CO—NH—(CH₂)ₙ₄—; —CO—(CH₂)ₙ₃—CO—NH—(CH₂)ₙ₄—; (CH₂)ₙ₁NH—; —C(O)(CH₂)ₙ₁ NH—; —C(O)—(CH₂)ₙ₁-C(O)—; —C(O)(CH₂)ₙ₁—C(O)O—; —C(O)—O—; —C(O)—(CH₂)ₙ₁—NH—C(O)—; —C(O)—(CH₂)ₙ₁—; —C(O)—NH—; —C(O)—; —(CH₂)ₙ₁—C(O)—; —(CH₂)ₙ₁—C(O)O—; —(CH₂)ₙ₁—; —(CH₂)ₙ₁—NH—C(O)—; wherein n1 is an integer between 1 and 15 (e.g., 2 to 15, or 2 to 12); n2 is an integer between 1 and 15 (e.g., 1 to 10, or 1 to 6); n3 and n4 can be the same or different and are each an integer between 1 and 15 (e.g., 1 to 10, or 1 to 6), and wherein the total number of carbon units in the linker is C₁₅ or less.

### Glycol-Based Spacer

In some aspects, a spacer (e.g., SP₁, SP₂, SP₃, and/or SP₄) in Formula I can comprise a glycol-based spacer. In some aspects, the glycol-based spacer comprises two or more—OCH₂CH₂-repeat units and can be formed from, e.g., diethylene glycol, triethylene glycol, tetraethylene glycol (TEG), pentaethylene glycol, hexaethylene glycol (HEG), heptaethylene glycol, octaethylene glycol, nonaethylene glycol, or decaethylene glycol to provide the corresponding number of —OCH₂CH₂— repeat units. In some aspects, the glycol-based spacer can comprise 11, 12, 13, 14, or 15 glycol-based repeat units (—OCH₂CH₂—). In some aspects, the glycol-based spacer has between 2 and 10, between 2 and 5, between 5 and 10, or between 10 and 15 glycol-based repeat units. In some aspects, the glycol-based spacer is formed from HEG (i.e., 6 glycol repeat units). In some aspects, the glycol-based spacer is formed from TEG (i.e., 4 glycol repeat units).

In some aspects, the glycol-based spacer can comprise a branched polyglycerol of the formula (R³—O—(CH₂—CHOR⁵—CH₂—O)ₙ—) with R⁵ being hydrogen or a linear glycerol chain of the formula (R³—O—(CH₂—CHOH-CH₂—O)ₙ—) and R³ being hydrogen, methyl or ethyl, and n is an integer of 1 to 15 (e.g., 1, 2, 3, 4, 5, 6, 7, 8, 9, 10, 11, 12, 13, 14, or 15). In some aspects, the glycol-based spacer can comprise a hyperbranched polyglycerol of the formula (R³—O—(CH₂—CHOR⁵—CH₂—O)ₙ—) with R⁵ being hydrogen or a glycerol chain described by the formula (R³—O—(CH₂—CHOR⁶—CH₂—O)ₙ—), with R⁶ being hydrogen or a glycerol chain of the formula (R³—O—(CH₂—CHOR⁷—CH₂—O)ₙ—), with R⁷ being hydrogen or a linear glycerol chain of the formula (R³—O—(CH₂—CHOH-CH₂—O)ₙ—) and R³ being hydrogen, methyl or ethyl. In each instance, n is an integer of 1 to 15 (e.g., 1, 2, 3, 4, 5, 6, 7, 8, 9, 10, 11, 12, 13, 14, or 15). Hyperbranched glycerol and methods of synthesis are described in, for example, Oudshorn et al. (Biomaterials, 2006, 27:5471-5479) and Wilms et al. (Acc. Chem. Res., 2010, 43, 129-41).

In some aspects, the SPI optional first spacer comprises or consists of a glycol-based spacer which has 2 (diethylene glycol), 3 (triethylene glycol), 4 (tetraethylene glycol; TEG), 5 (pentaethylene glycol), 6 (hexaethylene glycol; HEG), 7, 8, 9, 10, 11, 12, 13, 14, or 15 glycol units. In some aspects, SPI is a tetraethylene glycol (TEG) or hexaethylene glycol (HEG) glycol-based spacer. In some aspects, the SP₂ optional second spacer comprises or consists of a glycol-based spacer which has 2 (diethylene glycol), 3 (triethylene glycol), 4 (tetraethylene glycol; TEG), 5 (pentaethylene glycol), 6 (hexaethylene glycol; HEG), 7, 8, 9, 10, 11, 12, 13, 14, or 15 glycol-based units. In some aspects, SP₂ is a tetraethylene glycol (TEG) or hexaethylene glycol (HEG) glycol-based spacer. In some aspects, the SP₃ optional third spacer comprises or consists of a glycol-based spacer which has 2 (diethylene glycol), 3 (triethylene glycol), 4 (tetraethylene glycol; TEG), 5 (pentaethylene glycol), 6 (hexaethylene glycol; HEG), 7, 8, 9, 10, 11, 12, 13, 14, or 15 glycol units. In some aspects, SP₃ is a tetraethylene glycol (TEG) or hexaethylene glycol (HEG) glycol-based spacer. In some aspects, the SP₄ optional fourth spacer comprises or consists of a glycol-based spacer which has 2 (diethylene glycol), 3 (triethylene glycol), 4 (tetraethylene glycol; TEG), 5 (pentaethylene glycol), 6 (hexaethylene glycol; HEG), 7, 8, 9, 10, 11, 12, 13, 14, or 15 glycol units. In some aspects, SP₄ is a tetraethylene glycol (TEG) or hexaethylene glycol (HEG) glycol-based spacer.

In some aspects, the SPI and SP₂ spacers can comprise an alkylenyl spacer and the SP₃ spacer can comprise a glycol-based spacer. In some aspects, the SP₂ and SP₃ spacers can comprise an alkylenyl spacer and the SPI spacer can comprise a glycol-based spacer. In some aspects, the SP₂ and SP₃ spacers can comprise an alkylenyl spacer and the SPI and SP₄ spacers are absent.

In some aspects, each non-cleavable spacer is independently selected from the group consisting of alkyl (e.g., C2, C3, C4, C5, C6, C7 or C8), glycol (e.g., diethylene glycol, triethylene glycol, tetraethylene glycol (TEG), hexaethylene glycol (HEG), pentaethylene glycol, polyethylene glycol (PEG)), glycerol (e.g., diglycerol, triglycerol, tetraglycerol (TG), pentaglycerol, a hexaglycerol (HG), polyglycerol (PG)), succinimide, maleimide, or any combination thereof.

### Polyethylene Glycol Spacer

In some aspects, a spacer (e.g., SP₁, SP₂, SP₃, and/or SP₄) in Formula I or II can comprise or consist of a glycol-based spacer that can be considered a polyethylene glycol (PEG) residue of the formula —(O—CH₂—CH₂)ₙ— or —(O—CH₂—CH₂)ₙ—O—, wherein n is an integer between 1 and 200 (e.g., 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200). These PEG can be described as PEG₁, PEG₂, PEG₃, PEG₄, PEG₅, PEG₆, PEG₇, PEG₈, PEG₉, PEG₁₀, PEG₁₁, PEG₁₂, PEG₁₃, PEG₁₄, PEG₁₅, PEG₂₅, PEG₅₀, PEG_{75,} PEG₁₀₀, PEG₁₂₅, PEG₁₅₀, PEG₁₇₅, PEG₂₀₀, or PEGI-O—, PEG₂-O—, PEG₃-O—, PEG+—O—, PEG₅-O—, PEG₆-O—, PEG₇-O—, PEG₈-O—, PEG₉-O—, PEG₁₀-O—, PEG₁₁-O—, PEG₁₂-O—, PEG₁₃-O—, PEG₁₄-O—, PEG₁₅-O—, PEG₂₅-O—, PEG₅₀-O—, PEG₇₅-O—, PEG₁₀₀-O—, PEG₁₂₅-O—, PEG₁₅₀-O—, PEG₁₇₅-O—, or PEG₂₀₀-O—. Prior to conjugation to another moiety (e.g., AM or BAM) that forms the construct of Formula I or II, the PEG residue can be formed from R¹—(O—CH₂—CH₂)ₙ-R¹ or R¹—(O—CH₂—CH₂) n—OR1, wherein R¹ is hydrogen, methyl, or ethyl and n is an integer between 1 and 200 (e.g., 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200).

In some aspects, the PEG is a branched PEG. Branched PEGs have three to ten PEG chains emanating from a central core group. In certain aspects, the PEG moiety is a monodisperse polyethylene glycol. In the context of the present disclosure, a monodisperse polyethylene glycol (mdPEG) is a PEG that has a single, defined chain length and molecular weight.

In some aspects, the PEG is a star PEG. Star PEGs have about 10 to 15 PEG chains emanating from a central core group. In some aspects, the PEG is a comb PEGs. Comb PEGs have multiple PEG chains normally grafted onto a polymer backbone.

In some aspects, a spacer (e.g., SP₁, SP₂, SP₃, and/or SP₄) in Formula I can comprise or consist of a polyglycerol (PG) residue characterized by the formula —O—(CH₂—CHOH—CH₂O)ₙ—, wherein n is an integer between 1 and 15 (e.g., 1, 2, 3, 4, 5, 6, 7, 8, 9, 10, 11, 12, 13, 14 or 15). These polyglycerol residues can be described as PG₁, PG₂, PG₃, PG₄, PG₅, PG₆, PG₇, PG₈, PG₉, PG₁₀, PG₁₁, PG₁₂, PG₁₃, PG₁₄, or PG₁₅.

In some aspects, the anchoring moiety AM comprises or consists of a vitamin, as described herein, and an alkylenyl spacer. In some aspects, the anchoring moiety AM comprises or consists of tocopherol and an alkylenyl spacer. In some aspects, the anchoring moiety AM comprises or consists of a vitamin and an octyl (C8) alkylenyl spacer, such as tocopherol and an octyl (C8) alkylenyl spacer.

In some aspects, the anchoring moiety AM comprises or consists of a fatty acid and an alkylenyl spacer. In some aspects, the anchoring moiety AM comprises or consists of a palmitate and an alkylenyl spacer. In some aspects, the anchoring moiety AM comprises or consists of a fatty acid and a hexyl (C6) alkylenyl spacer, such as palmitate and a hexyl (C6) alkylenyl spacer. . . . In some aspects, the anchoring moiety AM comprises or consists of dipalmitoylphosphatidic acid and an alkylenyl spacer.

In some aspects, the anchoring moiety AM comprises or consists of a sterol and a glycol-based spacer. In some aspects, the anchoring moiety AM comprises or consists of cholesterol and a glycol-based spacer. In some aspects, the anchoring moiety AM comprises or consists of a sterol and a TEG glycol-based spacer, such as cholesterol and a TEG glycol-based spacer. In some aspects, the anchoring moiety AM comprises or consists of a sterol and an alkylenyl spacer. In some aspects, the anchoring moiety AM comprises or consists of a sterol and a hexyl (C6) alkylenyl spacer. In some aspects, the anchoring moiety AM comprises or consists of cholesterol and an alkylenyl spacer, such as cholesterol and a hexyl (C6) alkylenyl spacer.

### Non-cleavable Linkers

In some aspects, the linker combination comprises a “non-cleavable linkage,” which is a linker and/or spacer that that includes chemical bonds that are substantially resistant to cleavage. Non-cleavable linkers are substantially resistant to, for example, acid-induced cleavage, photo-induced cleavage, peptidase-induced cleavage, esterase-induced cleavage, and disulfide bond cleavage, particularly at conditions under which a cyclic dinucleotide and/or the antibody does not lose its activity. Non-cleavable linkers are any chemical moiety capable of linking two or more components of a construct disclosed herein, e.g., a construct of Formula I or II. In particular, the non-cleavable linker can link any two parts of the construct, e.g., the anchoring moiety AM and/or a spacer SP and/or the biologically active molecule BAM. Typically, the non-cleavable linker is part of a spacer (e.g., SP₁, SP₂, SP₃, and/or SP₄), as described herein, but in some aspsects, the AM or BAM is modified to include the non-cleavable linkage.

In some aspects, the linker combination comprises a non-cleavable linker comprising, e.g., tetraethylene glycol (TEG), hexaethylene glycol (HEG), polyethylene glycol (PEG), glycerol, C2 to C12 alkyl, succinimidyl, or any combination thereof. In some aspects, the non-cleavable linkage comprises a spacer, as described herein, to link the AM and/or BAM to the non-cleavable linkage.

### Biologically Active Molecule

The biologically active molecule BAM is an agent that acts on a target (e.g., a target cell). Contacting can occur in vitro or in a subject. Non-limiting examples of biologically active molecules BAM that can attached to an EV (e.g., exosome) as described in the present disclosure include agents such as polynucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, mRNA, siRNA, shRNA, or an antisense oligonucleotide (ASO)), amino acids (e.g., amino acids comprising a detectable moiety or a toxin that disrupt translation), polypeptides (e.g., enzymes), lipids, carbohydrates, and small molecules (e.g., small molecule drugs and toxins), an antimirs, an RNA decoy, or any combination thereof. In some aspects, the BAM comprises a peptide, a polypeptidyl, a polynucleotidyl, a protein, an antibody or an antigen binding fragment thereof, a chemical compound, or any combination thereof. In some aspects, the BAM comprises an antisense oligonucleotidyl (ASO), siRNA, miRNA, shRNA, a nucleic acid, or any combination thereof.

In some aspects, an EV (e.g., exosome) disclosed herein can comprise more than one biologically active molecule BAM attached, e.g., using the constructs disclosed herein. For example, in some aspects, the EV (e.g., exosome) can comprise multiple populations of constructs of the present disclosure (e.g., a construct of Formula I or II), wherein each population of constructs carry a different biologically active molecule BAM.

Accordingly, in some aspects a population of EVs, e.g., exosomes, of the present disclosed can comprise a plurality of construct as exemplified below.

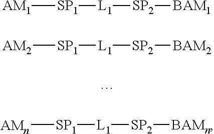

wherein [AM₁] . . . [AMₙ] can be the same or different anchoring moieties, each SP₁, L₁, and SP₂ can be the same or different, and [BAM₁]. [BAMₙ] can be the same of different biologically active molecules.

In some aspects, the biologically active molecule BAM comprises or consists of a peptide, a protein, an antibody or an antigen binding portion thereof, or any combination thereof. In some aspects, the antigen binding portion thereof comprises scFv, (scFv) 2, Fab, Fab′, F(ab′)₂, F(ab1)2, Fv, dAb, and Fd fragment, diabodys, antibody-related polypeptide, or any fragment thereof. In some aspects, the antibody or antigen binding portion thereof can bind to a Protein X protein present in the membrane of the EV (e.g., exosome).

In some aspects, the biologically active molecule BAM targets a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L₁), programmed death ligand 2 (PD-L₂), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, or combinations thereof.

In some aspects, the biologically active molecule BAM is a targeting moiety, e.g., an antibody or binding portion thereof or a ligand that specifically binds to a marker on a muscle cell. In some aspects, the muscle cell is a smooth muscle cell. In some aspects, the muscle cell is a skeletal muscle cell. In some aspects, the muscle cell is a cardiac muscle cell. In some aspects, the marker on the muscle cell is selected from alpha-smooth muscle actin, VE-cadherin, caldesmon/CALDI, calponin 1, hexim 1, histamine H2 R; motilin R/GPR38, transgelin/TAGLN, and any combination thereof. In some aspects, the marker on the muscle cell is selected from alpha-sarcoglycan, beta-sarcoglycan, calpain inhibitors, creatine kinase MM/CKMM, eIF5A, enolase 2/neuron-specific enolase, epsilon-sarcoglycan, FABP3/H-FABP, GDF-8/Myostatin, GDF-11/GDF-8, integrin alpha 7, integrin alpha 7 beta 1, integrin beta 1/CD29, MCAM/CD146, MyoD, myogenin, myosin light chain kinase inhibitors, NCAM-1/CD56, troponin I, and any combination thereof. In some aspects, the marker on the muscle cell is myosin heavy chain, myosin light chain, or a combination thereof.

In some aspects, the biologically active molecule BAM is a small molecule. In some aspects, the small molecule is a proteolysis-targeting chimera (PROTAC). In some aspects, the biologically active molecule BAM is a small molecule comprising a synthetic antineoplastic agent (e.g., monomethyl auristatin E (MMAE)(vedotin)), a cytokine release inhibitor (e.g., MCC950), an mTOR inhibitor (e.g., rapamycin and its analogs (rapalogs)), an autotaxin inhibitor (e.g., PAT409 or PAT505), a lysophosphatidic acid receptor agonist (e.g., BMS—986020), a STING antagonist (e.g., CL656), or any combination thereof. In some aspects, a biologically active molecule BAM comprises a morpholino backbone structure as disclosed in U.S. Pat. No. 5,034,506, which is herein incorporated by reference in its entirety.).

In some aspects, the biologically active molecule BAM comprises a nucleotide, wherein the nucleotide is a stimulator of interferon genes protein (STING) agonist. STING is a cytosolic sensor of cyclic dinucleotides that is typically produced by bacteria. Upon activation, it leads to the production of type I interferons and initiates an immune response

In some aspects, the STING agonist comprises a cyclic nucleotide STING agonist or a non-cyclic dinucleotide STING agonist. Cyclic purine dinucleotides (CDN) such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3′5′, bonds linking the cyclic dinucleotides, or any combination thereof. Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications. Non-cyclic dinucleotide agonists can also be used, such as 5,6-dimethylxanthenone-4-acetic acid (DMXAA), or any other non-cyclic dinucleotide agonist known in the art.

It is contemplated that any STING agonist can be used as the biologically active molecule BAM. STING agonists include DMXAA, STING agonist-1, ML RR-S2 CDA, ML RR-S2c-di-GMP, ML-RR-S2 cGAMP, 2′3′-c-di-AM (PS) 2, 2′3′-cGAMP, 2′3′-cGAMPdFHS, 3′3′-cGAMP, 3′3′-cGAMPdFSH, cAIMP, cAIM (PS) 2, 3′3′-cAIMP, 3′3′-cAIMPdFSH, 2′2′-cGAMP, 2′3′-cGAM (PS) 2, 3′3′-cGAMP, c-di-AMP, 2′3′-c-di-AMP, 2′3′-c-di-AM (PS) 2, c-di-GMP, 2′3′-c-di-GMP, c-di-IMP, c-di-UMP or any combination thereof. In a specific aspect, the STING agonist is 3′3′-cAIMPdFSH, alternatively named 3-3 cAIMPdFSH. Additional STING agonists known in the art can also be used.

In some aspects, the biologically active molecule BAM is an antibody or antigen binding fragment thereof. In some aspects, the biologically active molecule BAM is an antibody-drug conjugate (ADC). In some aspects, the biologically active molecule BAM is a fusogenic peptide.

In some aspects, the biologically active molecule BAM targets macrophages. In other aspects, the biologically active molecule induces macrophage polarization. Macrophage polarization is a process by which macrophages adopt different functional programs in response to the signals from their microenvironment. This ability is connected to their multiple roles in the organism: they are powerful effector cells of innate immune system, but also important in removal of cellular debris, embryonic development and tissue repair.

By simplified classification, macrophage phenotype has been divided into 2 groups: Ml (classically activated macrophages) and M2 (alternatively activated macrophages). This broad classification was based on in vitro studies, in which cultured macrophages were treated with molecules that stimulated their phenotype switching to particular state. In addition to chemical stimulation, it has been shown that the stiffness of the underlying substrate a macrophage is grown on can direct polarization state, functional roles and migration mode. MI macrophages were described as the pro-inflammatory type, important in direct host-defense against pathogens, such as phagocytosis and secretion of pro-inflammatory cytokines and microbicidal molecules. M2 macrophages were described to have quite the opposite function: regulation of the resolution phase of inflammation and the repair of damaged tissues. Later, more extensive in vitro and ex vivo studies have shown that macrophage phenotypes are much more diverse, overlapping with each other in terms of gene expression and function, revealing that these many hybrid states form a continuum of activation states which depend on the microenvironment. Moreover, in vivo, there is a high diversity in gene expression profile between different populations of tissue macrophages. Macrophage activation spectrum is thus considered to be wider, involving complex regulatory pathway to response to plethora of different signals from the environment. The diversity of macrophage phenotypes still remains to be fully characterized in vivo.

The imbalance of the macrophage types is related to a number of immunity-related diseases. For example, increased M1/M2 ratio may correlate with development of inflammatory bowel disease, as well as obesity in mice. On the other side, in vitro experiments implicated M2 macrophages as the primary mediators of tissue fibrosis. Several studies have associated the fibrotic profile of M2 macrophages with the pathogenesis of systemic sclerosis. Non-limiting examples of the macrophage targeting biologically active molecules are: PI3KY (phosphatidylinositol-4,5-bisphosphate 3-kinase catalytic subunit gamma), RIPI (Receptor Interacting Protein (RIP) kinase 1, RIPK1), HIF-1α (Hypoxia-inducible factor 1-alpha), AHR1 (Adhesion and hyphal regulator 1), miR146a, miR155, IRF4 (Interferon regulatory factor 4), PPARγ (Peroxisome proliferator -activated receptor gamma), IL-4RA (Interleukin-4 receptor subunit alpha), TLR8 (Toll-like receptor 8), and TGF-β1 (Transforming growth factor beta-1 proprotein)

In some aspects, the biologically active molecule BAM comprises or consists of an antisense oligonucleotide (ASO). In some aspects, the ASO is a gapmer, a mixmer, or a totalmer. In some aspects, the ASO can target pre-mRNA or a mature mRNA, including protein coding regions (exons), non coding regions (e.g., 5′ or 3′ unstranslated regions, or introns), intron-exon junctions, or regulatory regions (e.g., promoters). In some aspects, the ASO targets a protein transcript, e.g., a STAT6 transcript, an EGFP transcript, a CEBP/β transcript, a STAT3 transcript, a KRAS transcript, an NRAS transcript, an NLPR3 transcript, an FFLUC transcript, an RLUC transcript, a MYC transcript, or any combination thereof.

In some aspects, the ASO can comprise one or more nucleosides which have a modified sugar moiety, i.e., a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those in which the ribose ring structure has been modified, e.g., by replacement with a hexose ring (HNA), a bicyclic ring, which typically has a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in RNA nucleosides. Substituents can, for example, be introduced at the 2′, 3′, 4′, and/or 5′ positions. Nucleosides with modified sugar moieties also include 2′ modified nucleosides, such as 2′ substituted nucleosides. A 2′ sugar modified nucleoside is a nucleoside which has a substituent other than H or -OH at the 2′ position (2′ substituted nucleoside) or comprises a 2′ linked biradical, and includes 2′ substituted nucleosides and LNA (2′-4′ biradical bridged) nucleosides. For example, the 2′ modified sugar can provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′—O-methoxyethyl-RNA (MOE), 2′-amino-DNA, 2′-fluoro-RNA, 2′-fluoro-DNA, arabino nucleic acids (ANA), and 2′-fluoro-ANA nucleoside. Further examples are provided in Freier & Altmann; Nucl. Acid Res., 1997, 25, 4429-4443; Uhlmann, Curr. Opinion in Drug Development, 2000, 3 (2), 293-213; and Deleavey and Damha, Chemistry and Biology 2012, 19, 937. Below are illustrations of some 2′ substituted modified nucleosides.

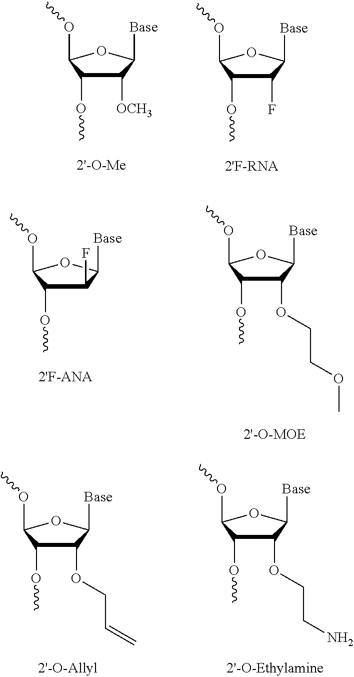

LNA nucleosides are modified nucleosides which comprise a linker group (referred to as a biradical or a bridge) between C₂′ and C₄′ of the ribose sugar ring of a nucleoside (i.e., 2′-4′ bridge), which restricts or locks the conformation of the ribose ring. These nucleosides are also termed bridged nucleic acid or bicyclic nucleic acid (BNA). The locking of the conformation of the ribose is associated with an enhanced affinity of hybridization (duplex stabilization) when the LNA is incorporated into an oligonucleotide for a complementary RNA or DNA molecule. This can e routinely determined by measuring the melting temperature of the oligonucleotide/complement duplex.

Non limiting, exemplary LNA nucleosides are disclosed in WO99/014226, WO00/66604, WO98/039352, WO2004/046160, WO00/047599, WO2007/134181, WO2010/077578, WO2010/036698, WO2007/090071, WO2009/006478, WO2011/156202, WO2008/154401, WO2009/067647, WO2008/150729, Morita et al., Bioorganic & Med. Chem. Lett., 12, 73-76, Seth et al., J. Org. Chem., 2010, Vol 75 (5) pp. 1569-81, or Mitsuoka et al., Nucleic Acids Research, 2009, 37 (4), 1225-1238, all of which are herein incorporated by reference in their entireties. In some aspects, the modified nucleoside or the LNA nucleosides of an ASO of the disclosure has a general structure of the Formula X or Formula XI:

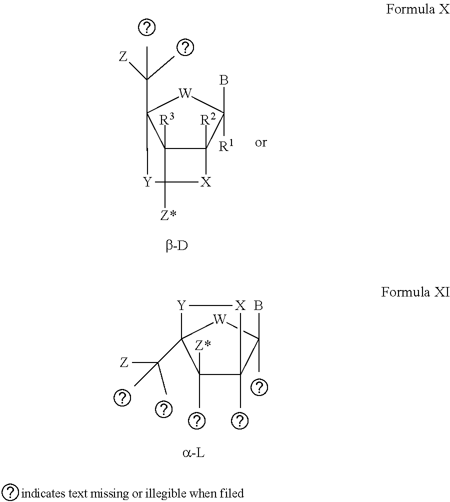

wherein
B is a nucleobase or a modified nucleobase moiety;
W is selected from —O—, —S—, —N(R^{a})—, —C(R^{a}R^{b})—, in particular —O—;
X is O;
Y is CH₂;
Z is an internucleoside linkage to an adjacent nucleoside or a 5′-terminal group;
Z* is an internucleoside linkage to an adjacent nucleoside or a 3′-terminal group;
R¹, R², R³, R⁵ and R⁵* are independently selected from hydrogen, halogen, alkyl, alkenyl, alkynyl, hydroxy, alkoxy, alkoxyalkyl, alkenyloxy, carboxyl, alkoxycarbonyl, alkylcarbonyl, formyl, azide, heterocyclyl, and aryl; and
R^{a} and R^{b} are independently selected from hydrogen and alkyl.

### ASO Targeting NLRP3

In some aspects, the biologically active molecule BAM is an anti-NLRP3 ASO. NLRP3 (NLRP3) is also known as NLR family pyrin domain containing 3. Unless indicated otherwise, the term “NLRP3,” as used herein, can refer to NLRP3 from one or more species (e.g., humans, non-human primates, dogs, cats, guinea pigs, rabbits, rats, mice, horses, cattle, and bears). The sequence for the human NLRP3 gene can be found under publicly available GenBank Accession Number NC_000001.11:247416156-247449108. The human NLRP3 gene is found at chromosome location 1q44 at 247,416,156-247,449,108. The sequence for the human NLRP 3 pre-mRNA transcript (SEQ ID NO: 1) corresponds to the reverse complement of residues 247,416,156-247,449,108 of chromosome 1q44. The NLRP3 mRNA sequence (GenBank Accession No.

NM_001079821.2) is provided in SEQ ID NO: 3, except that the nucleotide “t” in SEQ ID NO: 3 is shown as “u” in the mRNA. The sequence for human NLRP3 protein can be found under publicly available Accession Numbers: Q96P20, (canonical sequence, SEQ ID NO: 2), Q96P20-2 (SEQ ID NO: 4), Q96P20-3 (SEQ ID NO: 5), Q96P20-4 (SEQ ID NO: 6), Q96P20-5 (SEQ ID NO: 7), and Q96P20-6 (SEQ ID NO: 8), each of which is incorporated by reference herein in its entirety. The anti-NLRP3 ASOs of the present disclosure can be designed to reduce or inhibit expression of the natural variants of the NLRP3 protein.

An example of a target nucleic acid sequence of the anti-NLRP3 ASOs is NLRP3 pre-mRNA. SEQ ID NO: 1 represents a human NLRP3 genomic sequence (i.e., reverse complement of nucleotides 247,416,156-247,449,108 of chromosome 1q44). SEQ ID NO: 1 is identical to a NLRP3 pre-mRNA sequence except that nucleotide “t” in SEQ ID NO: 1 is shown as “u” in pre-mRNA. In certain aspects, the “target nucleic acid” comprises an intron of a NLRP3 protein-encoding nucleic acids or naturally occurring variants thereof, and RNA nucleic acids derived therefrom, e.g., pre-mRNA. In other aspects, the target nucleic acid comprises an exon region of a NLRP3 protein-encoding nucleic acids or naturally occurring variants thereof, and RNA nucleic acids derived therefrom, e.g., pre-mRNA. In yet other aspects, the target nucleic acid comprises an exon-intron junction of a NLRP3 protein-encoding nucleic acids or naturally occurring variants thereof, and RNA nucleic acids derived therefrom, e.g., pre-mRNA. The human NLRP3 protein sequence encoded by the NLRP3 pre-mRNA is shown as SEQ ID NO: 3. In other aspects, the target nucleic acid comprises an untranslated region of a NLRP3 protein-encoding nucleic acids or naturally occurring variants thereof, e.g., 5′ UTR, 3′ UTR, or both.

In some aspects, an anti-NLRP3 ASO of the disclosure hybridizes to a region within the introns of a NLRP3 transcript, e.g., SEQ ID NO: 1. In certain aspects, an anti-NLRP3 ASO of the disclosure hybridizes to a region within the exons of a NLRP3 transcript, e.g., SEQ ID NO: 1. In other aspects, an anti-NLRP3 ASO of the disclosure hybridizes to a region within the exon-intron junction of a NLRP3 transcript, e.g., SEQ ID NO: 1. In some aspects, an anti-NLRP3 ASO of the disclosure hybridizes to a region within a NLRP3 transcript (e.g., an intron, exon, or exon-intron junction), e.g., SEQ ID NO: 1, wherein the anti-NLRP3 ASO has a gapmer design.

In some aspects, the anti-NI.RP3 ASO targets an mRNA encoding a particular isoform of NLRP3 protein (e.g., Isoform 1). In some aspects, the anti-NLRP3 ASO targets all isoforms of NLRP3 protein. In other aspects, the anti-NLRP3 ASO targets two isoforms (e.g., Isoform 1 and Isoform 2, Isoform 3 and Isoform 4, and Isoform 5 and Isoform 6) of NLRP3 protein.

In some aspects, the nucleotide sequence of the anti-NLRP3 ASOs of the disclosure or the contiguous nucleotide sequence has at least 80% sequence identity to a sequence selected from SEQ ID NOs: 101 to 200, such as at least 80%, at least 85%, at least 90%, at least 91%, at least 92%, at least 93%, at least 94%, at least 95%, at least 96% sequence identity, at least 97% sequence identity, at least 98% sequence identity, at least 99% sequence identity, such as about 100% sequence identity (homologous).

In some aspects, the anti-NI.RP3 ASO (or contiguous nucleotide portion thereof) is selected from, or comprises, one of the sequences selected from the group consisting of SEQ ID NOs: 101 to 200 or a region of at least 10 contiguous nucleotides thereof, wherein the anti-NLRP3 ASO (or contiguous nucleotide portion thereof) can optionally comprise one, two, three, or four mismatches when compared to the corresponding NLRP3 transcript.

In some aspects, the anti-NLRP3 ASO comprises a sequence selected from the group consisting of SEQ ID NO: 101-200.

In some aspects, the anti-NLRP3 ASO comprises a sequence as set forth in any one of SEQ ID NOs: 101 to 200. In some aspects, the anti-NLRP3 ASO comprises or consists of a sequence at least 60%, at least 65%, at least 70%, at least 75%, at least 80%, at least 85%, at least 90%, at least 95%, at least 96%, at least 97%, at least 98%, at least 99%, or about 100% identical to a sequence set forth in SEQ ID NOs: 101 to 200. In some aspects, the anti-NLRP3 ASO (or contiguous nucleotide portion thereof) is selected from, or comprises, one of the sequences selected from the group consisting of SEQ ID NOs: 101 to 200 or a region of at least 8, 9, 10, 11, 12, 13, 14, 15, 16, 17, 18, 19, or 20 contiguous nucleotides thereof. In some aspects, the anti-NLRP3 ASO (or contiguous nucleotide portion thereof) is selected from, or comprises, one of the sequences selected from the group consisting of SEQ ID NOs: 101 to 200 or a region of at least 8, 9, 10, 11, 12, 13, 14, 15, 16, 17, 18, 19, or 20 contiguous nucleotides thereof, wherein the anti-NLRP3 ASO (or contiguous nucleotide portion thereof) can optionally comprise one, two, three, or four mismatches when compared to the corresponding NI.RP3 transcript. In some aspects, the anti-NLRP3 ASO (or contiguous nucleotide portion thereof) is selected from, or comprises, one of the sequences selected from the group consisting of SEQ ID NOs: 101 to 200 except for 1, 2, 3, 4, 5, 6, 7, 8, 9 or 10 substitutions, wherein the substituted ASO can bind to the NLRP3 transcript. In some aspects, the anti-NLRP3 ASO (or contiguous nucleotide portion thereof) is selected from, or comprises, one of the sequences selected from the group consisting of SEQ ID NOs: 101 to 200 or a region of at least 8, 9, 10, 11, 12, 13, 14, 15, 16, 17, 18, 19, or 20 contiguous nucleotides thereof, wherein the anti-NLRP3 ASO (or contiguous nucleotide portion thereof) can optionally comprise one, two, three, or four additional 5′ and/or 3′ nucleotides complementary to the corresponding NLRP3 transcript.

In some aspects, binding of an anti-NLRP3 ASO targeting a NLRP3 transcript disclosed herein to an mRNA transcript encoding NLRP3 can reduce expression levels and/or activity levels of NLRP3.

In some aspects, any anti-NLRP3 ASO described herein can be part of an EV (e.g., exosome) of the present disclosure, i.e., an EV (e.g., exosome) a comprising construct comprising a cleavable linker disclosed herein, e.g., a construct of Formula I or II in which the biologically active moiety BAM is an anti-NLRP3 ASO described herein or a combination thereof.

### ASO Targeting STAT6

In some aspects, the biologically active molecule BAM is an anti-STAT6 ASO. STAT6 (STAT6) is also known as signal transducer and activator of transcription 6. Unless indicated otherwise, the term “STAT6,” as used herein, can refer to STAT6 from one or more species (e.g., humans, non-human primates, dogs, cats, guinea pigs, rabbits, rats, mice, horses, cattle, and bears).

The sequence for the human STAT6 pre-mRNA transcript (SEQ ID NO: 11) corresponds to the reverse complement of residues 57111413-57095404, complement, of chromosome 12q13.3. The STAT6 mRNA sequence (GenBank Accession No. NM_001178078.1) is provided in SEQ ID NO: 13, except that the nucleotide “t” in SEQ ID NO: 13 is shown as “u” in the mRNA. The sequence for human STAT6 protein can be found under publicly available Accession Numbers: P42226-1, (canonical sequence, SEQ ID NO: 12), P42226-2 (SEQ ID NO: 14), and P42226-3 (SEQ ID NO: 15), each of which is incorporated by reference herein in its entirety.

Natural variants of the human STAT6 gene product are known. For example, natural variants of human STAT6 protein can contain one or more amino acid substitutions selected from: M118R, D₄₁₉N, and any combination thereof. Additional variants of human STAT6 protein resulting from alternative splicing are also known in the art. STAT6 Isoform 2 (identifier: P42226-2 at UniProt) differs from the canonical sequence (SEQ ID NO: 13) as follows: deletion of residues 1-174 and substitution of 175PSE177 with 175MEQ177 relative to SEQ ID NO: 13. The sequence of STAT6 Isoform 3 (identifier: P42226-3) differs from the canonical sequence (SEQ ID NO: 13) as follows: deletion of residues 1-110 relative to SEQ ID NO: 13. Therefore, the anti-STAT6 ASOs of the present disclosure can be designed to reduce or inhibit expression of the natural variants of the STAT6 protein.

An example of a target nucleic acid sequence of the anti-STAT6 ASOs is STAT6 pre-mRNA. SEQ ID NO: 11 represents a human STAT6 genomic sequence (i.e., reverse complement of nucleotides 57111413-57095404, complement, of chromosome 12q13.3). SEQ ID NO: 11 is identical to a STAT6 pre-mRNA sequence except that nucleotide “t” in SEQ ID NO: 11 is shown as “u” in pre-mRNA. In certain aspects, the “target nucleic acid” comprises an intron of a STAT6 protein-encoding nucleic acids or naturally occurring variants thereof, and RNA nucleic acids derived therefrom, e.g., pre-mRNA. In other aspects, the target nucleic acid comprises an exon region of a STAT6 protein-encoding nucleic acids or naturally occurring variants thereof, and RNA nucleic acids derived therefrom, e.g., pre-mRNA. In yet other aspects, the target nucleic acid comprises an exon-intron junction of a STAT6 protein-encoding nucleic acids or naturally occurring variants thereof, and RNA nucleic acids derived therefrom, e.g., pre-mRNA. The human STAT6 protein sequence encoded by the STAT6 pre-mRNA is shown as SEQ ID NO: 13. In other aspects, the target nucleic acid comprises an untranslated region of a STAT6 protein-encoding nucleic acids or naturally occurring variants thereof, e.g., 5′ UTR, 3′ UTR, or both.

In some aspects, an anti-STAT6 ASO of the disclosure hybridizes to a region within the introns of a STAT6 transcript, e.g., SEQ ID NO: 11. In certain aspects, an anti-STAT6 ASO of the disclosure hybridizes to a region within the exons of a STAT6 transcript, e.g., SEQ ID NO: 11. In other aspects, an anti-STAT6 ASO of the disclosure hybridizes to a region within the exon-intron junction of a STAT6 transcript, e.g., SEQ ID NO: 11. In some aspects, an anti-STAT6 ASO of the disclosure hybridizes to a region within a STAT6 transcript (e.g., an intron, exon, or exon-intron junction), e.g., SEQ ID NO: 11, wherein the anti-STAT6 ASO has a gapmer design.

In some aspects, the anti-STAT6 ASO targets an mRNA encoding a particular isoform of STAT6 protein (e.g., Isoform 1). In some aspects, the ASO targets all isoforms of STAT6 protein. In other aspects, the anti-STAT6 ASO targets two isoforms (e.g., Isoform 1 and Isoform 2, Isoform 1 and Isoform 3, or Isoform 2 and Isoform 3) of STAT6 protein.

In some aspects, a payload of the disclosure (e.g., ASO) hybridizes to a region within the introns of a STAT6 transcript. In certain aspects, the payload hybridizes to a region within the exons of a STAT6 transcript. In some aspects, the payload hybridizes to a region within the exon-intron junction of a STAT6 transcript. In some aspects, the payload hybridizes to a region within a STAT6 transcript (e.g., an intron, exon, or exon-intron junction). A non-limiting example of a payload (e.g., ASO) that can specifically target a region of a STAT6 transcript.

In some aspects, binding of an anti-STAT6 ASO targeting a STAT6 transcript disclosed herein to an mRNA transcript encoding STAT6 can reduce expression levels and/or activity levels of STAT6.

In some aspects, any anti-STAT6 ASO described herein can be part of an EV (e.g., exosome) of the present disclosure, i.e., an EV (e.g., exosome) a comprising construct comprising a cleavable linker disclosed herein, e.g., a construct of Formula I or II in which the biologically active moiety BAM is an anti-STAT6 ASO described herein or a combination thereof.

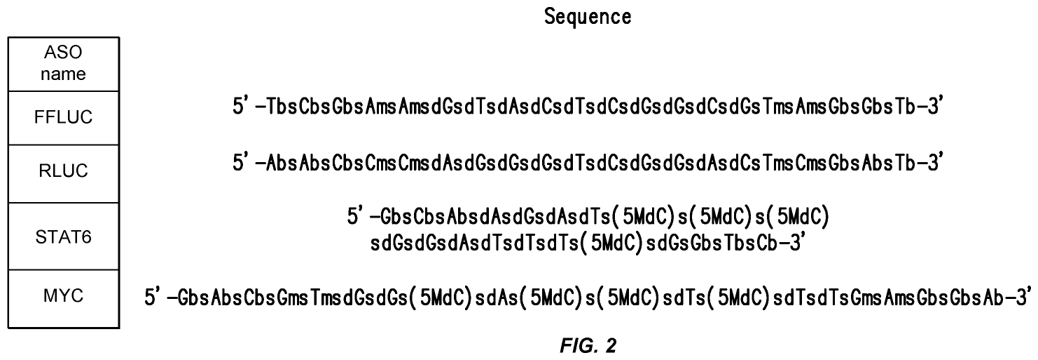

In some aspects, an anti-STAT6 ASO of the present disclosure comprises the base sequence of SEQ ID NO: 1091. In some aspects, an anti-STAT6 ASO of the present disclosure comprises the STAT 6 ASO sequence shown in FIG. 2.

### ASO Targeting MYC

In some aspects, the biologically active molecule BAM is an anti MYCASO. Unless indicated otherwise, the term “MYC,” as used herein, can refer to MYC from one or more species (e.g., humans, non-human primates, dogs, cats, guinea pigs, rabbits, rats, mice, horses, cattle, and bears).

In some aspects, an anti-MYC ASO of the present disclosure comprises the base sequence of SEQ ID NO: 1092. In some aspects, and anti-MYC ASO of the present disclosure comprises the MYC ASO sequence shown in FIG. 2.

### ASO Targeting CEBP/β

In some aspects, the biologically active molecule BAM is an anti-CEBP β ASO. Unless indicated otherwise, the term “CEBP/B,” as used herein, can refer to CEBP/β from one or more species (e.g., humans, non-human primates, dogs, cats, guinea pigs, rabbits, rats, mice, horses, cattle, and bears).

The sequence for the human CEBP β gene can be found under publicly available GenBank Accession Number NC_000020.11 (50190583 . . . 50192690). The human CEBP β gene is found at chromosome location 20q13.13 at 50190583-50192690.

The sequence for the human CEBP β pre-mRNA transcript (SEQ ID NO: 21) corresponds to the reverse complement of residues 50190583-50192690 of chromosome 20q13.13. The CEBP β mRNA sequence (GenBank Accession No. NM_001285878.1) is provided in SEQ ID NO: 23, except that the nucleotide “t” in SEQ ID NO: 23 is shown as “u” in the mRNA. The sequence for human CEBP/β protein can be found under publicly available Accession Numbers: P17676, (canonical sequence, SEQ ID NO: 22), P17676-2 (SEQ ID NO: 24), and P17676-3 (SEQ ID NO: 25), each of which is incorporated by reference herein in its entirety.

Natural variants of the human CEBP β gene product are known. For example, natural variants of human CEBP/β protein can contain one or more amino acid substitutions selected from: A241P, A253G, G195S, and any combination thereof. Additional variants of human CEBP/β protein resulting from alternative splicing are also known in the art. CEBP/β Isoform 2 (identifier: P17676-2 at UniProt) differs from the canonical sequence (SEQ ID NO: 23) as follows: deletion of residues 1-23 relative to SEQ ID NO: 23. The sequence of CEBP/β Isoform 3 (identifier: P17676-3) differs from the canonical sequence (SEQ ID NO: 23) as follows: deletion of residues 1-198 relative to SEQ ID NO: 23. Therefore, the anti-CEBPb ASOs of the present disclosure can be designed to reduce or inhibit expression of the natural variants of the protein.

An example of a target nucleic acid sequence of the anti-CEBPb ASOs is CEBP β pre-mRNA. SEQ ID NO: 21 represents a human CEBP β genomic sequence (i.e., reverse complement of nucleotides 50190583-50192690 of chromosome 20q13.13). SEQ ID NO: 21 is identical to a CEBP (pre-mRNA sequence except that nucleotide “t” in SEQ ID NO: 21 is shown as “u” in pre-mRNA. In certain aspects, the “target nucleic acid” comprises an intron of a CEBP/β protein-encoding nucleic acids or naturally occurring variants thereof, and RNA nucleic acids derived therefrom, e.g., pre-mRNA. In other aspects, the target nucleic acid comprises an exon region of a CEBP/β protein-encoding nucleic acids or naturally occurring variants thereof, and RNA nucleic acids derived therefrom, e.g., pre-mRNA. In yet other aspects, the target nucleic acid comprises an exon-intron junction of a CEBP/β protein-encoding nucleic acids or naturally occurring variants thereof, and RNA nucleic acids derived therefrom, e.g., pre-mRNA. The human CEBP/β protein sequence encoded by the CEBP β pre-mRNA is shown as SEQ ID NO: 23. In other aspects, the target nucleic acid comprises an untranslated region of a CEBP/β protein-encoding nucleic acids or naturally occurring variants thereof, e.g., 5′ UTR, 3′ UTR, or both.

In some aspects, an anti-CEBPb ASO of the disclosure hybridizes to a region within the introns of a CEBP β transcript, e.g., SEQ ID NO: 21. In certain aspects, an anti-CEBPb ASO of the disclosure hybridizes to a region within the exons of a CEBP β transcript, e.g., SEQ ID NO: 21. In other aspects, an anti-CEBPb ASO of the disclosure hybridizes to a region within the exon-intron junction of a CEBP β transcript, e.g., SEQ ID NO: 21. In some aspects, an anti-CEBPb ASO of the disclosure hybridizes to a region within a CEBP β transcript (e.g., an intron, exon, or exon-intron junction), e.g., SEQ ID NO: 21, wherein the anti-CEBPb ASO has a gapmer design.

In some aspects, the anti-CEBPb ASO targets an mRNA encoding a particular isoform of CEBP/β protein (e.g., Isoform 1). In some aspects, the anti-CEBPb ASO targets all isoforms of CEBP/β protein. In other aspects, the anti-CEBPb ASO targets two isoforms (e.g., Isoform 1 and Isoform 2, Isoform 1 and Isoform 3, or Isoform 2 and Isoform 3) of CEBP/β protein.

In some aspects, binding of an anti-CEBPb ASO targeting a CEBPb transcript disclosed herein to an mRNA transcript encoding (EBPb can reduce expression levels and/or activity levels of CEBPb.

In some aspects, any anti-(EBPb ASO described herein can be part of an EV (e.g., exosome) of the present disclosure, i.e., an EV (e.g., exosome) a comprising construct comprising a cleavable linker disclosed herein, e.g., a construct of Formula I or II in which the biologically active moiety BAM is an anti-CEBPb ASO described herein or a combination thereof.

### ASO Targeting STAT3

In some aspects, the biologically active molecule BAM is an anti-STAT3 ASO. Signal Transducer and Activator of Transcription 3 (STAT3) is a signal transducer and activator of transcription that transmits signals from cell surface receptors to the nucleus. STAT3 is frequently hyperactivated in many human cancers.

Signal transducer and activator of transcription 3 (STAT3) is known in the art by various names. Such names include: DNA-binding protein APRF, and acute-phase response factor. The mRNA encoding human STAT3 can be found at Genbank Accession Number NM_003150.3, and is represented by the sequence (SEQ ID NO: 43).

Natural variants of the human STAT3 gene product are known. Therefore, the ASOs of the present disclosure can be designed to reduce or inhibit expression of the natural variants of the STAT3 protein.

SEQ ID NO: 41 is identical to a STAT3 pre-mRNA sequence except that nucleotide “t” in SEQ ID NO: 41 is shown as “u” in pre-mRNA. In certain aspects, the “target nucleic acid” comprises an intron of a STAT3 protein-encoding nucleic acids or naturally occurring variants thereof, and RNA nucleic acids derived therefrom, e.g., pre-mRNA. In other aspects, the target nucleic acid comprises an exon region of a STAT3 protein-encoding nucleic acids or naturally occurring variants thereof, and RNA nucleic acids derived therefrom, e.g., pre-mRNA. In yet other aspects, the target nucleic acid comprises an exon-intron junction of a STAT3 protein-encoding nucleic acids or naturally occurring variants thereof, and RNA nucleic acids derived therefrom, e.g., pre-mRNA. The human STAT3 protein sequence encoded by the STAT3 pre-mRNA is shown as SEQ ID NO: 42. In other aspects, the target nucleic acid comprises an untranslated region of a STAT3 protein-encoding nucleic acids or naturally occurring variants thereof, e.g., 5′ UTR, 3′ UTR, or both.

In yet other aspects, the target nucleic acid comprises an exon-intron junction of a STAT3 protein-encoding nucleic acids or naturally occurring variants thereof, and RNA nucleic acids derived therefrom, e.g., pre-mRNA. The human STAT3 protein sequence encoded by the STAT3 pre-mRNA is shown as SEQ ID NO: 43. In other aspects, the target nucleic acid comprises an untranslated region of a STAT3 protein-encoding nucleic acids or naturally occurring variants thereof, e.g., 5′ UTR, 3′ UTR, or both.

In some aspects, an anti-STAT3 ASO of the disclosure hybridizes to a region within the introns of a STAT3 transcript, e.g., SEQ ID NO: 41 or SEQ ID NO: 43. In certain aspects, an anti-STAT3 ASO of the disclosure hybridizes to a region within the exons of a STAT3 transcript, e.g., SEQ ID NO: 41 or SEQ ID NO: 43. In other aspects, an anti-STAT3 ASO of the disclosure hybridizes to a region within the exon-intron junction of a STAT3 transcript, e.g., SEQ ID NO: 41 or SEQ ID NO: 43. In some aspects, an anti-STAT3 ASO of the disclosure hybridizes to a region within a STAT3 transcript (e.g., an intron, exon, or exon-intron junction), e.g., SEQ ID NO: 41 or SEQ ID NO: 43, wherein the anti-STAT3 ASO has a gapmer design.

In some aspects, the anti-STAT3 ASO targets an mRNA encoding a particular isoform of STAT3 protein (e.g., Isoform 1). In some aspects, the ASO targets all isoforms of STAT3 protein. In other aspects, the ASO targets two isoforms (e.g., Isoform 1 (UniProt ID: P40763-1) and Isoform 2 (UniProt ID: P40763-2), Isoform 2 and Isoform 3 (UniProt ID: P40763-3) of STAT3 protein.

In some aspects, an anti-STAT3 ASO of the disclosure hybridizes to a region within the introns of a STAT3 transcript, e.g., SEQ ID NO: 41 or SEQ ID NO: 43. In certain aspects, an anti-STAT3 ASO of the disclosure hybridizes to a region within the exons of a STAT3 transcript, e.g., SEQ ID NO: 41 or SEQ ID NO: 43. In other aspects, an anti-STAT3 ASO of the disclosure hybridizes to a region within the exon-intron junction of a STAT3 transcript, e.g., SEQ ID NO: 41 or SEQ ID NO: 43. In some aspects, an anti-STAT3 ASO of the disclosure hybridizes to a region within a STAT3 transcript (e.g., an intron, exon, or exon-intron junction), e.g., SEQ ID NO: 41 or SEQ ID NO: 43, wherein the ASO has a gapmer design.

In some aspects, the anti-STAT3 ASO of the present disclosure hybridizes to multiple target regions within the STAT3 transcript (e.g., genomic sequence, SEQ ID NO: 41). In some aspects, the ASO hybridizes to two different target regions within the STAT3 transcript. In some aspects, the anti-STAT3 ASO hybridizes to three different target regions within the STAT3 transcript. In some aspects, the anti-STAT3 ASOs that hybridizes to multiple regions within the STAT3 transcript (e.g., genomic sequence, SEQ ID NO: 41) are more potent (e.g., having lower EC50) at reducing STAT3 expression compared to anti-STAT3 ASOs that hybridizes to a single region within the STAT3 transcript (e.g., genomic sequence, SEQ ID NO: 41).

The anti-STAT3 ASOs of the disclosure comprise a contiguous nucleotide sequence which corresponds to the complement of a region of STAT3 transcript, e.g., a nucleotide sequence corresponding to SEQ ID NO: 41.

In some aspects, binding of an anti-STAT3 ASO targeting a STAT3 transcript disclosed herein to an mRNA transcript encoding STAT3 can reduce expression levels and/or activity levels of STAT3.

In some aspects, any anti-STAT3 ASO described herein can be part of an EV (e.g., exosome) of the present disclosure, i.e., an EV (e.g., exosome) a comprising construct comprising a cleavable linker disclosed herein, e.g., a construct of Formula I or II in which the biologically active moiety BAM is an anti-STAT3 ASO described herein or a combination thereof.

### ASO Targeting NRAS

In some aspects, the biologically active molecule BAM is an anti-NRas ASO. NRas is an oncogene encoding a membrane protein that shuttles between the Golgi apparatus and the plasma membrane. NRas-encoding genomic DNA can be found at Chromosomal position 1p13.2 (i.e., nucleotides 5001 to 17438 of GenBank Accession No. NG_007572). Specifically, a combination of time-lapse microscopy and photobleaching techniques have revealed that in the absence of palmitoylation, GFP-tagged N-Ras undergoes rapid exchange between the cytosol and ER/Golgi membranes, and that wild-type GFP—N-Ras is recycled to the Golgi complex by a nonvesicular mechanism. N-ras mutations have been described in melanoma, thyroid carcinoma, teratocarcinoma, fibrosarcoma, neuroblastoma, rhabdomyosarcoma, Burkitt lymphoma, acute promyelocytic leukemia, T cell leukemia, and chronic myelogenous leukemia. Oncogenic N-Ras can induce acute myeloid leukemia (AML) or chronic myelomonocytic leukemia (CMML)-like disease in mice.

Neuroblastoma RAS viral oncogene (NRas) is known in the art by various names. Such names include: GTPase NRas, N-ras protein part 4, neuroblastoma RAS viral (v-ras) oncogene homolog neuroblastoma RAS viral oncogene homolog, transforming protein N-Ras, and v-ras neuroblastoma RAS viral oncogene homolog.

The NRAS gene provides instructions for making a protein called N-Ras that is involved primarily in regulating cell division. The mRNA sequence encoding human NRAS can be found at NCBI Reference sequence NM_002524.5 and is represented by the coding sequence (SEQ ID NO: 53).

Natural variants of the human NRas gene product are known. For example, natural variants of human NRas protein can contain one or more amino acid substitutions selected from: G12D, G13D, T50I, G60E, and any combinations thereof. Additional variants of human NRas protein resulting from alternative splicing are also known in the art, such as: G13R, Q61K, Q61R, and P34L. Therefore, the anti-NRas ASOs of the present disclosure can be designed to reduce or inhibit expression of the natural variants of the STAT3 protein.

SEQ ID NO: 51 is identical to a NRas pre-mRNA sequence except that nucleotide “t” in SEQ ID NO: 51 is shown as “u” in pre-mRNA. In certain aspects, the “target nucleic acid” comprises an intron of a NRas protein-encoding nucleic acids or naturally occurring variants thereof, and RNA nucleic acids derived therefrom, e.g., pre-mRNA. In other aspects, the target nucleic acid comprises an exon region of a NRas protein-encoding nucleic acids or naturally occurring variants thereof, and RNA nucleic acids derived therefrom, e.g., pre-mRNA. In yet other aspects, the target nucleic acid comprises an exon-intron junction of a NRas protein-encoding nucleic acids or naturally occurring variants thereof, and RNA nucleic acids derived therefrom, e.g., pre-mRNA. The human NRas protein sequence encoded by the NRas pre-mRNA is shown as SEQ ID NO: 52. In other aspects, the target nucleic acid comprises an untranslated region of a NRas protein-encoding nucleic acids or naturally occurring variants thereof, e.g., 5′ UTR, 3′ UTR, or both.

In certain aspects, the anti-NRas ASOs of the disclosure also are capable of down-regulating (e.g., reducing or removing) expression of the NRas mRNA or protein. In this regard, the anti-NRas ASO of the disclosure can affect indirect inhibition of NRas protein through the reduction in NRas mRNA levels, typically in a mammalian cell, such as a human cell, such as a tumor cell. In particular, the present disclosure is directed to anti-NRas ASOs that target one or more regions of the NRas pre-mRNA (e.g., intron regions, exon regions, and/or exon-intron junction regions). Unless indicated otherwise, the term “NRas,” as used herein, can refer to NRas from one or more species (e.g., humans, non-human primates, dogs, cats, guinea pigs, rabbits, rats, mice, horses, cattle, and bears).

In some aspects, an anti-NRas ASO of the disclosure hybridizes to a region within the introns of a NRAS transcript, e.g., SEQ ID NO: 51 or SEQ ID NO: 53. In certain aspects, an ASO of the disclosure hybridizes to a region within the exons of a NRAS transcript, e.g., SEQ ID NO: 51 or SEQ ID NO: 53. In other aspects, an ASO of the disclosure hybridizes to a region within the exon-intron junction of a NRAS transcript, e.g., SEQ ID NO: 51 or SEQ ID NO: 53. In some aspects, an anti-NRas ASO of the disclosure hybridizes to a region within a NRAS transcript (e.g., an intron, exon, or exon-intron junction), e.g., SEQ ID NO: 51 or SEQ ID NO: 53, wherein the ASO has a gapmer design.

In some aspects, the anti-NRas ASO of the present disclosure hybridizes to multiple target regions within the NRas transcript (e.g., genomic sequence, SEQ ID NO: 51). In some aspects, the anti-NRas ASO hybridizes to two different target regions within the NRas transcript. In some aspects, the anti-NRas ASO hybridizes to three different target regions within the NRas transcript. In some aspects, the anti-NRas ASOs that hybridizes to multiple regions within the NRas transcript (e.g., genomic sequence, SEQ ID NO: 51) are more potent (e.g., having lower EC50) at reducing NRas expression compared to anti-NRas ASOs that hybridizes to a single region within the NRas transcript (e.g., genomic sequence, SEQ ID NO: 51).

In some aspects, the ASO targets an mRNA encoding a particular isoform of NRAS protein (e.g., Isoform 1, NCBI ID: NP_001229821.1). In some aspects, the ASO targets all isoforms of NRas protein. In other aspects, the ASO targets two isoforms (e.g., Isoform 1 and Isoform 2 (NCBI ID: NP_009089.4), Isoform 2 and Isoform 3 (NCBI ID: NP_001123995), and Isoform 3 and Isoform 4 (NCBI ID: NP_001229820.1)) of NRas protein.

The anti-NRas ASOs of the disclosure comprise a contiguous nucleotide sequence which corresponds to the complement of a region of NRas transcript, e.g., a nucleotide sequence corresponding to SEQ ID NO: 51.

In some aspects, binding of an anti-NRas ASO targeting a NRas transcript disclosed herein to an mRNA transcript encoding NRas can reduce expression levels and/or activity levels of NRas.

In some aspects, any anti-NRas ASO described herein can be part of an EV (e.g., exosome) of the present disclosure, i.e., an EV (e.g., exosome) a comprising construct comprising a cleavable linker disclosed herein, e.g., a construct of Formula I or II in which the biologically active moiety BAM is an anti-NRas ASO described herein or a combination thereof.

### ASO Targeting KRAS

In some aspects, the biologically active molecule BAM is an anti-KRAS ASO. The sequence for the human KRAS gene can be found at chromosomal location 12p12.1 and under publicly available GenBank Accession Number NC_000012 (25,204,789-25,250,936). The genomic sequence for human wild-type KRAS transcript corresponds to the reverse complement of residues 25,204,789-25,250,936 of NC_000012 (SEQ ID NO: 35). The KRAS G12D) genomic sequence provided in SEQ ID NO: 31 differs from SEQ ID NO: 35 in that it has a guanine to adenine substitution at nucleotide position 5,587. An exemplary KRAS G12D) mRNA sequence is provided in SEQ ID NO: 33, except that the nucleotide “t” in SEQ ID NO: 33 is shown as “u” in the mRNA. The KRAS G12D) mRNA provided in SEQ ID NO: 33 differs from the wild-type mRNA sequence (e.g., GenBank Accession No. NM_004985.5; SEQ ID NO: 37) in that it has a guanine to adenine substitution at nucleotide position 225. The sequence for human KRAS protein can be found under publicly available Accession Numbers: P01116 (canonical sequence), A8K8Z5, BOLPF9, P01118, and Q96D10, each of which is incorporated by reference herein in its entirety.

There are two isoforms of the human KRAS protein (P01116), resulting from alternative splicing. Isoform 2A (Accession Number: P01116-1; SEQ ID NO: 38) is the canonical sequence. It is also known as K-Ras4A. Isoform 2B (Accession Number: P01116-2; also known as K-Ras4B; SEQ ID NO: 36) differs from the canonical sequence as follows: (i) 151-153: RVE->GVD; and (ii) 165-189: QYRLKKISKEEKTPGCVKIKKCIIM (SEQ ID NO:599)->KHKEKMSKDGKKKKKKSKTKCVIM (SEQ ID NO:600). In some aspects, anti-KRAS ASOs disclosed herein can reduce or inhibit expression of KRAS protein Isoform 2A, Isoform 2B, or both.

Natural variants of the human KRAS gene product are known. For example, natural variants of human KRAS protein can contain one or more amino acid substitutions selected from: K5E, K5N, G10GG, G10V, G12A, G12C, G12F, G12I, G12L, G12R, G12S, G12V, G13C, G13D, G13E, G13R, G13V, V14I, L19F, T20M, Q22E, Q22H, Q22K, Q22R, Q25H, N26Y, F28L, E31K, D33E, P34L, P34Q, P34R, 136M, R41K, D57N, T58I, A59T, G60D, G60R, G60S, G60V, Q61A, Q61H, Q61K, Q61L, Q61P, Q61R, E63K, S65N, R68S, Y71H, T74A, L79I, R97I, Q99E, M11IL, K117N, K117R, D119G, S122F, T144P, A146P, A146T, A146V, K147E, K147T, R¹⁴⁹K, L₁₅₉S, I163S, R¹⁶⁴Q, 1183N, 184M, or combinations thereof. Natural variants that are specific to KRAS protein Isoform 2B contain one or more amino acid substitutions selected from: V152G, D₁₅₃V, F156I, F156L, or combinations thereof. The anti-KRAS ASOs of the present disclosure can be designed to reduce or inhibit expression of one or more of the variants of the KRAS protein (e.g., any variants known in the art). In some aspects, a KRAS mutant has an amino acid substitution of G12D. In some aspects, the anti-KRAS ASOs of the present disclosure target one or more KRAS mutants. In other aspects, a KRAS mutant that the anti-KRAS ASOs target is KRAS G12D (SEQ ID NO: 32). Exemplary sequences for KRAS G12D) mRNA and KRAS G12D protein are provided in SEQ ID NO: 33 and SEQ ID NO: 32.

In some aspects, a target nucleic acid sequence of an anti-KRAS ASO disclosed herein comprises one or more regions of a KRAS pre-mRNA. For example, SEQ ID NO: 31 (described above) is identical to a KRAS pre-mRNA sequence except that nucleotide “t” in SEQ ID NO: 31 is shown as “u” in the pre-mRNA. As used herein, the term “target nucleic acid sequence” refers to a nucleic acid sequence that is complementary to an anti-KRAS ASO disclosed herein. In certain aspects, the target nucleic acid sequence comprises an exon region of a KRAS protein-encoding nucleic acids or naturally occurring variants thereof, and RNA nucleic acids derived therefrom, e.g., pre-mRNA. In some aspects, the target nucleic acid sequence comprises an intron of a KRAS protein-encoding nucleic acids or naturally occurring variants thereof, and RNA nucleic acids derived therefrom, e.g., pre-mRNA. In further aspects, the target nucleic acid sequence comprises an exon-intron junction of a KRAS protein-encoding nucleic acids or naturally occurring variants thereof, and RNA nucleic acids derived therefrom, e.g., pre-mRNA. In some aspects, for example, when used in research or diagnostics, the target nucleic acid can be a cDNA or a synthetic oligonucleotide derived from DNA or RNA nucleic acid targets described herein. In some aspects, the target nucleic acid comprises an untranslated region of a KRAS protein-encoding nucleic acids or naturally occurring variants thereof, e.g., 5′ UTR, 3′ UTR, or both.

Accordingly, in some aspects, an anti-KRAS ASO disclosed herein hybridizes to an exon region of a KRAS transcript, e.g., SEQ ID NO: 31 or SEQ ID NO: 33. In some aspects, an anti-KRAS ASO of the present disclosure hybridizes to an intron region of a KRAS transcript, e.g., SEQ ID NO: 31. In some aspects, an anti-KRAS ASO hybridizes to an exon-intron junction of a KRAS transcript, e.g., SEQ ID NO: 31. In some aspects, an anti-KRAS ASO of the present disclosure hybridizes to a region within a KRAS transcript (e.g., an intron, exon, or exon-intron junction), e.g., SEQ ID NO: 31.

In some aspects, a target nucleic sequence of the ASOs disclosed herein is a KRAS mRNA, e.g., SEQ ID NO: 33. Accordingly, in certain aspects, an anti-KRAS ASO disclosed herein can hybridize to one or more regions of a KRAS mRNA. In some aspects, anti-KRAS ASOs of the present disclosure target mRNA encoding a particular isoform of KRAS protein. In certain aspects, anti-KRAS ASOs disclosed herein can target all isoforms of KRAS protein, including any variants thereof (e.g., those described herein). In some aspects, a KRAS protein that can be targeted by anti-KRAS ASOs of the present disclosure comprises a G12D amino acid substitution.

In some aspects, binding of an anti-KRAS ASO targeting a KRAS transcript disclosed herein to an mRNA transcript encoding KRAS can reduce expression levels and/or activity levels of KRAS.

In some aspects, any anti-KRAS ASO described herein can be part of an EV (e.g., exosome) of the present disclosure, i.e., an EV (e.g., exosome) a comprising construct comprising a cleavable linker disclosed herein, e.g., a construct of Formula I or II in which the biologically active moiety BAM is an anti-KRAS ASO described herein or a combination thereof.

### ASO Targeting Pmp22

In some aspects, the biologically active molecule BAM is an anti-Pmp22 ASO. Peripheral myelin protein 22 (PMP22) is also known as growth arrest-specific protein 3 (GAS-3), is encoded by the PMP22 gene. PMP22 is a 22 kDa transmembrane glycoprotein made up of 160 amino acids, and is mainly expressed in the Schwann cells of the peripheral nervous system. Schwann cells show high expression of PMP22, where it can constitute 2-5% of total protein content in compact myelin. Compact myelin is the bulk of the peripheral neuron's myelin sheath, a protective fatty layer that provides electrical insulation for the neuronal axon. The level of PMP22 expression is relatively low in the central nervous system of adults. PMP22 plays an essential role in the formation and maintenance of compact myelin. When Schwann cells come into contact with a neuronal axon, expression of PMP22 is significantly up-regulated, whereas PMP22 is down-regulated during axonal degeneration or transection. PMP22 has shown association with zonula-occludens 1 and occludin, proteins that are involved in adhesion with other cells and the extracellular matrix, and also support functioning of myelin. Along with cell adhesion function, PMP22 is also up-regulated during Schwann cell proliferation, suggesting a role in cell-cycle regulation. PMP22 is detectable in non-neural tissues, where its expression has been shown to serve as growth-arrest-specific (gas-3) function.

Improper gene dosage of the PMP22 gene can cause aberrant protein synthesis and function of myelin sheath. Since the components of myelin are stoichiometrically set, any irregular expression of a component can cause destabilization of myelin and neuropathic disorders. Alterations of PMP22 gene expression are associated with a variety of neuropathies, such as Charcot-Marie-Tooth type 1A (CMTIA), Dejerine-Sottas disease, and Hereditary Neuropathy with Liability to Pressure Palsy (HNPP). Too much PMP22 (e.g., caused by gene duplication) results in CMTIA. Gene duplication of PMP22 is the most common genetic cause of CMT where the overproduction of PMP22 results in defects in multiple signaling pathways and dysfunction of transcriptional factors like KNOX20, SOX10 and EGR2.

The sequence for the human PMP22 gene can be found under publicly available as NCBI RefSeq Acc. No. NM_000304. Alternative RefSeq mRNA transcripts have accession numbers NM_001281455, NM-001281456, NM-153321, and NM_153322, respectively. The human PMP22 gene is found at chromosome location 17p12 at 15,229,777-15,265,326.

The sequence for the human PMP22 pre-mRNA transcript (SEQ ID NO: 264) corresponds to the reverse complement of residues 15,229,777-15,265,326, of chromosome location 17p12. The PMP22 mRNA sequence (GenBank Accession No. NM_000304.4) is provided in SEQ ID NO: 58. The sequence for human PMP22 protein can be found under publicly available Uniprot Accession Number Q01453 (canonical sequence, SEQ ID NO: 60). Potential PMP22 isoforms have Uniprot Accession Numbers A8MU75, J3KQWO, A0A2R⁸Y5L5, J3KT36, and J3QS08, respectively. The publicly available contents of the database entries corresponding to accession numbers disclosed herein are incorporated by reference in their entireties.

The anti-PMP22 ASOs of the present disclosure can be designed to reduce or inhibit expression of the natural variants of the PMP22 protein.

An example of a target nucleic acid sequence of the anti-PMP22 ASOs is PMP22 pre-mRNA. SEQ ID NO: 58 represents a human PMP22 genomic sequence (i.e., reverse complement of nucleotides 15,229,777-15,265,326, complement, of chromosome 17p12). SEQ ID NO: 58 is identical to a PMP22 pre-mRNA sequence except that nucleotide “t” in SEQ ID NO: 58 is shown as “u” in pre-mRNA.

In some aspects, the anti-PMP22 ASO comprises a contiguous nucleotide sequence of 10 to 30 nucleotides in length that is complementary to a nucleic acid sequence within nucleotides 1 to 1828 of a PMP22 transcript corresponding to a nucleotide sequence as set forth in SEQ ID NO: 264 (PMP22 full mRNA transcript) or nucleotides 208 to 690 of a PMP22 transcript corresponding to a nucleotide sequence as set forth in SEQ ID NO: 59 (PMP22 coding sequence).

In some aspects, the contiguous nucleotide sequence is at least 80%, at least 85%, at least 90%, at least 95%, or about 100% complementary to the nucleic acid sequence within the PMP22 transcript. In some aspects, the anti-PMP22 ASO is capable of reducing PMP22 protein expression in a human cell (e.g., a Schwan cell), wherein the human cell expresses the PMP22 protein.

In some aspects, the PMP22 protein expression is reduced by at least 30%, at least 35%, at least 40%, at least 45%, at least 50%, at least 55%, at least 60%, at least 65%, at least 70%, at least 75%, at least 80%, at least 85%, at least 90%, at least 95%, or about 100% compared to PMP22 protein expression in a human cell that is not exposed to the anti-PMP22 ASO.

In some aspects, the anti-PMP22 ASO is capable of reducing a level of PMP22 mRNA in a human cell (e.g., an immune cell), wherein the human cell expresses the PMP22 mRNA. In some aspects, the level of PMP22 mRNA is reduced by at least 30%, at least 35%, at least 40%, at least 45%, at least 50%, at least 55%, at least 60%, at least 65%, at least 70%, at least 75%, at least 80%, at least 85%, at least 90%, at least 95%, or about 100% compared to the level of the PMP22 mRNA in a human cell that is not exposed to the anti-PMP22 ASO.

In certain aspects, the target nucleic acid comprises an intron of a PMP22 protein-encoding nucleic acids or naturally occurring variants thereof, and RNA nucleic acids derived therefrom, e.g., pre-mRNA. In other aspects, the target nucleic acid comprises an exon region of a PMP22 protein-encoding nucleic acids or naturally occurring variants thereof, and RNA nucleic acids derived therefrom, e.g., pre-mRNA. In yet other aspects, the target nucleic acid comprises an exon-intron junction of a PMP22 protein-encoding nucleic acids or naturally occurring variants thereof, and RNA nucleic acids derived therefrom, e.g., pre-mRNA. In some aspects, for example when used in research or diagnostics the target nucleic acid can be a cDNA or a synthetic oligonucleotide derived from the above DNA or RNA nucleic acid targets. The human PMP22 coding sequence (CDS) is shows as SEQ ID NO: 59, and protein sequence encoded by the coding sequence in the PMP22 pre-mRNA is shown as SEQ ID NO: 60. In other aspects, the target nucleic acid comprises an untranslated region of a PMP22 protein-encoding nucleic acids or naturally occurring variants thereof, e.g., 5′ UTR, 3′ UTR, or both.

In some aspects, an anti-PMP22 ASO of the disclosure hybridizes to a region within the introns of a PMP 22 transcript, e.g., SEQ ID NO: 58. In certain aspects, an ASO of the disclosure hybridizes to a region within the exons of a PMP22 transcript, e.g., SEQ ID NO: 58. In other aspects, an anti-PMP22 ASO of the disclosure hybridizes to a region within the exon-intron junction of a PMP22 transcript, e.g., SEQ ID NO: 58.

In some aspects, any anti-PMP22 ASO described herein can be part of an EV (e.g., exosome) of the present disclosure, i.e., an EV (e.g., exosome) a comprising construct comprising a cleavable linker disclosed herein, e.g., a construct of Formula I or II in which the biologically active moiety BAM is an anti-PMP22 ASO described herein or a combination thereof.

In some aspects, the construct of Formula I or II comprises ASO targeting STAT6 as the BAM conjugated at either the 5′ or 3′ end and L₁, L₂, and/or L₃ comprises a phosphodiester bond, a disulfido bond, and a cell penetrating peptide. In some aspects, the construct of Formula I or II comprises ASO as the BAM conjugated at either the 5′ or 3′ end and two of L₁, L₂, and L₃ comprise a disulfido bond and the third cleavable linker comprises a cell penetrating peptide. In some aspects, the construct of Formula I or II comprises ASO as the BAM conjugated at either the 5′ or 3′ end and L₁, L₂, and/or L₃ comprises a disulfido bond, a peptidyl, such as Val-Cit peptidyl, and a cell penetrating peptide. In any of the foregoing examples, the anchoring moiety AM is formed from a sterol, such as cholesterol, including cholesterol-TEG or thiocholesterol. In any of the foregoing examples, the AM is formed from dipalmitoylphosphatidic acid. In any of the foregoing examples, at leas tone of SP₁, SP₂, SP₃, and SP₄ comprises C₁₋₆ alkylenyl (e.g., C₂ alkylenyl, C₆ alkylenyl, or C₅ alkylenyl), polyoxyalkenyl (e.g., a polyoxyalkylenyl that comprises 2 to 15 —OCH₂CH₂-repeat units), a carbamoyl, an amino, an amido, a thiosuccinimido, a 1,2,3-triazolydibenzoylcyclooctenyl, a 1,2,3-triazolylbicyclononenyl, or a combination thereof, wherein the 1,2,3-triazolydibenzoylcyclooctenyl has the structure

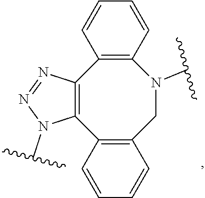

and
the 1,2,3-triazolylbicyclononenyl the structure:

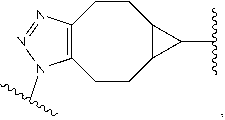

wherein denotes connectivity to AM, BAM, L₁, L₂, L₃, or the remainder of the spacer.

In some aspects, Formula I or II is a construct selected from

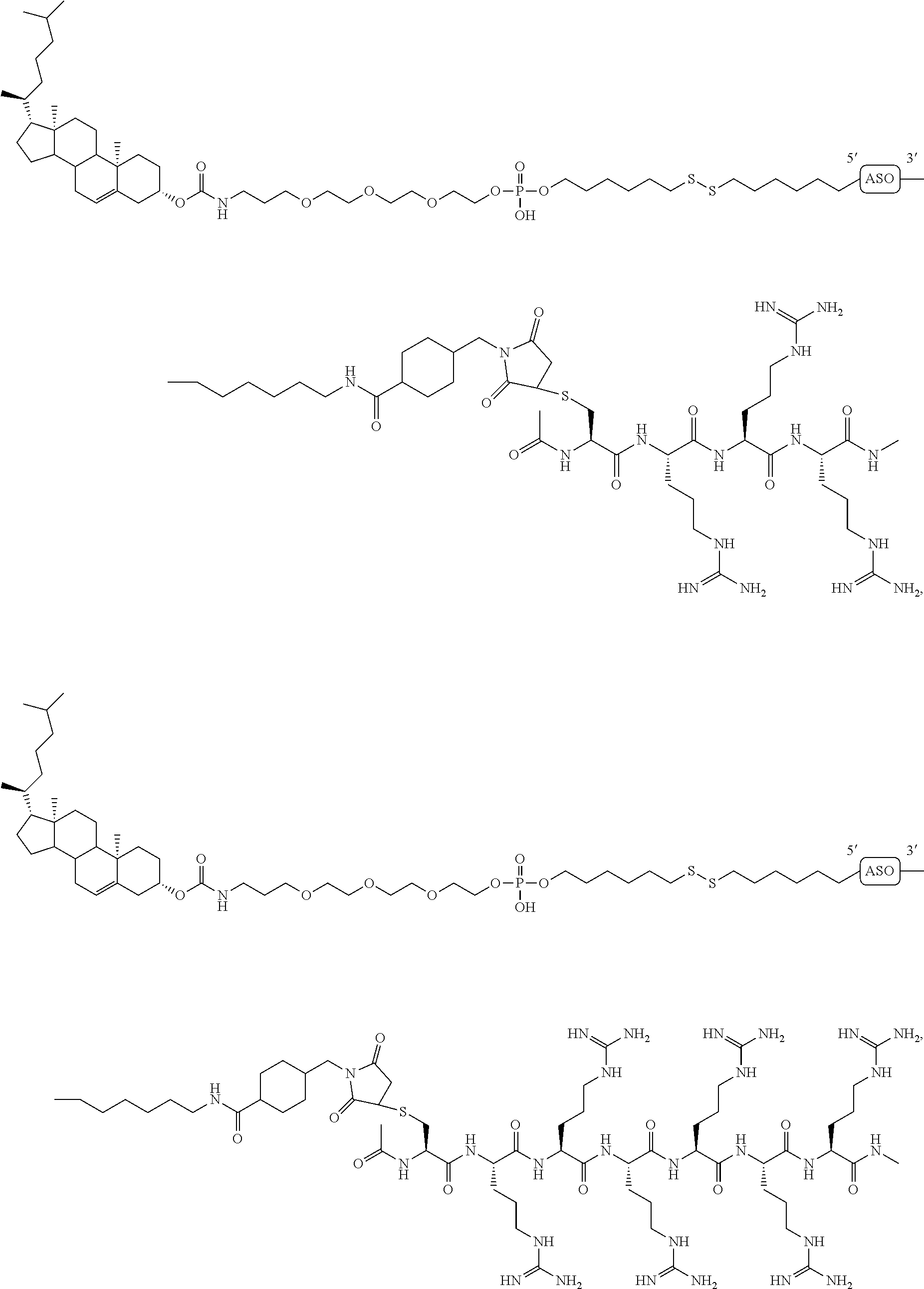

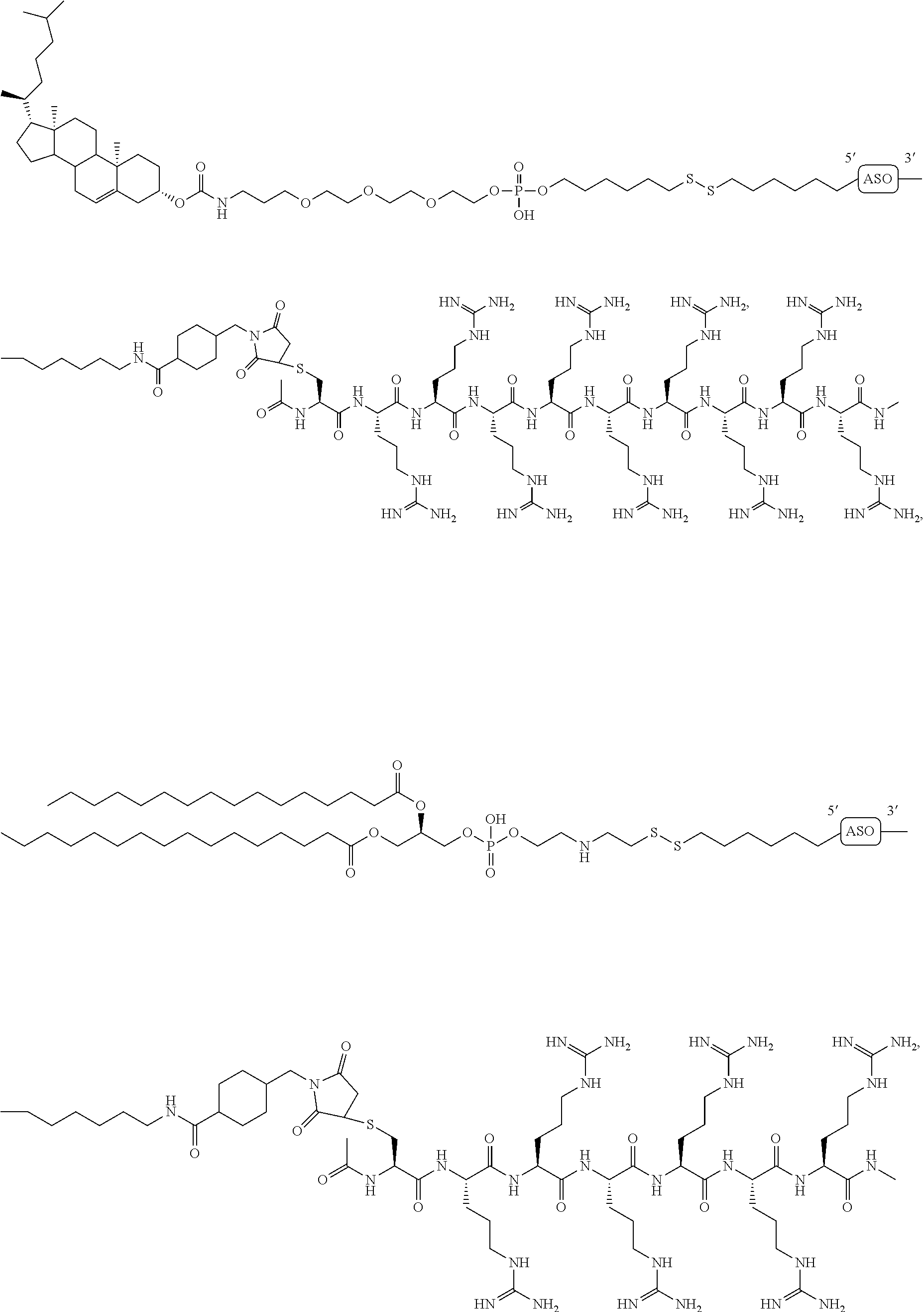

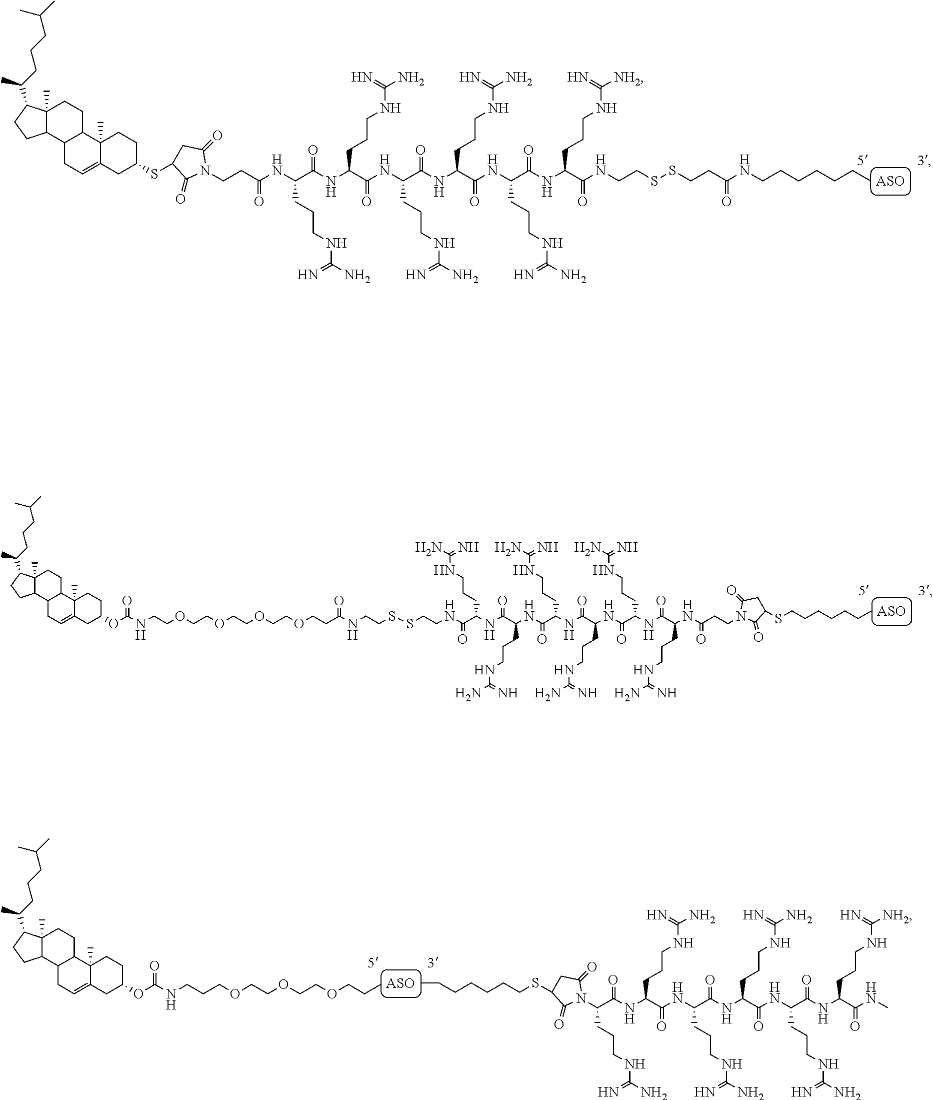

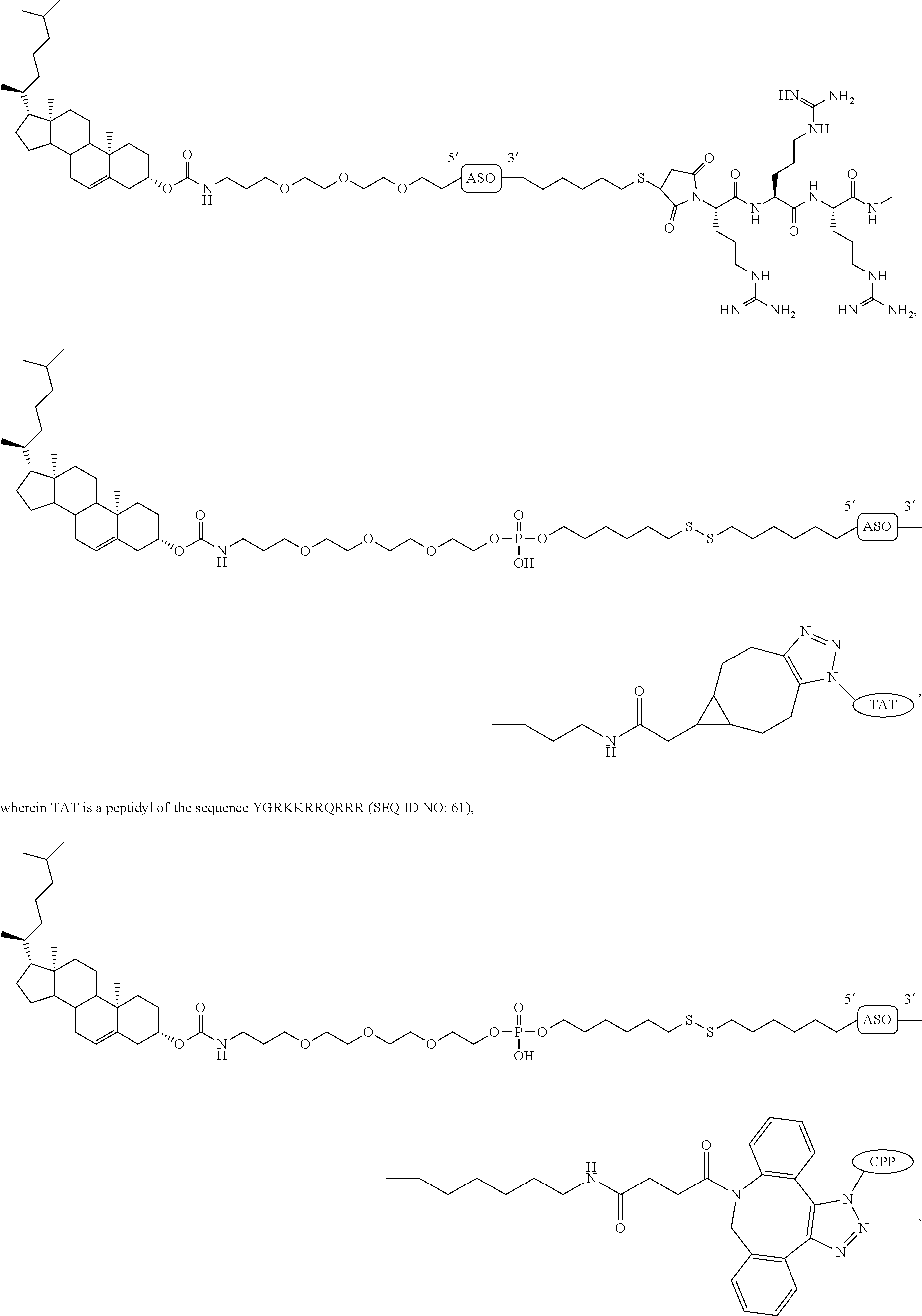

wherein the cell-penetrating peptide (CPP) is Antp (a peptidyl of the sequence RQIKIWFQNRRMKWKK (SEQ ID NO: 62)), R6 (a peptidyl of the sequence RRRRRR (SEQ ID NO: 87)), or cTAT (a peptide of sequence KRRRGRKKRRE (wherein K and E are connected to form a cyclic peptide)(SEQ ID NO: 88)), and

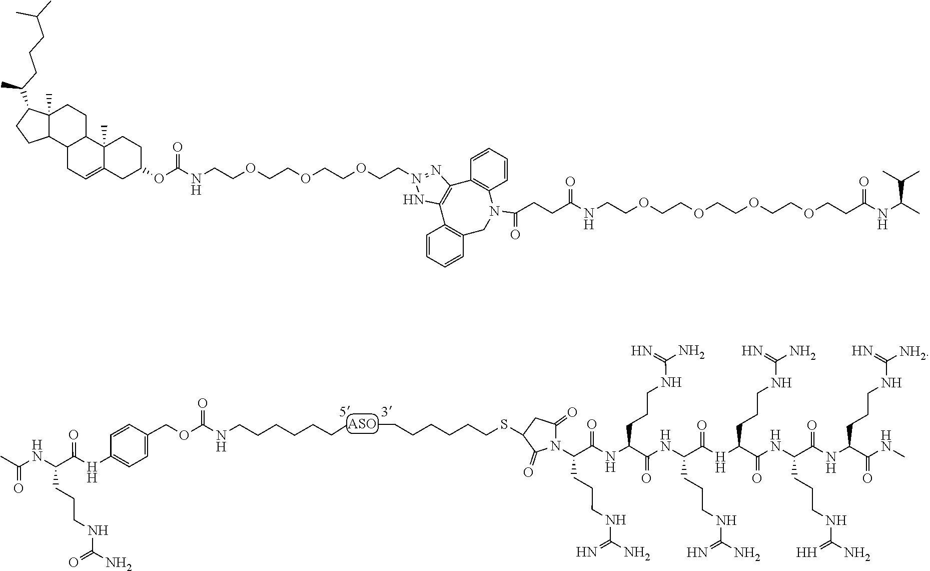

In some aspects, the EV is an exosome, e.g., a native exosome or a recombinant exosome. In some aspects wherein the exosome is a native exosome, the load density of ASO attached to the exosome is increased by at least 1.5-fold with respect to a control (see above). In some aspects wherein the exosome is an exosome overexpressing PTGFRN, the load density of ASO attached to the exosome is increased by at least 2-fold with respect to a control, e.g., a construct without a cleavable linker such as AM-BAM or AM-SP₁-BAM.

In some aspects wherein the exosome is a native exosome and the anchoring moiety AM (e.g., cholesterol, tocopherol, or palmitate), the average number of ASO molecules per native exosome is between about 500 and about 10,000. In some aspects, the average number of ASO molecules per native exosome is between about 1,000 and about 7,000. In some aspects, the average number of ASO molecules per native exosome is between about 700 and about 9,500, between about 800 and about 9,000, between about 850 and about 8,500, between about 900 and about 8,000, between about 950 and about 7,500, or between about 1,000 and about 7,000. In some aspects, the average number of ASO molecules per native exosome is at least 500, at least 600, at least 700, at least 800, at least 850, at least 900, at least 950, at least 1000, at least 1050, at least 1100, at least 1150, at least 1200, at least 1250, or at least 1300 and/or 10,000 or fewer, 9,000 or fewer, 8,000 or fewer, 7,500 or fewer, 7,000 or fewer, 6,500 or fewer, 6,000 or fewer, 5,500 or fewer, 5,000 or fewer, 4,500 or fewer, 4,000 or fewer, 3,500 or fewer, 3,000 or fewer, 2,500 or fewer, 2,000 or fewer, 1,500 or fewer, or 1,000 or fewer. In some aspects, the loading efficiency of the native exosome is between about 70% and about 95%. In some aspects, the loading efficiency of the native exosome is between about 70% and about 75%, between about 75% and about 80%, between about 80% and about 85%, between about 85% and about 90%, or between about 90% and about 95%. In some aspects, the loading efficiency of the native exosome is at least 70%, at least 75%, at least 80%, at least 85%, at least 90%, or at least 95%.

In some aspects wherein the exosome is a Scaffold-X exosome and the anchoring moiety AM is cholesterol, the average number of ASO molecules per exosome is 2442+/−339. In some aspects, the average number of ASO molecules per Scaffold-X exosome is between about 2000 and about 3000. In some aspects, the average number of ASO molecules per Scaffold-X exosome is between about 2000 and about 2100, between about 2100 and about 2200, between about 2200 and about 2300, between about 2300 and about 2400, between about 2400 and about 2500, between about 2500 and about 2600, between about 2600 and about 2700, between about 2700 and about 2800, between about 2800 and about 2900, or between about 2900 and about 3000. In some aspects, the average number of ASO molecules per Scaffold-X exosome is at least 2000, at least 2100, at least 2200, at least 2300, at least 2400, at least 2500, at least 2600, at least 2700, at least 2800, at least 2900, or at least 3000. In some aspects, the loading efficiency of the Scaffold-X exosome is 27% to 46%. In some aspects, the loading efficiency of the Scaffold-X exosome is between about 25% and about 50%. In some aspects, the loading efficiency of the Scaffold-X exosome is between about 25% and about 30%, between about 30% and about 35%, between about 35% and about 40%, between about 40% and about 45%, or between about 45% and about 50%. In some aspects, the loading efficiency of the Scaffold-X exosome is at least 25%, at least 30%, at least 35%, at least 40%, at least 45%, or at least 50%.

In some aspects, the average number of ASO molecules per Scaffold-X exosome is more than 5000, more than 6000, more than 7000, more than 8000, more than 9000, more than 10,000, more than 11,000, more than 12,000, more than 13,000, more than 14,000, more than 15,000, more than 16,000, more than 17,000, more than 18,000, more than 19,000, or more than 20,000. In some aspects, the average number of ASO molecules per Scaffold-X exosome is between about 5000 and about 6,000, about 6,000 and about 7,000, about 7,000 and about 8,000, about 8,000 and about 9,000, about 9,000 and about 10,000, about 10,000 and about 11,000, about 11,000 and about 12,000 about 12,000 and about 13,000, about 13,000 and about 14,000, about 14,000 and about 15,000, about 15,000 and about 16,000, about 16,000 and about 17,000, about 17,000 and about 18,000, about 18,000 and about 19,000, or about 19,0000 and about 20,000.

### Methods of Making

The present disclosure provides a method of attaching a biologically active molecule BAM to an EV (e.g., an exosome) comprising linking an anchoring moiety AM to the EV, wherein the anchoring moiety AM is attached to the biologically active moiety BAM according to Formula I or II:

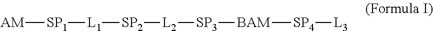

wherein L₁, L₂, and L₃ are the same or different and each is an optional cleavable linkage; and SP₁, SP₂, SP₃, and SP₄ are optional first, second, third, and fourth spacers, respectively, and wherein at least one of L₁, L₂, and L₃ is present and comprises a cell penetrating peptide. In some aspects, BAM is an antisense oligonucleotide (ASO), e.g., a gapmer.

The present disclosure also provides a method of increasing the load density of a biologically active molecule BAM attached to an EV, comprising screening a library of anchoring moieties AM attached to the biologically active moiety BAM according to Formula I or II:

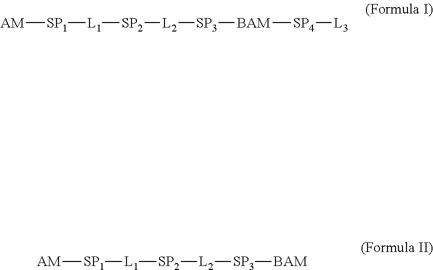

wherein L₁, L₂, and L₃ are the same or different and each is an optional cleavable linkage; and SP₁, SP₂, SP₃, and SP₄ are optional first, second, third, and fourth spacers, respectively, and wherein at least one of L₁, L₂, and L₃ is present and comprises a cell penetrating peptide. In some aspects, BAM is an antisense oligonucleotide (ASO), e.g., a gapmer. In some aspects, the load density of a biologically active molecule BAM attached to an EV, e.g., an exosome, is increased at least 1-fold, at least 1.5-fold, at least 2-fold, at least 2.5-fold, at least 3-fold, at least 3.5-fold, at last about 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold with respect to a control. In some aspects, the control is a corresponding construct lacking the cleavable linker (e.g., L₁, L₂, and/or L₃). For example, in some aspects, the control for a construct of Formula I or II, is a control construct with the structure AM-BAM or AM-SP₁-BAM.

EVs (e.g., exosomes) of the present disclosure can be produced by chemical synthesis, recombinant DNA technology, biochemical or enzymatic fragmentation of larger molecules, combinations of the foregoing or by any other method. In one aspect, the present disclosure provides a method of attaching a biologically active molecule to an EV (e.g., exosome) via a cleavable linker disclosed herein, e.g., via solid phase synthesis or conjugation.

Exosome production: In some aspects, EVs disclosed herein (e.g., exosomes) can be produced from a cell grown in vitro or a body fluid of a subject. When exosomes are produced from in vitro cell culture, various producer cells, e.g., HEK293 cells, CHO cells, and MSCs, can be used. In some aspects, a producer cell is not a dendritic cell, macrophage, B cell, mast cell, neutrophil, Kupffer-Browicz cell, cell derived from any of these cells, or any combination thereof.

In certain aspects, the producer cell is HEK293 cells. Human embryonic kidney 293 cells, also often referred to as HEK 293, HEK-293, 293 cells, or less precisely as HEK cells, are a specific cell line originally derived from human embryonic kidney cells grown in tissue culture.

A comprehensive study of the genomes and transcriptomes of HEK 293 and five derivative cell lines compared the HEK 293 transcriptome with that of human kidney, adrenal, pituitary and central nervous tissue. The HEK 293 pattern most closely resembled that of adrenal cells, which have many neuronal properties. HEK 293 cells have a complex karyotype, exhibiting two or more copies of each chromosome and with a modal chromosome number of 64. They are described as hypotriploid, containing less than three times the number of chromosomes of a haploid human gamete. Chromosomal abnormalities include a total of three copies of the X chromosome and four copies of chromosome 17 and chromosome 22. Variants of HEK293 cells useful to produce EVs include, but are not limited to, HEK 293F, HEK 293 FT, and HEK 293T.

Solid-phase synthesis: Solid phase synthesis known in the art can additionally or alternatively be employed to generate the constructs disclosed in the present application. In some aspects, two or more components of a cleavable linker disclosed herein can be attached to each other (e.g., concatenated) using solid phase synthesis. For example, an ASO can be synthesized, and different spacers or combinations thereof can be added to the ASO via conventional synthetic steps. A spacer, such as C₃-phosphoramidite, TEG-phosphoramidite, or HEG-phosphoramidite cam be used. In some aspects, the spacer or combination of spacers can be further extended via synthesis to incorporate the membrane anchor moiety. In an example, phosphoramidites can be used to generate the optimized linkers of the present disclosure via solid phase synthesis such as octyl-tocopherol phosphoramidite, tocopherol phosphoramidite, palmitate-C₆ phosphoramidite, cholesterol-TEG phosphoramidite or or cholesterol-C₆ phosphoramidite. Suitable solid phase techniques, including automated synthesis techniques, are described, e.g., in F. Eckstein (ed.), Oligonucleotides and Analogues, a Practical Approach, Oxford University Press, New York (1991) and Toy, P.H.; Lam, Y (ed.), Solid-Phase Organic synthesis, concepts, Strategies, and Applications, John Wiley & Sons, Inc. New Jersey (2012).

Conjugation: In some aspects, two or more components of a linker disclosed herein can be attached to each other (e.g., concatenated) using conjugation. Besides amine-reactive compounds, those having chemical groups that form bonds with sulfhydryls (—SH) can be used as crosslinkers and modification reagents for protein and other bioconjugate techniques. Sulfhydryls, also called thiols, exist in proteins in the side-chain of cysteine (Cys, C) amino acids.

Sulfhydryl groups are useful targets for protein conjugation and labeling. First, sulfhydryls are present in most proteins but are not as numerous as primary amines; thus, crosslinking via sulfhydryl groups is more selective and precise. Second, sulfhydryl groups in proteins are often involved in disulfide bonds, so crosslinking at these sites typically does not significantly modify the underlying protein structure or block binding sites. Third, the number of available (i.e., free) sulfhydryl groups can be easily controlled or modified; they can be generated by reduction of native disulfide bonds, or they can be introduced into molecules through reaction with primary amines using sulfhydryl-addition reagents, such as 2-iminothiolane (Traut's Reagent), N-succinimidyl S-acetylthioacetate (SATA), N-succinimidyl S-acetylthiopropionate (SATP), or N-Succinimidyl S-acetyl (thiotetraethylene glycol)(SAT (PEG)). Finally, combining sulfhydryl-reactive groups with amine-reactive groups to make heterobifunctional crosslinkers provides greater flexibility and control over crosslinking procedures. For example, using 3-maleimido-propionic N-hydroxysuccinimide (NHS) ester, which contains a maleimide group and an NHS ester, the NHS ester can be used to label the primary amines (-NH₂) of proteins, amine-modified oligonucleotides, and other amine-containing molecules. The maleimide group will react with a thiol group to form a covalent bond, enabling the connection of biomolecule with a thiol.

The maleimide group reacts specifically with sulfhydryl groups when the pH of the reaction mixture is between 6.5 and 7.5; the result is formation of a stable thioether linkage that is not reversible (i.e., the bond cannot be cleaved with reducing agents). In more alkaline conditions (pH>8.5), the reaction favors primary amines and also increases the rate of hydrolysis of the maleimide group to a non-reactive maleamic acid. Maleimides do not react with tyrosines, histidines or methionines.

Thiol-containing compounds, such as dithiothreitol (DTT) and beta-mercaptoethanol (BME), must be excluded from reaction buffers used with maleimides because they will compete for coupling sites. For example, if DTT were used to reduce disulfides in a protein to make sulfhydryl groups available for conjugation, the DTT would have to be thoroughly removed using a desalting column before initiating the maleimide reaction. Interestingly, the disulfide-reducing agent tris(2-carboxyethyl) phosphine (TCEP) does not contain thiols and does not have to be removed before reactions involving maleimide reagents.

Excess maleimides can be quenched at the end of a reaction by adding free thiols. ethylenediaminetetraacetic acid (EDTA) can be included in the coupling buffer to chelate stray divalent metals that otherwise promote oxidation of sulfhydryls (non-reactive).

In one aspect, the linking comprises treating the EV (e.g., exosome) with a reducing agent. Suitable reducing agents include, for example, TCEP (tris(2-carboxyethyl) phosphine), DTT (dithiothreitol), BME (2-mercaptoethanol), a thiolating agent, and any combination thereof. The thiolating agent can comprise, e.g., Traut's reagent (2-iminothiolane).

After the treatment with the reducing agent, the linking reaction further comprises bringing the reduced EV (e.g., exosome) in contact with the maleimide moiety. In one aspect, the maleimide moiety is linked to a biologically active molecule prior to the linking to the EV (e.g., exosome). In some aspects, the maleimide moiety is further attached to a linker to connect the maleimide moiety to the biologically active molecule. Accordingly, in some aspects, one or more linkers or spacers are interposed between the maleimide moiety and the biologically active molecule.

Any of the anchoring moieties AM, spacer SP or spacer combinations, or biologically active molecules BAM disclosed herein can be conjugated to a reactive moiety, e.g., an amino reactive moiety (e.g., NHS-ester, p-nitrophenol, isothiocyanate, isocyanate, or aldehyde), a thiol reactive moiety (e.g., acrylate, maleimide, or pyridyl disulfide), a hydroxy reactive moiety (e.g., isothiocyanate or isocyanate), a carboxylic acid reactive moiety (e.g., epoxide), or an azide reactive moiety (e.g., alkyne). Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

Exemplary reactive groups that can be used to covalently bind two components disclosed herein (e.g., an anchoring moiety AM and a spacer SP, two spacers SP, a spacer SP and a biologally active molecule BAM, or an anchoring moiety AM and a biologically acitve moiety BAM) via chemical conjugation include, e.g., N-succinimidyl-3-(2-pyridyldithio) propionate, N-4-maleimide butyric acid, S-(2-pyridyldithio) cysteamine, iodoacetoxysuccinimide, N-(4-maleimidebutyryl oxy) succinimide, N-[5-(3′-maleimide propylamide)-1-carboxypentyl] iminodiacetic acid, N-(5-aminopentyl) iminodiacetic acid, and 1′-[(2-cyanoethyl)-(N,N-diisopropyl)]-phosphoramidite). Bifunctional linkers (linkers containing two reactive groups) are also usable.

In some aspects, an anchoring moiety AM, spacer SP, or biologically active molecule BAM can comprise a terminal oxyamino group (—ONH₂), a hydrazino group (—NHNH₂), a mercapto group (i.e., SH or thiol), or an olefin (e.g., —CH—CH₂). In some aspects, an anchoring moiety AM, spacer SP, or biologically active molecule BAM can comprise e.g., at a terminal position, an electrophilic moiety, such as an aldehyde, alkyl halide, mesylate, tosylate, nosylate, or brosylate, or an activated carboxylic acid ester (e.g., an NHS ester, a phosphoramidite, or a pentafluorophenyl ester).

The term “protecting group,” as used herein, refers to a labile chemical moiety which is known in the art to protect reactive groups, including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999).

Additionally, the various synthetic steps can be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Amine reactive moieties: In some aspects, the reactive moiety is an amine reactive moiety. As used herein the term “amine reactive moiety” refers to a chemical group that can react with a reactive group having an amino moiety, e.g., a primary amine. Exemplary amine reactive moieties are N-hydroxysuccinimide esters (NHS-ester), p-nitrophenol, isothiocyanate, isocyanate, and aldehyde. Alternative reactive moieties that react with primary amines are also well known in the art. In some aspects, an amine reactive moiety can be attached to a terminal position of an anchoring moiety AM, spacer SP, or biologically active molecule BAM of the present disclosure. In some aspects, the amine reactive moiety is a NHS-ester. Typically, a NHS-ester reactive moiety reacts with a primary amine of a reactive group to yield a stable amide bond and N-hydroxysuccinimide (NHS). In some aspects, the amine reactive moiety is a p-nitrophenol group. Typically, a p-nitrophenol reactive moiety is an activated carbamate that reacts with a primary amine of a reactive group to yield a stable carbamate moiety and p-nitrophenol. In some aspects, the amine reactive moiety is an isothiocyanate. Typically, an isothiocyanate reacts with a primary amine of a reactive group to yield a stable thiourea moiety. In some aspects, the amine reactive moiety is an isocyanate. Typically, an isocyanate reacts with a primary amine of a reactive group to yield a stable urea moiety. In some aspects, amine the reactive moiety is an aldehyde. Typically, aldehydes react with primary amines to form Schiff bases which can be further reduced to form a covalent bond through reductive amination.

Thiol reactive moieties: In some aspects, the reactive moiety is a thiol reactive moiety. As used herein the term “thiol reactive moiety” refers to a chemical group that can react with a reactive group having a thiol moiety (or mercapto group). Exemplary thiol reactive moieties are acrylates, maleimides, and pyridyl disulfides. Alternative reactive moieties that react with thiols are also well known in the art. In some aspects, a thiol reactive moiety can be attached to a terminal position of an anchoring moiety AM, spacer SP, or biologically active molecule BAM of the present disclosure. In some aspects, the thiol reactive moiety is an acrylate. Typically, acrylates react with thiols at the carbon B to the carbonyl of the acrylate to form a stable sulfide bond. In some aspects, the thiol reactive moiety is a maleimide. Typically, maleimides react with thiols at either the carbon B or the carbonyls to form a stable sulfide bond. In some aspects, the thiol reactive moiety is a pyridyl disulfide. Typically, pyridyl disulfides react with thiols at the sulfur atom β to the pyridyl to form a stable disulfide bond and pyridine-2-thione.

Hydroxy reactive moieties: In some aspects, the reactive moiety is a hydroxyl reactive moiety. As used herein the term “hydroxyl reactive moiety” refers to a chemical group that can react with a reactive group having a hydroxyl moiety. Exemplary hydroxyl reactive moieties are isothiocyanates and isocyanates. Alternative reactive moieties that react with hydroxyl moieties are also well known in the art. In some aspects, a hydroxyl reactive moiety can be attached to a terminal position of an anchoring moiety AM, spacer SP, or biologically active molecule BAM of the present disclosure. In some aspects, the hydroxyl reactive moiety is an isothiocyanate. Typically, an isothiocyanate reacts with a hydroxyl of a reactive group to yield a stable carbamothioate moiety. In some aspects, amine the reactive moiety is an isocyanate. Typically, an isocyanate reacts with a hydroxyl of a reactive group to yield a stable carbamate moiety.

Carboxylic acid reactive moieties: In some aspects, the reactive moiety is a carboxylic acid reactive moiety. As used herein the term “carboxylic acid reactive moiety” refers to a chemical group that can react with a reactive group having a carboxylic acid moiety. An exemplary carboxylic acid reactive moiety is an epoxide. Alternative reactive moieties that react with carboxylic acid moieties are also well known in the art. In some aspects, a carboxylic acid reactive moiety can be attached to a terminal position of an anchoring moiety AM, spacer SP, or biologically active molecule BAM of the present disclosure. In some aspects, the carboxylic acid reactive moiety is an epoxide. Typically, an epoxide reacts with the carboxylic acid of a reactive group at either of the carbon atoms of the epoxide to form a 2-hydroxyethyl acetate moiety.

Azide reactive moieties: In some aspects, the reactive moiety is an azide reactive moiety. As used herein the term “azide reactive moiety” refers to a chemical group that can react with a reactive group having an azide moiety. An exemplary azide reactive moiety is an alkyne. Alternative reactive moieties that react with azide moieties are also well known in the art. In some aspects, a carboxylic acid reactive moiety can be attached to a terminal position of an anchoring moiety AM, spacer SP, or biologically active molecule BAM of the present disclosure. In some aspects, the azide reactive moiety is an alkyne. Typically, an alkyne reacts with the azide of a reactive group through a 1,3-dipolar cycloaddition reaction, also referred to “click chemistry,” to form a 1,2,3-triazole moiety.

In some aspects, the exosomes disclosed herein can be prepared and/or stored under conditions that preserve the stability of the exosome and/or promote higher load density. In some aspects, an exosome comprising an ASO attached to its surface via a membrane anchoring construct of Formula I or II can be maintained in a low salt buffer (e.g., comprising about 150 mM NaCl) for about 2, 4, 6, or 8 days. In some aspects, an exosome comprising an ASO attached to its surface via a membrane anchoring construct of Formula I or II comprising cholesterol as the anchoring moiety can be maintained in a low salt buffer, high salt buffer, or high salt buffer (e.g., comprising about 150 mM NaCl) further comprising sucrose for about 2, 4, 6, or 8 days, at either 4° C. or 25° C.

The present disclosure also provides a method to increase the loading density of an exosome comprising loading the exosome under high salt conditions (e.g., using a high salt buffer comprising about 150 mM NaCl). In some aspects, the exosome is loaded with an ASO attached (e.g., covalently attached) via a membrane anchoring construct of Formula I or II.

### Therapeutic Uses

The present disclosure provides methods of treating a disease or condition in a subject in need thereof comprising administering a composition comprising EVs (e.g., exosomes) of the present disclosure to the subject. The present disclosure also provides methods of preventing or ameliorating the symptoms of a disease or condition is a subject in need thereof comprising administering a composition comprising EVs (e.g., exosomes) of the present disclosure to the subject. The present disclosure also provides methods to diagnose a disease or condition in a subject in need thereof comprising administering a composition comprising EVs (e.g., exosomes) of the present disclosure to the subject.

The present disclosure also provides methods of preventing and/or treating a disease or disorder in a subject in need thereof, comprising administering an EV (e.g., exosome) of the present disclosure to the subject. In some aspects, a disease or disorder that can be treated with the present methods comprises a cancer, graft-versus-host disease (GvHD), an autoimmune disease, an infectious disease, a fibrotic disease, an inflammatory disease, a neurodegenerative disorder, a central nervous disease, a muscular dystrophy disease, or a metabolic disease. In some aspects, the treatment is prophylactic. In other aspects, the EVs (e.g., exosomes) of the present disclosure are used to induce an immune response. In other aspects, the EVs (e.g., exosomes) for the present disclosure are used to vaccinate a subject.

In some aspects, the disease or disorder is a cancer. When administered to a subject with a cancer, in certain aspects, EVs (e.g., exosomes) of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors.” In some aspects, the cancer being treated is characterized by low levels or undetectable levels of leukocyte infiltration into the tumor microenvironment, or so-called “cold tumors” or “non-inflammatory tumors.” In some aspects, an EV (e.g., exosome) is administered in an amount and for a time sufficient to convert a “cold tumor” into a “hot tumor,” i.e., the administering results in the infiltration of leukocytes (such as T-cells) into the tumor microenvironment. In certain aspects, the cancer comprises bladder cancer, cervical cancer, renal cell cancer, breast cancer, prostate cancer, testicular cancer, colorectal cancer, lung cancer, head and neck cancer, ovarian cancer, lymphoma, pancreatic cancer, liver cancer, glioblastoma, melanoma, myeloma, leukemia, or combinations thereof. In other aspects, the terms “distal tumor” or “distant tumor” refer to a tumor that has spread from the original (or primary) tumor to distant organs or distant tissues, e.g., lymph nodes. In some aspects, the EVs (e.g., exosomes) of the disclosure can treat a tumor after the metastatic spread.

In some aspects, the disease or disorder is a graft-versus-host disease (GvHD).

In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, bullous pemphigoid, celiac disease, Devic's disease (neuromyelitis optica), glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, the disease or disorder that can be treated with the present disclosure is an inflammatory disease. Non-limiting examples of inflammatory diseases include inflammation, fatty liver disease, endometriosis, type I diabetes, type II diabetes, inflammatory bowel disease, asthma, rheumatoid arthritis, psoriatic arthritis, Gouty arthritis, obesity, chronic peptic ulcer, ulcerative colitis, sinusitis, active hepatits, psoriasis, chronic obstructive pulmonary disease (COPD), allergies, bronchitis, and appendicitis.

In some aspects, the disease or disorder that can be treated with the present disclosure is a central nervous system (CNS) disease. Non-limiting examples of CNS diseases include Alzheimer's disease, Bell's palsy, cerebral palsy, epilepsy, motor neurone disease, multiple sclerosis, neurofibromatoriss, Parkinson's disease, sciatica, shingles, stroke, transiet ischemic attack, subdural hemorrhage, hematoma, meningitis, encephalitis, polio, epidural abcess, cervical spondylosis, capral tunnel syndrome, peripheral neuropathy, Guillan-Barre syndrome, headache, neuralgia, amyotrophic lateral sclerosis, and Huntington chorea.

In some aspects, the disease or disorder that can be treated with the present disclosure is a fibrotic disease. Non-limiting examples of fibrotic diseases include pulmonary fibrosis, liver fibrosis, heart fibrosis, mediastinal fibrosis, bone marrow fibrosis, skin fibrosis, scleroderma, retroperitoneal cavity fibrosis, and keloids.

In some aspects, the disease or disorder is an infectious disease. In certain aspects, the disease or disorder is an oncogenic virus. In some aspects, infectious diseases that can be treated with the present disclosure includes, but not limited to, human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, a corona virus (e.g., MERS-CoV and SARS COV), a filovirus (e.g., Marburg and Ebola), Streptococcus pyogenes, Streptococcus pneumoniae, Plasmodia species (e.g., vivax and falciparum), Chikunga virus, human Papilloma virus (HPV), hepatitis B, hepatitis C, human herpes virus 8, herpes simplex virus 2 (HSV2), Klebsiella spp., Pseudomonas aeruginosa, Enterococcus spp., Proteus spp., Enterobacter spp., Actinomycetota (Actinobacter) sp., coagulase-negative staphylococci (CoNS), Mycoplasma sp., or combinations thereof.

In some aspects, a disease or disorder that can be treated with the present methods comprises Pompe disease, Gaucher disease, a lysosomal storage disorder, mucovicidosis, cystic fibrosis, Duchenne muscular dystrophy, Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, ornithine transcarbamylase (OTC) deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, al antitrypsin deficiency, Rett Syndrome, Dravet Syndrome, Angelman Syndrome, DM1 disease, Fragile X disease, Huntington's Disease, Friedreich's ataxia, Charcot-Marie-Tooth (CMT) disease (also known as hereditary motor and sensory neuropathy (HMSN) or peroneal muscular atrophy), CMT1X (Charcot-Marie-Tooth type 1X) disease, catecholaminergic polymorphic ventricular tachycardia, spinocerebellar ataxia type 3 (SCA3) disease, limb-girdle muscular dystrophy, or a hypercholesterolemia. In some aspects, the treatment of such disease or disorder is prophylactic.

In some aspects, the disease or disorder is a neurodegenerative disease. In some aspects, the neurodegenerative disease is selected from Alzheimer's disease, Parkinson's disease, a prion disease, a motor neuron disease, Huntington's disease, spinocerebellar ataxia, spinal muscular atrophy, and any combination thereof.

In certain aspects, the disease or disorder comprises a muscular dystrophy. In some aspects, the muscular dystrophy is selected from Duchenne type muscular dystrophy (DMD), myotonic muscular dystrophy, facioscapulohumeral muscular dystrophy (FSHD), congenital muscular dystrophy, limb-girdle muscular dystrophy (including, but not limited to, LGMD2B, LGMD2D, LGNMD2L, LGMD2C, LGMD2E and LGMD2A), and any combination thereof.

In some aspects, the disease or disorder is selected from aromatic L-amino acid decarboxylase (AADC) deficiency (CNS), adenosine deaminase severe combined immunodeficiency (ADA-SCID), Alpha-1 antitrypsin deficiency, β-thalassemia (severe sickle cell), Cancer (head and neck squamous cell), Niemman-Pick Type C Disease, Cerebral ALD, Choroideremia, Congestive heart failure, Cystic Fibrosis, Duchenne muscular dystrophy (DMD), Fabry disease, Glaucoma, Glioma (cancer), Hemophilia A, Hemophilia B, HoFH (hypercholesterolemia), Huntington's Disease, Lipoprotein lipase deficiency, Leber hereditary optic neuropathy (LHON), Metachromatic leukodystrophy, Mucopolysaccharidosis type I (MPS I-Hurler syndrome), Mucopolysaccharidosis type II (MPS II-Hunter's syndrome), Mucopolysaccharidosis type III (MPS III-Sanfilippo Syndrome), Mucopolysaccharidosis type IIIA (MPS IIIA), Parkinson's disease, Pompe Disease, Recessive Dystrophic Epidermolysis Bullosa, RPE65 gene deficiency (vision loss), spinal muscular atrophy (SMA I), wet age-related macular degeneration (wet AMD), Wiskott Aldrich syndrome (WAS), X-linked myotubular myopathy, X-linked retinitis pigmentosa, and any combination thereof.

### Pharmaceutical Compositions and Methods of Administration

The present disclosure also provides pharmaceutical compositions comprising EVs (e.g., exosomes) of the present disclosure that are suitable for administration to a subject. The pharmaceutical compositions generally comprise a plurality of EVs (e.g., exosomes) comprising a biologically active molecule covalently linked to the plurality of EVs (e.g., exosomes) via a cleavable linker disclosed herein and a pharmaceutically-acceptable excipient or carrier in a form suitable for administration to a subject. Pharmaceutically acceptable excipients or carriers are determined in part by the particular composition being administered, as well as by the particular method used to administer the composition. Accordingly, there is a wide variety of suitable formulations of pharmaceutical compositions comprising a plurality of EVs (e.g., exosomes). See, e.g., Remington's Pharmaceutical Sciences, Mack Publishing Co., Easton, Pa. 18th ed. (1990). The pharmaceutical compositions are generally formulated sterile and in full compliance with all Good Manufacturing Practice (GMP) regulations of the U.S. Food and Drug Administration. In some aspects, the pharmaceutical composition comprises one or more chemical compounds, such as for example, small molecules covalently linked to an EV (e.g., exosome) of the present disclosure.

The present disclosure provides pharmaceutical compositions comprising an EV (e.g., exosome) of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

Acceptable carriers, excipients, or stabilizers are nontoxic to recipients (e.g., animals or humans) at the dosages and concentrations employed, and include buffers, such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight polypeptides (e.g., about 10 or less amino acid residues); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as a polysorbate (e.g., polysorbate 20, polysorbate 80), a poloxamer (e.g., block copolymers which consist of hydrophilic poly(ethylene oxide)(PEO) and hydrophobic poly(propylene oxide)(PPO), arranged in an A-B-A triblock structure to form PEO-PPO-PEO, such as those sold as PLURONIC™), or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated.

Typically, a pharmaceutical composition is formulated to be compatible with its intended route of administration. The EVs (e.g., exosomes) of the present disclosure can be administered by parenteral, topical, intravenous, oral, subcutaneous, intra-arterial, intradermal, transdermal, rectal, intracranial, intraperitoneal, intranasal, intratumoral, intrathecal, intraocular, intramuscular routes or as inhalants. In certain aspects, the pharmaceutical composition comprising EVs (e.g., exosomes) is administered intravenously, e.g., by injection.

In some aspects, the EVs (e.g., exosomes) are administered intravenously to the circulatory system of a subject. In some aspects, the EVs (e.g., exosomes) are infused in suitable liquid and administered into a vein of a subject. In some aspects, the EVs (e.g., exosomes) are administered intra-arterialy to the circulatory system of a subject. In some aspects, the EVs (e.g., exosomes) are infused in suitable liquid and administered into an artery of a subject. In some aspects, the EVs (e.g., exosomes) are administered to the subject by intrathecal administration. In some aspects, the EVs (e.g., exosomes) are administered via an injection into the spinal canal, or into the subarachnoid space so that it reaches the cerebrospinal fluid (CSF). In some aspects, the EVs (e.g., exosomes) are administered intratumorally into one or more tumors of a subject. In some aspects, the EVs (e.g., exosomes) are administered to a subject by intranasal administration. In some aspects, the EVs (e.g., exosomes) can be insufflated through the nose in a form of either topical administration or systemic administration. In certain aspects, the EVs (e.g., exosomes) are administered as nasal spray. In some aspects, the EVs (e.g., exosomes) are administered to the subject by intraperitoneal administration. In some aspects, the EVs (e.g., exosomes) are infused in suitable liquid and injected into the peritoneum of the subject. In some aspects, the intraperitoneal administration results in distribution of the EVs (e.g., exosomes) to the lymphatics. In some aspects, the intraperitoneal administration results in distribution of the EVs (e.g., exosomes) to the thymus, spleen, and/or bone marrow. In some aspects, the intraperitoneal administration results in distribution of the EVs (e.g., exosomes) to one or more lymph nodes. In some aspects, the intraperitoneal administration results in distribution of the EVs (e.g., exosomes) to one or more of the cervical lymph node, the inguinal lymph node, the mediastinal lymph node, or the sternal lymph node. In some aspects, the intraperitoneal administration results in distribution of the EVs (e.g., exosomes) to the pancreas. In some aspects, the EVs (e.g., exosomes) are administered to the subject by periocular administration. In some aspects, the EVs (e.g., exosomes) are injected into the periocular tissues. Periocular drug administration includes the routes of subconjunctival, anterior sub-Tenon's, posterior sub-Tenon's, and retrobulbar administration.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity, such as sodium chloride or dextrose. The pH can be adjusted with an acid or base, such as hydrochloric acid or sodium hydroxide. The preparation can be enclosed in ampoules, disposable syringes, or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, a mixture of polyoxyethylated triglycerides made by reacting ethylene oxide with castor oil (e.g., Cremophor EL™ (BASF, Parsippany, NJ)) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, or liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols, such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs (e.g., exosomes) of the present disclosure in an effective amount and in an appropriate solvent with one or a combination of ingredients enumerated herein, as desired. Generally, dispersions are prepared by incorporating the EVs (e.g., exosomes) into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs (e.g., exosomes) can be administered in the form of a depot injection or implant preparation which can be formulated in such a manner to permit a sustained or pulsatile release of the EVs (e.g., exosomes).

Systemic administration of compositions comprising EVs (e.g., exosomes) of the present disclosure can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs (e.g., exosomes) of the present disclosure is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105 (46): 17908 (2008)), or by intramuscular injection, by subcutaneous administration, by intratumoral injection, by direct injection into the thymus, or into the liver.

In certain aspects, the pharmaceutical composition comprising EVs (e.g., exosomes) of the present disclosure is administered as a liquid suspension. In certain aspects, the pharmaceutical composition is administered as a formulation that is capable of forming a depot following administration. In certain preferred aspects, the depot slowly releases the EVs (e.g., exosomes) into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

In some aspects, the pharmaceutically-acceptable carrier can comprise lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

In certain aspects, the preparation of EVs (e.g., exosomes) of the present disclosure is subjected to radiation, e.g., X rays, gamma rays, beta particles, alpha particles, neutrons, protons, elemental nuclei, UV rays in order to damage residual replication-competent nucleic acids. In certain aspects, the preparation of EVs (e.g., exosomes) of the present disclosure is subjected to gamma irradiation using an irradiation dose of more than 1, 5, 10, 15, 20, 25, 30, 35, 40, 50, 60, 70, 80, 90, 100, or more than 100 kGy. In certain aspects, the preparation of EVs (e.g., exosomes) of the present disclosure,, is subjected to X-ray irradiation using an irradiation dose (Gy) of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, or 10,000 Gy.

In some aspects, a pharmaceutical composition comprises one or more therapeutic agents and an EV (e.g., exosome) of the present disclosure, i.e., an EV comprising a cleavable linker disclosed herein. In certain aspects, the EVs (e.g., exosomes) are co-administered with of one or more additional therapeutic agents, in a pharmaceutically acceptable carrier. In some aspects, the pharmaceutical composition comprising the EV (e.g., exosome) is administered prior to administration of the additional therapeutic agents. In an aspect, the additional therapeutic agent can be a nucleic acid agent or a small molecule therapeutic agent, such as a hormonal therapeutic agent, a chemotherapeutic agent, an immunotherapeutic agent, an anti-inflammatory agent, or a medicament that inhibits the action of cell growth factors or cell growth factor receptors. In other aspects, the pharmaceutical composition comprising the EV (e.g., exosome) is administered after the administration of the additional therapeutic agents. In further aspects, the pharmaceutical composition comprising the EV (e.g., exosome) is administered concurrently with the additional therapeutic agents.

### Kits

The present disclosure also provides a kit comprising one or more EVs (e.g., exosomes) of the present disclosure and instructions for use. In some aspects, the kit, or product of manufacture contains a pharmaceutical composition described herein, which comprises at least one EV (e.g., exosome) of the present disclosure and instructions for use. In some aspects, the kit, or product of manufacture comprises at least one EV (e.g., exosome) of the present disclosure or a pharmaceutical composition comprising the EVs (e.g., exosomes) in one or more containers. One skilled in the art will readily recognize that the EVs (e.g., exosomes) of the present disclosure, pharmaceutical composition comprising the EVs (e.g., exosomes) of the present disclosure or combinations thereof can be readily incorporated into one of the established kit formats which are well known in the art.

In some aspects, the kit comprises EVs (e.g., exosomes) comprising one or more biologically active molecules, reagents to covalently attach the one or more biologically active molecules to the EVs (e.g., exosomes) via a cleavable linker, as disclosed herein, e.g., via conjugation or solid phase synthesis, and instructions to conduct the reaction to covalently attach the one or more biologically active molecules to the EVs (e.g., exosomes) via a cleavable linker disclosed herein.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press)(1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference ncludes the entire contents of the database entry in the most recent version of the database that was publicly available at the time the present application was filed. In case of conflict, the present specification, including definitions, will control. In addition, the materials, methods, and examples are illustrative only and not intended to be limiting.

### EXAMPLES

The following examples are provided for illustrative purposes only, and are not to be construed as limiting the scope or content of the invention in any way. The practice of the current invention will employ, unless otherwise indicated, conventional methods of protein chemistry, biochemistry, recombinant DNA techniques and pharmacology, within the skill of the art. Such techniques are explained fully in the literature. See, e.g., T.E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods in Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Example 1

This example describes the the loading of lipid-linker-ASO (-CPP), and the purification of exoASO.

The following materials were prepared.

Buffer CEF-01:15 mM Na2HPO4, 5 mM KH2PO+, 50 mM NaCl, 5% w/v sucrose, pH 7.2.

Buffer CEF-05:15 mM Na2HPO4, 5 mM KH2PO4, 150 mM NaCl, 5% w/v sucrose, pH 7.2.

Buffer CEF-04:15 mM Na2HPO+, 5 mM KH2PO4, 100 mM NaCl, 5% w/v sucrose, pH 7.2.

ULTRAFREE™-MC centrifugal filter units 0.22 um GV durapore (Millipore #UFC30GVOS, Burlington, MA) CAPTO™ Core 700 resin (50% v/v slurry)(Cytiva #17548102, Marlborough, MA).

Filter microplate, 96-well, polypropylene, with 0.45 μm polyvinylidene fluoride membrane (Agilent #200959-100, Santa Clara, CA).

Prepare Lipid-linker-ASO stock solution. Dissolve lipid-linker-ASO (-CPP) solid in CEF-04 buffer, aiming for a 500 μM final concentration. After vortex mixing, the solution was filtered by ULTRAFREE™-MC(Millipore, Burlington, MA) centrifugal filter units at a 2000xg centrifugation speed for 5 min. The actual stock solution concentration was determined by UA absorbance at A260.

Loading. An equal volume of the lipid-linker-ASO (-CPP) stock solution (350 μL) was fully mixed with exosomes (1.26E13 p/mL, 350 μL) and incubated at 37° C. for 24h. For each example, the ASO sequence was for mouse STAT6 (mSTAT6): 5′-TbsGbsAbsdGs (5MdC)-sdGsdAsdAsdTsdGsdGsdAs (5MdC) sdAsdGsdGsdTsCbsTbsTb-3′ (SEQ ID NO: 1124).

Purification. Captocore 700 resin (50% v/v slurry, 150 uL) was added into the wells of the filter microplate, followed by the addition of CEF-05 buffer (300 uL). The microplate was then centrifuged at 2000×g for 5 min to remove the storage solution of the resin and equilibrate theresin with CEF-05 buffer. This process was repeated at least 3 times to fully equilibrate the resin.

The reaction mixture was allowed to cool to room temperature for 30 min. Then it was added to the resin on the filter microplate and mixed on a plate shaker for at least 30 min, which gives sufficient contact time. After that, the microplate was then centrifuged at 2000×g for 5 min and the filtrate (purified exoASO) was collected. The filtrate was then filtered by ULTRAFREE™-MC (Millipore, Burlington, MA) centrifugal filter units at a 2000xg centrifugation speed for 5 min to ensure sterility.

The purified exoASO was characterized by the following parameters: (i) particle count/concentration/yield by nanoparticle tracking analysis (NTA); (ii) particle size and size distribution by dynamic light scattering (DLS); and (iii) loading density by RIBOGREEN™ assay (QUANT-IT™ RIBOGREEN™ RNA assay kit; Thermofisher, Waltham, MA).

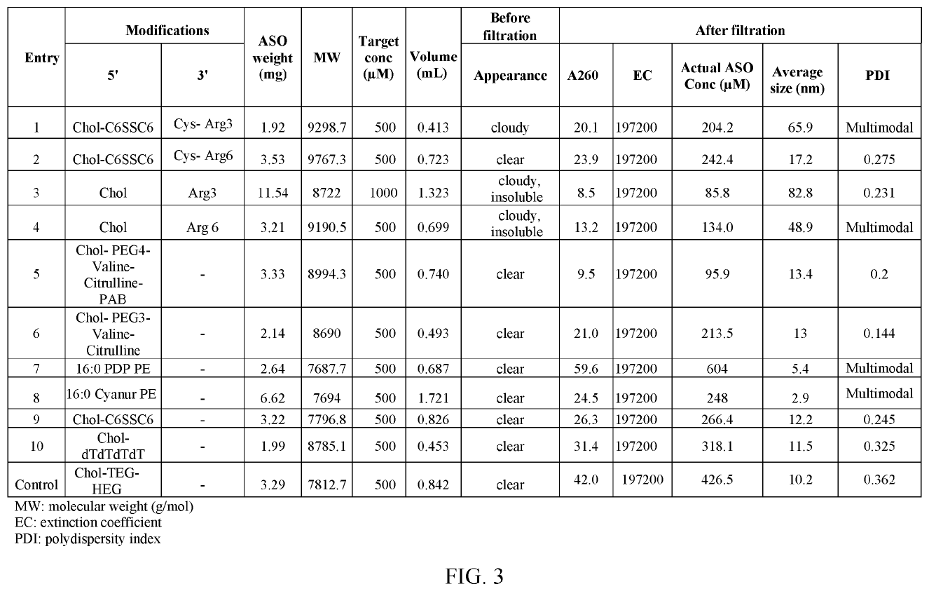

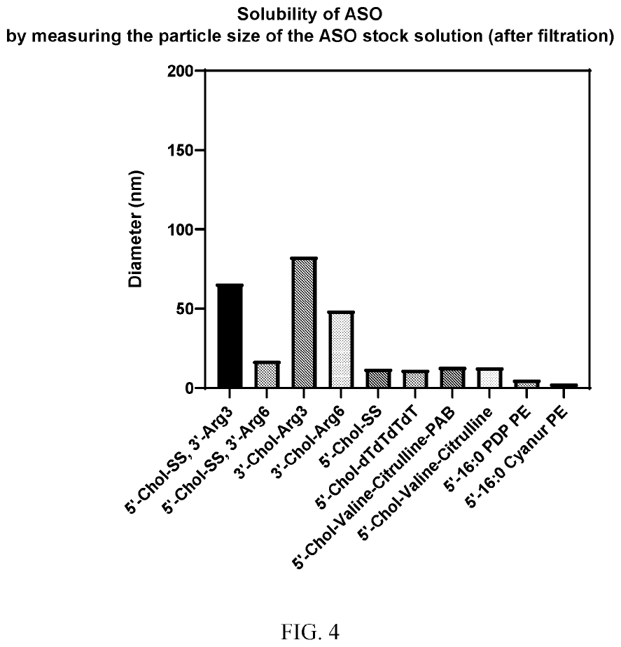

The average size and polydispersity index (PDI) of the lipid-linker-ASO stock solution are good indicators for aggregation. The lipid part is hydrophobic, and the ASO and CPP parts are hydrophilic. When conjugated together, the lipid-linker-ASO (-CPP) is likely to aggregate. The aggregation is evaluated by average diameter (nm) and PDI by dynamic light scattering (DLS). Lipid-linker-ASO stock solution with an average diameter <20 nm and PDI >0.25 would suggest decent solubility in the reconstitution buffer. Aggregation prevents lipid-linker-ASO from loading to exosomes and can interfere with the resin purification step. The characterization of the lipid-linker-ASO stock solutions are shown in FIG. 3. The solubtilities of exemplary ASOs as a function of the average diameter (nm) of ASO stock solutions are shown in FIG. 4.

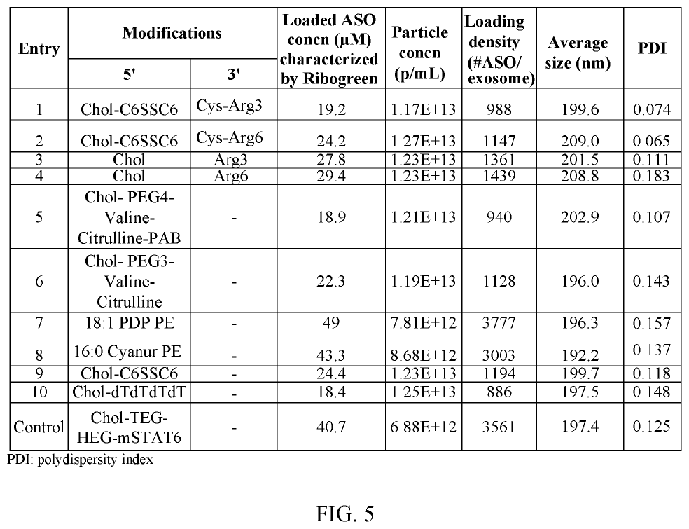

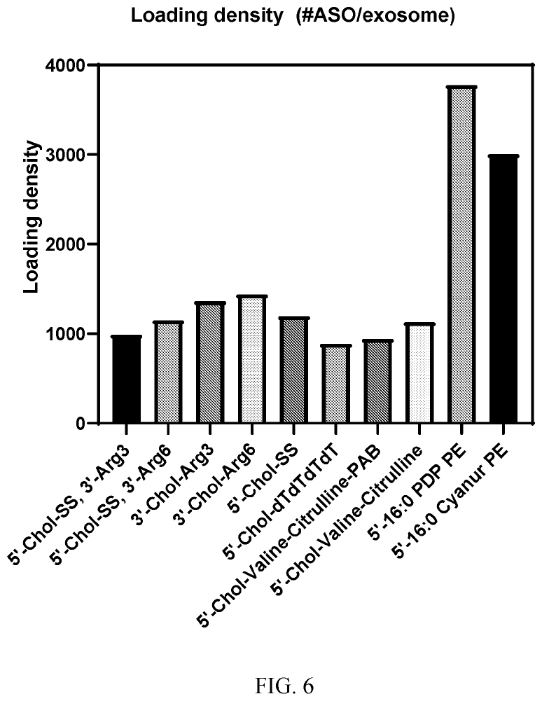

Loaded ASO concentration was characterized by RIBOGREEN™ assay (Thermofisher, Waltham, MA). Particle concentration was measured by NTA. Loading density was calculated by dividing the number of ASO by the number of exosome in a given concentration/volume. The average size and PDI were measured by DLS instrument. PDI<0.25 is the typical distribution for a rather uniform exosome population, meaning that there is no significant aggregation caused by the surface ASO loading. The results are shown in FIG. 5. The loading density of exemplary ASOs/exosome are shown in FIG. 6.

The loaded ASO concentration characterized by RIBOGREEN™ assay (Thermofisher, Waltham, MA) is useful for determining the dosing for in vitro and in vivo potency assay. The average particle size and the size distribution (PDI value) are important characteristics of exosomes. The surface of exosomes are modified with various ASO with cleavable linkers and cell-penetrating peptides, so it is important to monitor the particle size and distribution to make sure the payload will not have any negative impact on the physiochemical properties of exosomes.

The loading density of each individual lipid-linker-ASO (-CPP) varied from 886 to 3777, even though the constructures were all loaded to the exosome under the same condition. Different linkers and CPP structures affect the molecular interaction during loading. The hydrophobicity/hydrophilicity of the structures dictate their solubility. In general, if the lipid-linker-ASO (-CPP) showed good aqueous solubility, then it is more likely to result in a higher loading density as well as good filterability.

### Example 2

The exoASO potency/efficacy was examined by H1299 in vitro assay. H1299 is a lung cancer cell line containing the STAT6 target gene. The cells were allowed to propagate for several days. Once the cells reached the target density, the cells were treated with exoASO. The dosage of exoASO was calculated based on the loaded ASO concentration. After incubating the cells with exoASO for 48-72h at 37° C., the STAT6 gene knockdown was measured as an indicator of potency. H1299 was used as the primary in vitro assay system for high-throughput potency evaluation. Exemplary linkers from the H1299 assay were further evaluated in macrophage.

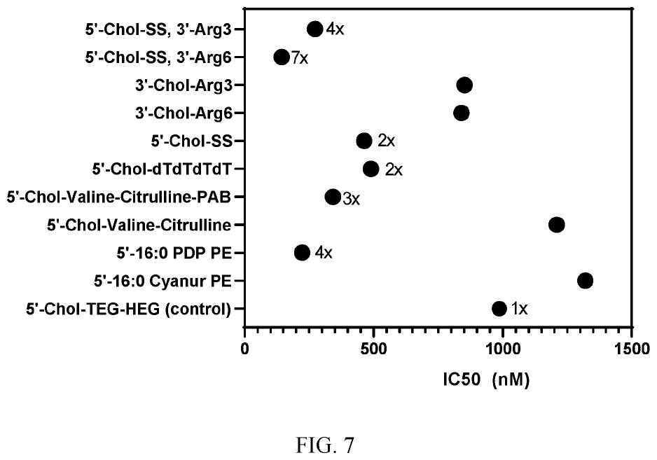

The IC₅₀ values of exemplary exoASOs modified with various cleavable linker and/or a cell penetrating peptide (CPP) compared to a control 5′-Chol-TEG-HEG with a phosphodiester linkage are set forth in FIG. 7.

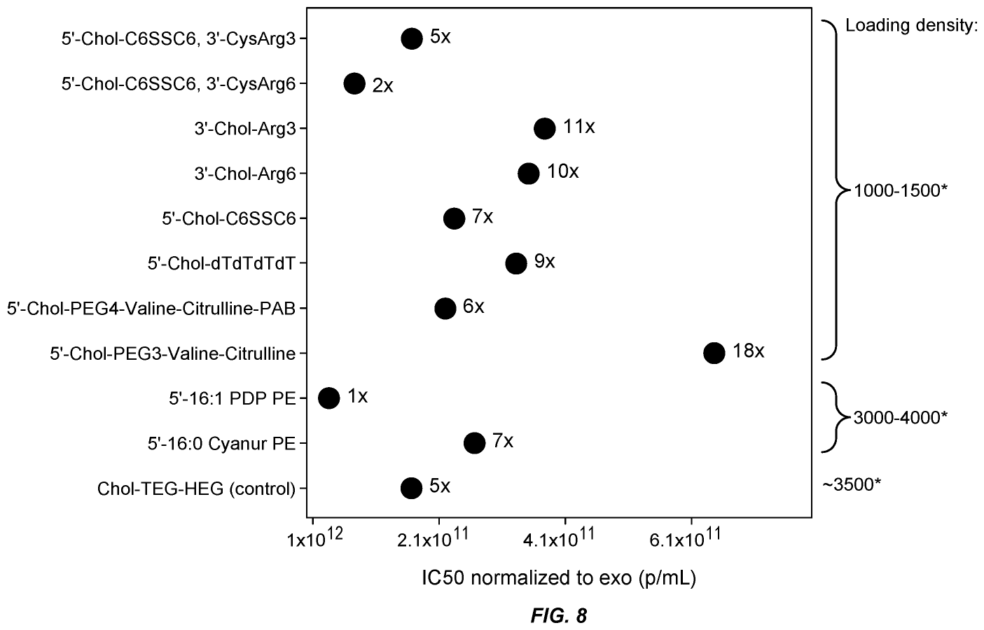

Cleavable linkers provide potency increase for exoASO. Each cleavable linker mechanism has a unique impact on the potency. As seen in FIG. 8, the degree of potency enhancement driven by the lipid and cleavable linker modifications follow this order: Phospholipid-SS >Chol-Val-Citrulline >Chol-SS=Chol-tetranucleotide.

The exoASO 5′-Chol-valine-citrullie-PAB (p-aminobenzyol) was compared to 5′-Chol-valine-citrulline. As seen in FIG. 7, despite both exoASOs including the cathepsin-cleavable linker Val-Cit, the linker Val-Cit-PAB, in which PAB is a self-immolative linker, is more potent that Val-Cit alone. A suggested mechanism of the dual cleavable linker is illustrated below.

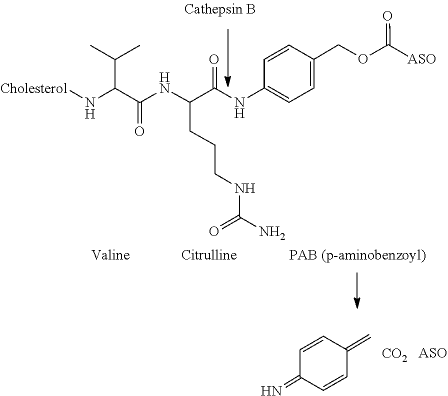

In a comparison of (5′-Chol-SS, 3′-Chol-Arg3 vs 3′-Chol-Arg3) and (5′-Chol-SS, 3′-Chol-Arg6 vs 3′-Chol-Arg6), it was observed that the addition of a disulfido (SS) bond or the combination of SS and a cell penentrating peptide (CPP) greatly improved potency. The exoASO potency increased in view of two unique mechanisms of CPP: enhanced uptake into the target cell and the endosomal escape capability. The endosomal escape brought by CPP is important for an exoASO to realize its full potency potential, because most of nanoparticle drug delivery systems suffer from the entrapment in the endo/lysosome.

In the exoASO 5′-16:0 PDP PE and 5′-16:0 Cyanur PE, the phospholipids (PE) with PDP or cyanur were used as the anchoring moiety rather than cholesterol. 16:0 PDP PE is a phospholipid with both phosphodiester and disulfido bonds. 16:0 Cyanur PE only contains a phosphodiester bond. As seen in FIG. 7, the exoASO with PDP PE had much higher potency that cyanur PE, suggesting that a disulfido bond is important in a phospholipid in improving the potency of exoASO.

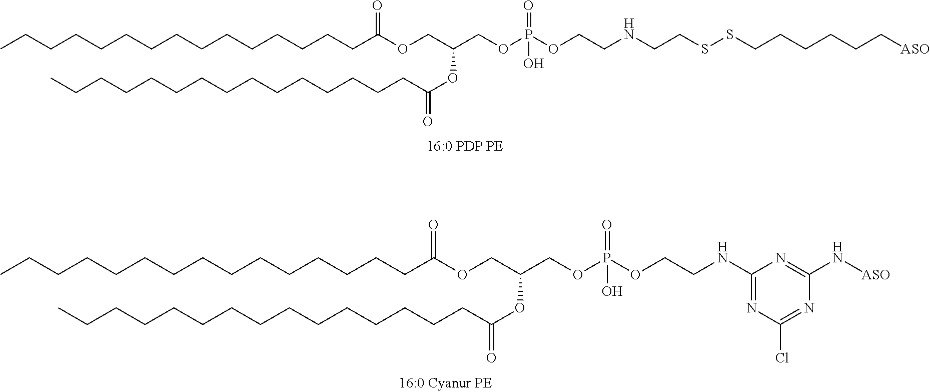

The IC₅₀ values normalized to loading density of the ASO per exosome are shown in FIG. 8. The overall efficacy of exoASO was determined by two factors: individual linker potency and loading density. The efficacy is a multiplication of both. Higher exoASO efficacy implies that a smaller dosage is needed to treat a subject with a particular disease or disorder in need of treatment. Lowering the effective dose can significantly reduce the doses need during manufacturing, thus lowering the overall costs of production and treatment.

### Example 3

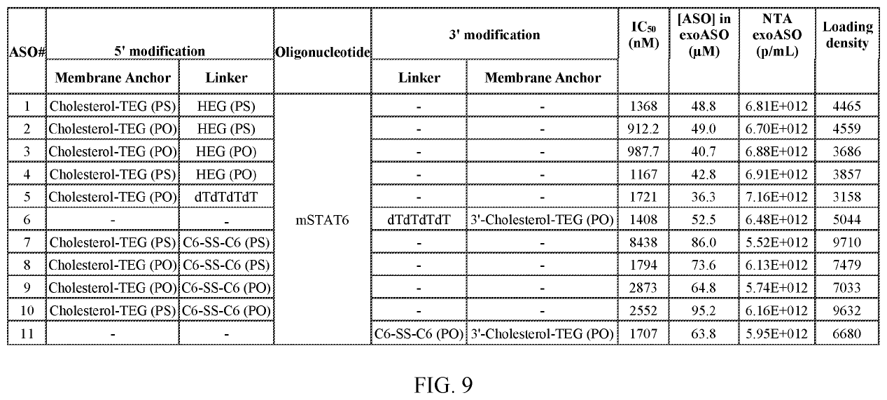

Following the procedure set forth in Example 2, the loading and characterization of exemplary exoASOs comprising various cleavable linkers are set forth in the table of FIG. 9.

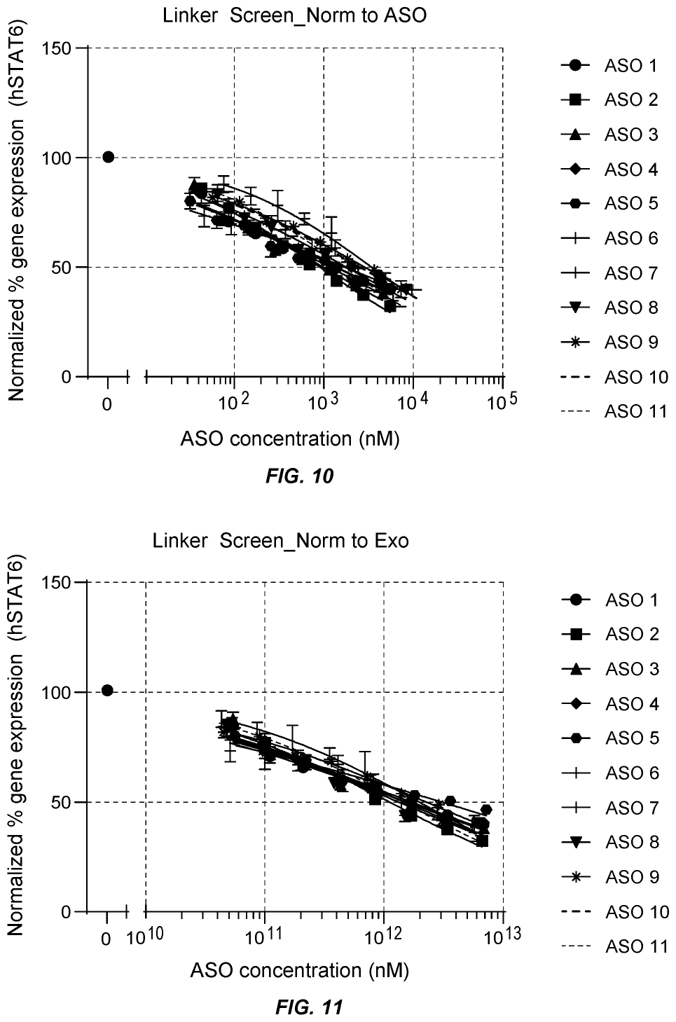

FIGS. 10 and 11 show the percent gene expression (hSTAT6) normalized to ASO concentration (nM) of various exoASOs for ASO numbers 1 to 11, as set forth in FIG. 9.

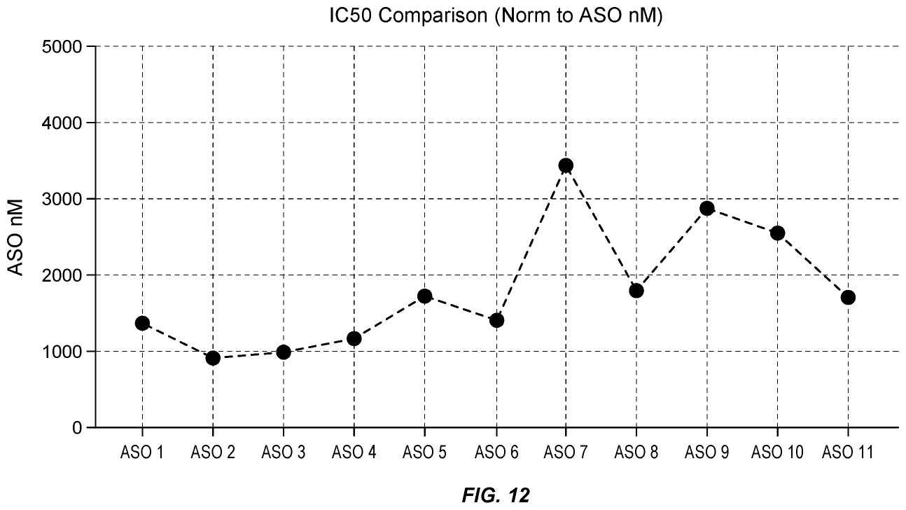

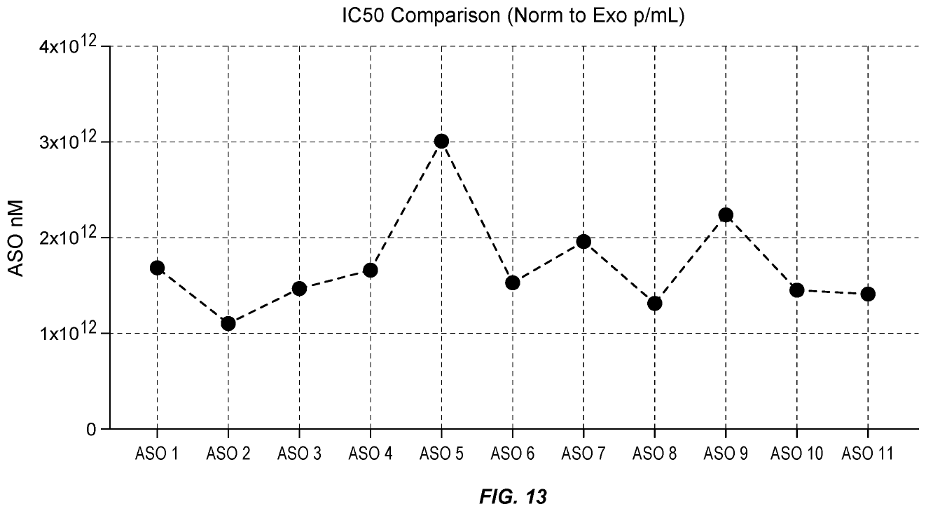

FIGS. 12 and 13 are graphs of IC₅₀ comparison normalized to ASO concentration (nM) for ASO numbers 1 to 11, as set forth in FIG. 9. FIG. 12 corresponds to the concentrations shown in FIG. 10, whereas FIG. 13 corresponds to the concentrations shown in FIG. 11.

For Chol-TEG-HEG (ASOs #1-4), there was no significant difference in potency by changing the locations of the phosphodiester (PO) or phosphorothioate (PS) linkage.

For Chol-tetranucleotide linkages (ASOs #5-6), there was no significant difference in potency when the cleavable linker was attached on the 5′ or 3′ end of the ASO.

For Chol-(PO) SS (PO)(ASOs #9 and #11), 3′ modification provided better potency than 5′ modification.

For Chol-SS (ASOs #7-10), an exoASO using one or two PO linkages exhibited better potency than the one with non-cleavable PS linkages (ASO #7). This suggests that, even a PO linkage is not as efficient as other cleavable linkers, a cleavable phosphodiester (PO) linkage is still slightly better than non-cleavable linker, such as phosphorothioate (PS). Based on these potency data and not wishing to be bound by any theory, it is believed that the release of ASO from the lipid anchor or exosome is important for ASO to be transported to its target and exert potency/function. On the other hand, if the ASO is tightly linked or trapped with its exosome carrier or endosome, then the ASO will not reach its full potency.

### Example 4

Based on the overall potency outcome obtained from both the H1299 and macrophage assays, exemplary linker selections were studied in an in vivo assay. ExoASOs consisting of one or two cleavable mechanisms, or with the combination of CPP, were evaluated in vivo. The STAT6 knockdown (KD) percentage was measured in a single dose 7-day treatment study. The dosage was calculated based on the loaded lipid-linker-ASO (-CPP) weight. Each exoASO was dosed at 5 μg ASO, except Chol-TEG-HEG was dosed at both 5 μg and 10 μg. After seven days, the mice were sacrificed, and the liver tissue was harvested to measure the STAT6 gene knockdown percentage. Each specific linker structure was measured with five mice and the resulting STAT6 KD percentage was taken as an average of the five.

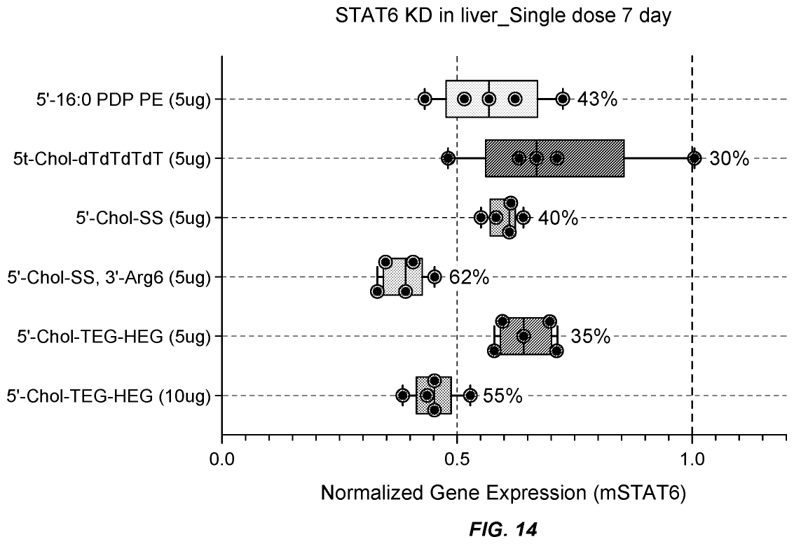

FIG. 14 shows the STAT6 knockdown percentage as an average of five measurements was labeled on the graph. A higher KD percentage is an indicator of higher potency or efficacy of the exoASO. ExoASOs with reducible linkers, e.g., 5′-Chol-SS and 5′-16:0 PDP PE, showed increased STAT6 KD compared to the Chol-TEG-HEG control at a 5 μg dose level. Among these exoASOs, the combination of disulfide and CPP showed the highest potency, roughly equal to 2× improvement compared to Chol-TEG-HEG. These data suggest that the incorporation of CPP likely improved the cellular uptake of exoASO and facilitated endosomal escape, which resulted in potency enhancement.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that can be cited throughout this application are hereby expressly incorporated by reference in their entirety for any purpose, as are the references cited therein.

What is claimed is:

1. An extracellular vesicle (EV) comprising a biologically active molecule (BAM) attached to the EV via an anchoring moiety (AM) according to Formula I or II: 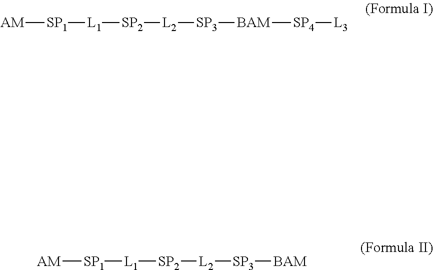
wherein
L₁, L₂, and L₃ are the same or different and each is an optional cleavable linkage; and
SP₁, SP₂, SP₃, and SP₄ are optional first, second, third, and fourth spacers, respectively, and
wherein at least one of L₁, L₂, and L₃ is present and comprises a cell penetrating peptide.

2. The EV of claim 1, wherein AM is covalently linked to BAM at the 5′ position.

3. The EV of claim 1, wherein AM is covalently linked to BAM at the 3′ position.

4. The EV of any one of claims 1-3, wherein the cell penetrating peptide comprises three or more argininyl moieties.

5. The EV of claim 4, wherein the cell penetrating peptide comprises three, six, or nine argininyl moieties.

6. The EV of claim 4 or 5, wherein the cell penetrating peptide further comprises at least one amino acid other than argininyl.

7. The EV of claim 6, wherein the at least one amino acid other than argininyl is cysteinyl, glycinyl, or a combination thereof.

8. The EV of any one of claims 1-3, wherein the cell penetrating peptide comprises a cyclic peptide, TAT, or Antp (antennapedia).

9. The EV of any one of claims 1-8, wherein L₁ is present and comprises the cell penetrating peptide.

10. The EV of any one of claims 1-8, wherein L₂ is present and comprises the cell penetrating peptide.

11. The EV of any one of claims 1-8, wherein L₃ is present and comprises the cell penetrating peptide.

12. The EV of any one of claims 1-11, wherein at least one cleavable linkage of L₁, L₂, and L₃ that does not comprise the cell penetrating peptide is present and is a cleavable linkage comprising a phosphodiester bond, a disulfido, a polypeptidyl, a polynucleotidyl, a pyrophosphato, or a silyl ether or a combination thereof.

13. The EV of claim 12, wherein the at least one cleavable linkage that does not comprise the cell penetrating peptide is a cleavable linkage comprising a phosphodiester.

14. The EV of claim 12, wherein the at least one cleavable linkage that does not comprise the cell penetrating peptide is a cleavable linkage comprising a disulfido.

15. The EV of claim 12, wherein the at least one cleavable linkage that does not comprise the cell penetrating peptide is a cleavable linkage comprising a polypeptidyl.

16. The EV of claim 15, wherein the polypeptidyl is selected from alanine-alanine-asparagine, valine-glycine, glycine-glycine, glutamic acid-valine-citrulline, aspartic acid-valine-citrulline, serine-valine-citrulline, lysine-valine-citrulline, glycine-glycine-glycine-valine-citrulline, cyclobutane-1,1-dicarboxamide-citrulline, and alanine-phenylalanine-lysine.

17. The EV of claim 12, wherein the at least one cleavable linkage that does not comprise the cell penetrating peptide is a cleavable linkage comprising a polynucleotidyl.

18. The EV of claim 17, wherein the polynucleotidyl is a trinucleotidyl or higher.

19. The EV of claim 18, wherein the polynucleotidyl is a tetranucleotidyl comprising dTdTdTdT, wherein dT is deoxythymidine.

20. The EV of claim 12, wherein the at least one cleavable linkage that does not comprise the cell penetrating peptide is a cleavable linkage comprising a pyrophosphato.

21. The EV of claim 12, wherein the at least one cleavable linkage that does not comprise the cell penetrating peptide is a cleavable linkage comprising a silyl ether.

22. The EV of claim 21, wherein the silyl ether comprises —OSiR¹R²O—, wherein R¹ and R² are the same or different and each is C₁₋₈ alkyl or aryl.

23. The EV of claim 22, wherein R¹ and R² are both isopropyl.

24. The EV of any one of claims 1-23, wherein AM comprises a sterol, a lipid, a vitamin, a peptide, or a combination thereof.

25. The EV of claim 24, wherein the sterol is cholesterol, thiocholesterol, ergosterol, 7-dehydrocholesterol, 24S-hydroxycholesterol, lanosterol, cycloartenol, fucosterol, saringosterol, campesterol, β-sitosterol, sitostanol, coprostanol, avenasterol, or stigmasterol.

26. The EV of claim 24 or 25, wherein the sterol is cholesterol.

27. The EV of claim 24, wherein the lipid is a fatty acid or a phospholipid.

28. The EV of claim 27, wherein the fatty acid is a straight chain fatty acid, a branched fatty acid, an unsaturated fatty acid, an unsaturated fatty acid, a hydroxyl fatty acid, a polycarboxylic acid, or any combination thereof.

29. The EV of claim 28, wherein the straight chain fatty acid is butyric acid, caproic acid, caprylic acid, capric acid, lauric acid, myristic acid, palmitic acid, or stearic acid.

30. The EV of claim 29, wherein the straight chain fatty acid is palmitic acid.

31. The EV of claim 24, wherein the phospholipid comprises 16:0 1,2-dipalmitoyl-sn-glycero-3-phosphoethanolamine-N-[3-(2-pyridyldithio) propionate] (16:0 PDP PE), 16:0 1,2-dipalmitoyl-sn-glycero-3-phosphoethanolamine-N-[4-(p-maleimidomethyl)cyclohexane-carboxamide] (16:0 PE MCC), or 16:0 1,2-dipalmitoyl-sn-glycero-3-phosphoethanolamine-N-(cyanur)(16:0 Cyanur PE).

32. The EV of claim 24, wherein the vitamin is tocopherol, tocotrienol, vitamin D, vitamin K, riboflavin, niacin, or pyridoxine.

33. The EV of claim 32, wherein the vitamin is tocopherol.

34. The EV of any one of claims 1-33, wherein AM is attached to an exterior surface of the EV.

35. The EV of any one of claims 1-34, wherein BAM comprises a peptidyl, a polypeptidyl, a polynucleotidyl, a protein, an antibody or an antigen binding fragment thereof, a residue of a chemical compound, or any combination thereof.

36. The EV of any one of claims 1-35, wherein BAM comprises an antisense oligonucleotidyl (ASO), siRNA, miRNA, shRNA, a nucleic acid, or any combination thereof.

37. The EV of claim 36, wherein BAM comprises an ASO.

38. The EV of claim 37, wherein the ASO targets a transcript.

39. The EV of claim 38, wherein the transcript is a STAT6 transcript, an EGFP transcript, a CEBP/β transcript, a STAT3 transcript, a KRAS transcript, an NRAS transcript, an NLPR3 transcript, or any combination thereof.

40. The EV of any one of claims 1-39, wherein SP₁, SP₂, SP₃, and SP₄ are the same or different and each comprises an alkylenyl, a polyoxyalkylenyl, a succinimido, a maleimido, an aryl, an ether, a carbonyl, a carboxylato, a carbamoyl, a thio, a thiocarbonyl, a thiocaramoyl, an amino, an amido, a hydrazido, a phosphorothioato, a 1,2,3-triazolyl, a dibenzoylcyclooctenyl, a bicyclononenyl, a p-aminobenzoyl, a p-aminobenzylcarbamato, or a combination thereof, and at least one of SP₁, SP₂, SP₃, and SP₄ is present.

41. The EV of claim 40, wherein at least one of SP₁, SP₂, SP₃, and SP₄ comprises C₁₋₈ alkylenyl, polyoxyalkenyl, a maleimido, a carbamoyl, a thio, an amido, a 1,2,3-triazolyl, a dibenzoylcyclooctenyl, a bicyclononenyl, a p-aminobenzoyl, a p-aminobenzylcarbamato, or a combination thereof.

42. The EV of claim 41, wherein at least one of SP₁, SP₂, SP₃, and SP₄ comprises C₁₋₆ alkylenyl.

43. The EV of claim 42, wherein at least one of SP₁, SP₂, SP₃, and SP₄ comprises a polyoxyalkylenyl that comprises 2 to 15 —OCH₂CH₂-repeat units.

44. The EV of claim 42 or 43, wherein at least one of SP₁, SP₂, SP₃, and SP₄ further comprises a carbamoyl, an amino, an amido, a thiosuccinimido, a 1,2,3-triazolylbicyclononenyl, or a combination thereof.

45. The EV of claim 39-44, wherein SP₁ is present.

46. The EV of any one of claims 39-45, wherein SP₂ is present.

47. The EV of any one of claims 39-46, wherein SP₃ is present.

48. The EV of any one of claims 39-47, wherein SP₄ is present.

49. The EV of claim 1, wherein Formula I or Formula II is 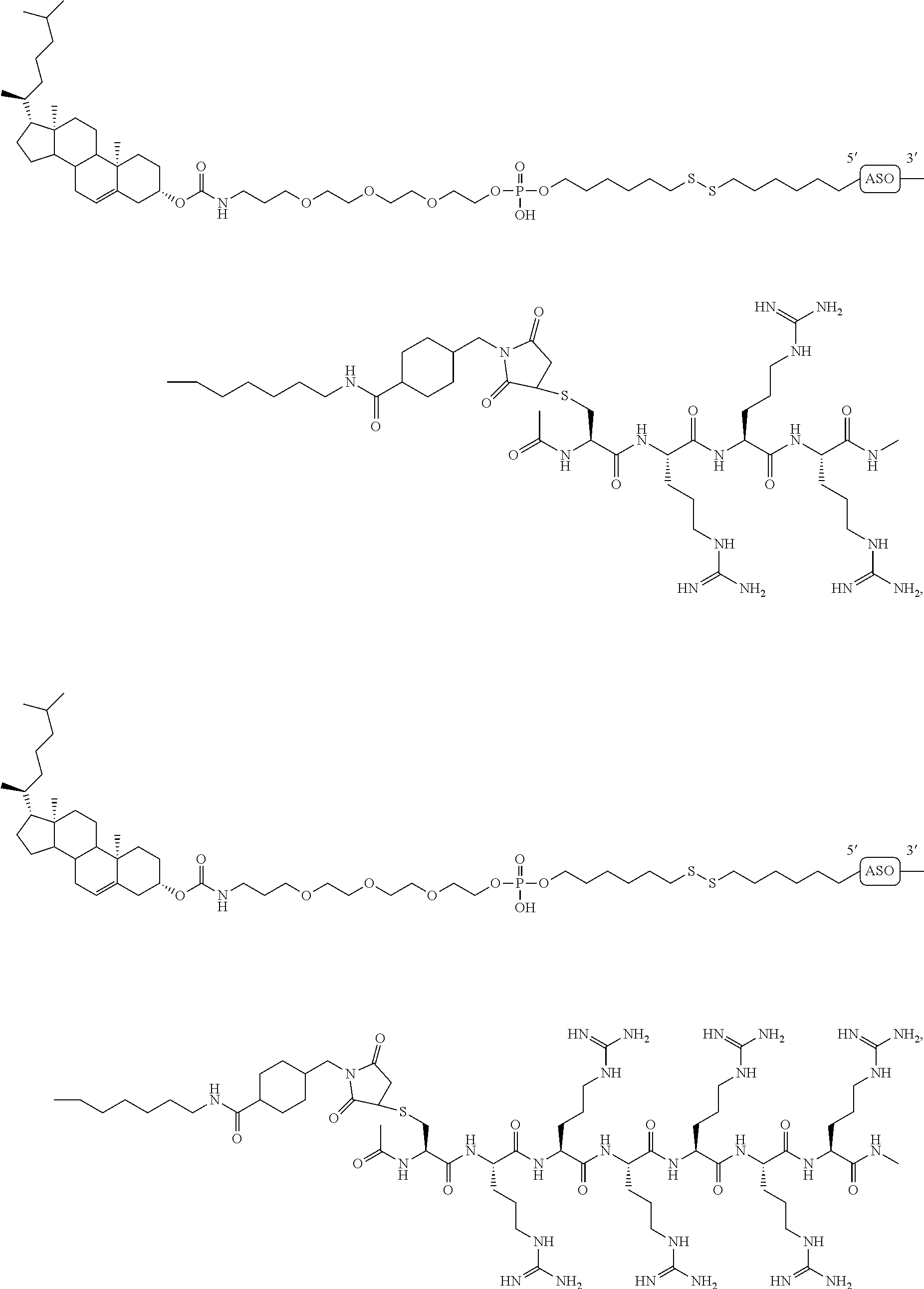 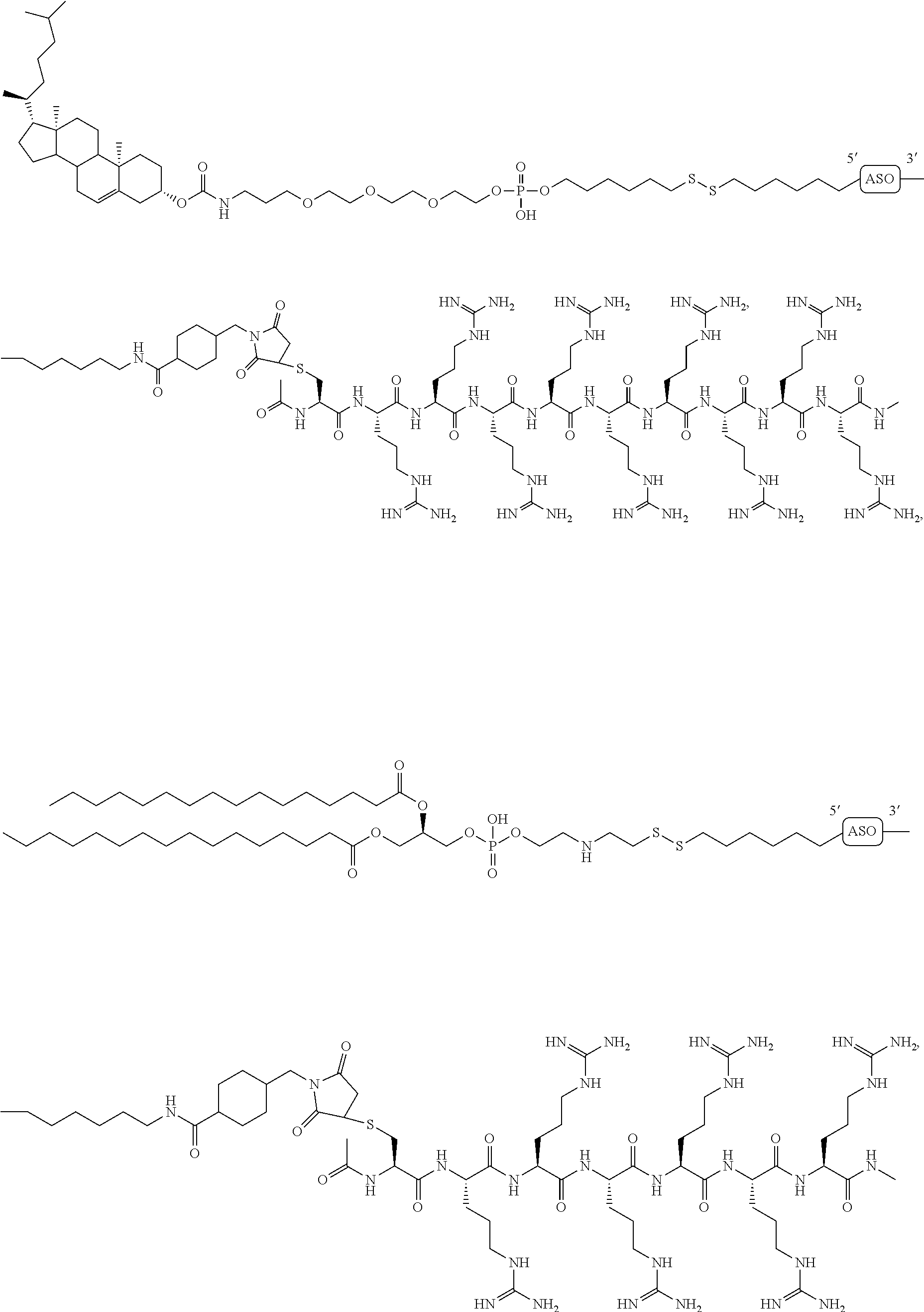 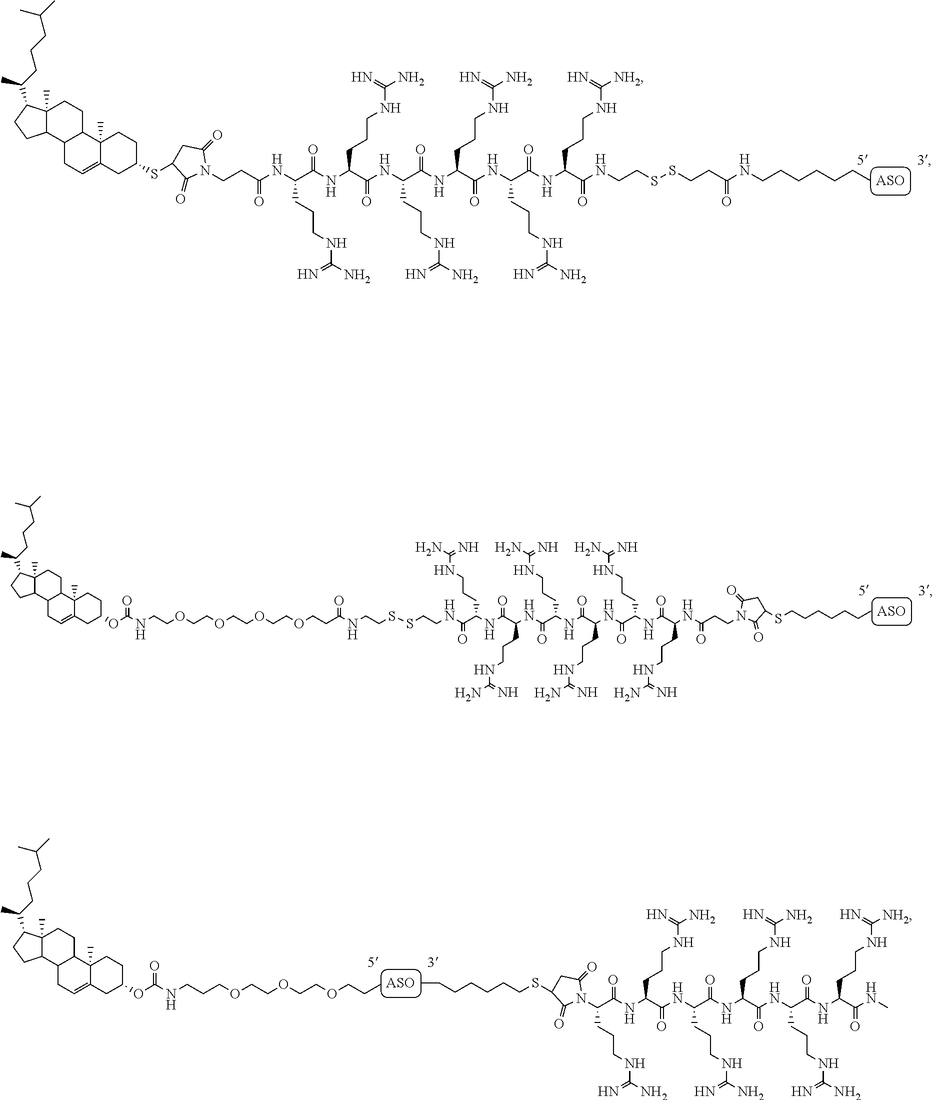 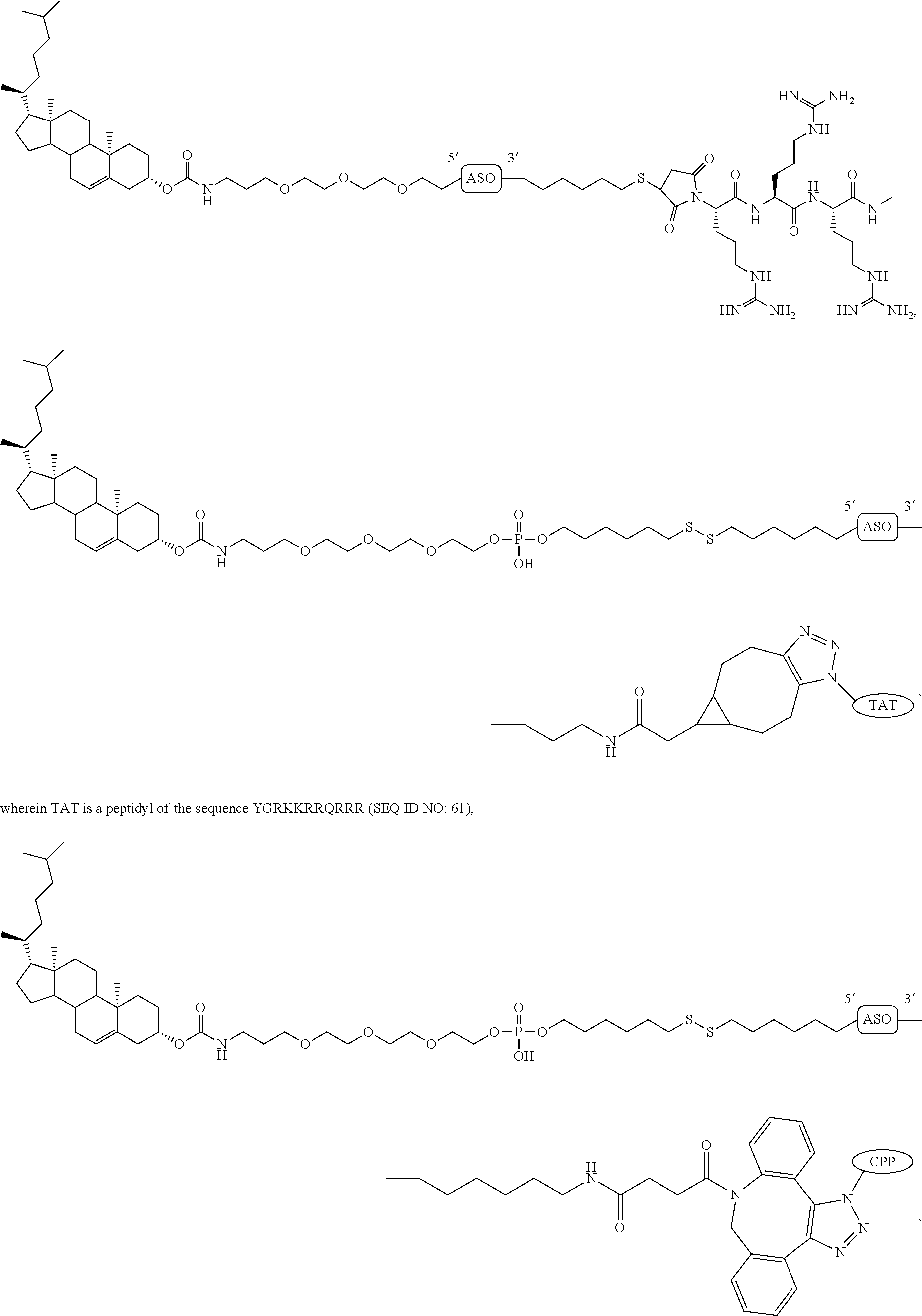
wherein the cell-penetrating peptide (CPP) is Antp (a peptidyl of the sequence RQIKIWFQNRRMKWKK (SEQ ID NO: 62)), R⁶ (a peptidyl of the sequence RRRRRR (SEQ ID NO: 87)), or cTAT (a peptide of sequence KRRRGRKKRRE (wherein K and E are connected to form a cyclic peptide)(SEQ ID NO: 88)), and

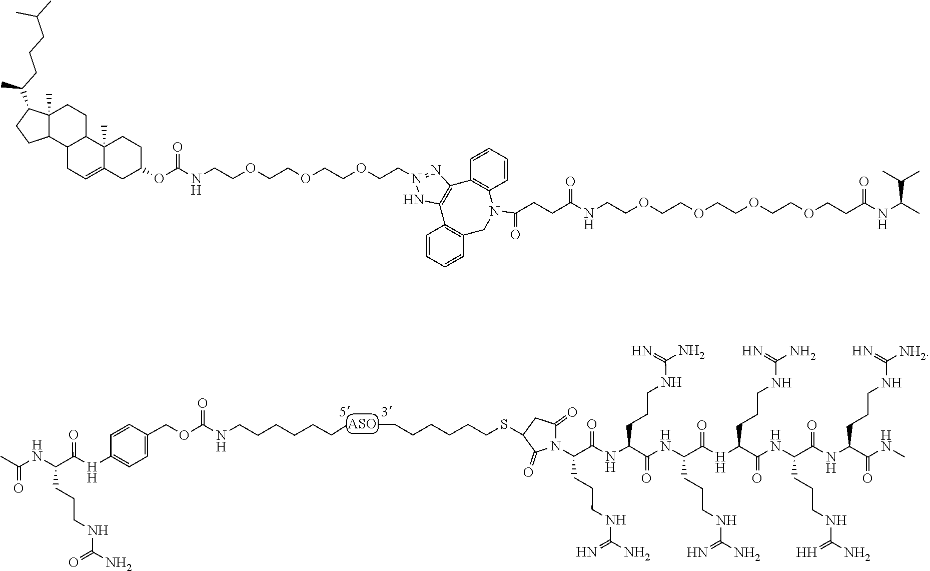

50. A pharmaceutical composition comprising the EV of any one of claims 1-49 and a pharmaceutically acceptable carrier.

51. A kit comprising the EV of any one of claims 1-49 or the pharmaceutical composition of claim 50 and instructions for use.

52. A method of treating or preventing a disease or disorder in a subject in need thereof comprising administering an effective amount of the EV of any one of claims 1-49 or the pharmaceutical composition of claim 50 to the subject.

53. The method of claim 52, wherein the disease or disorder is a cancer, graft-versus-host disease (GvHD), an autoimmune disease, an infectious disease, a fibrotic disease, an inflammatory disease, a neurodegenerative disorder, a central nervous disease, a muscular dystrophy disease, or a metabolic disease.
